(19) United States
(12) Patent Application Publication
(10) Pub. No.: US 2024/0023427 A1
(43) Pub. Date: Jan. 18, 2024

(54) ORGANIC COMPOUND AND ORGANIC LIGHT-EMITTING DEVICE

(71) Applicant: CANON KABUSHIKI KAISHA, Tokyo (JP)

(72) Inventors: NAOKI YAMADA, Tokyo (JP); HIROKI OHRUI, Tokyo (JP); JUN KAMATANI, Tokyo (JP); HIRONOBU IWAWAKI, Kanagawa (JP); YOSUKE NISHIDE, Kanagawa (JP); HIROKAZU MIYASHITA, Miyagi (JP)

(21) Appl. No.: 18/320,093

(22) Filed: May 18, 2023

(30) Foreign Application Priority Data
May 23, 2022 (JP) 2022-083821

(51) Int. Cl. H10K 85/60; C07C 15/30; C09K 11/06; C07D 209/86; C07D 333/76; C07D 307/91; C07D 409/04

(52) U.S. Cl. CPC H10K 85/626; C07C 15/30; C09K 11/06; C07D 209/86; H10K 85/6572; H10K 50/12; H10K 85/6576; C07D 307/91; H10K 85/6574; C07D 409/04; C07D 333/76

(57) ABSTRACT

An organic compound represented by the following formula (1):

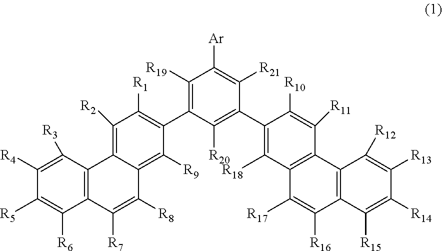

where in formula (1), Ar is a carbazolyl group, an aryl group, or a heterocyclic group containing a chalcogen element, provided that when Ar is a phenanthrenyl group, the phenanthrenyl group contains a substituent, and R₁ to R₂₁ are each independently selected from the group consisting of a hydrogen atom, a deuterium atom, a halogen atom, an alkyl group, an alkoxy group, a silyl group, an amino group, an aryl group, and a heterocyclic group.

### BACKGROUND OF THE INVENTION

### Field of the Invention

The present disclosure relates to an organic compound and an organic light-emitting device including the organic compound.

### Description of the Related Art

Organic light-emitting devices (hereinafter, also referred to as “organic electroluminescent devices” or “organic EL devices”) are electronic devices each including a pair of electrodes and an organic compound layer disposed between these electrodes. The injection of electrons and holes from these pairs of electrodes generates excitons in the light-emitting organic compound in the organic compound layer, and when the excitons return to the ground state, the organic light-emitting device emits light.

Recently, organic light-emitting devices have made remarkable progress and have achieved low-driving voltage, various emission wavelengths, and fast response time. The use thereof has enabled the development of thinner and lighter light-emitting apparatuses.

To date, many efforts have been made to create compounds suitable for organic light-emitting devices. This is because the creation of a compound having superior durability is important in providing a high-performance organic light-emitting device.

Compounds in which benzene rings are substituted with fused polycyclic groups have been created so far. Japanese Patent Laid-Open No. 2002-50481 (PTL 1) describes the following compound 1-A. Compound 1-A is a compound in which three phenanthrene molecules are attached to benzene at their 9-positions. U.S. Patent Application Publication No. 2015/0318487 (PTL 2) describes the following compound 1-B. Compound 1-B is a compound in which two triphenylene molecules are attached to benzene at their 2-positions and a phenanthrene molecule is attached to the benzene at its 3-position. PCT Japanese Translation Patent Publication No. 2019-512499 (PTL 3) describes the following compound 1-C. Compound 1-C is a compound in which two phenanthrene molecules are attached to a benzene ring at their 2-positions, and a triazine molecule having a phenyl group and a biphenyl group as substituents is attached to the benzene ring.

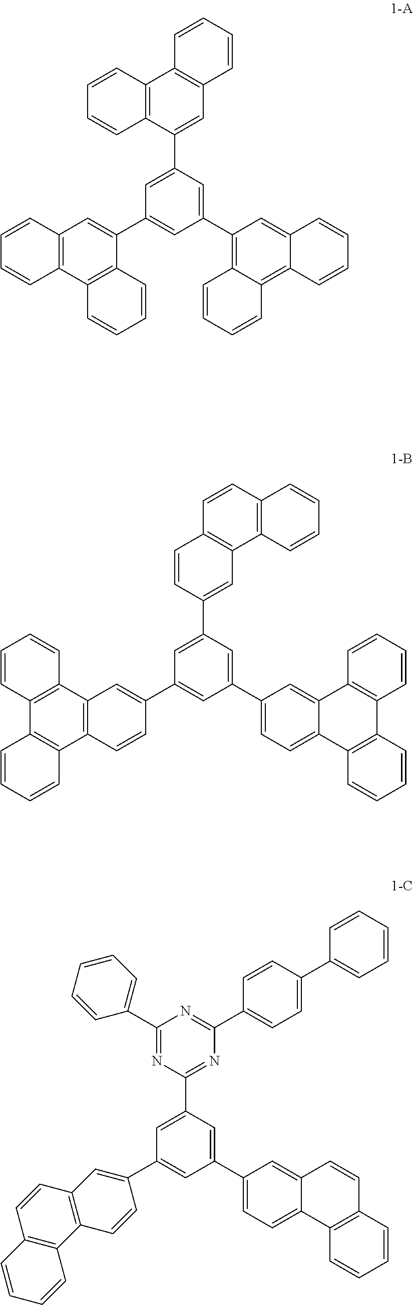

Compounds 1-A and 1-C have large ΔST. For this reason, when they are used in organic light-emitting devices, they are disadvantageous in terms of device lifetime. In this specification, ΔST indicates the energy difference between the lowest excited singlet energy (S1) and the lowest excited triplet energy (T1). Compound 1-B has low sublimability. For this reason, when compound 1-B is used in an organic light-emitting device, compound 1-B is disadvantageous in terms of device lifetime.

### SUMMARY OF THE INVENTION

The present disclosure has been made in light of the foregoing disadvantages and provides an organic light-emitting device excellent in device lifetime when an organic compound according to an embodiment of the present disclosure is used in the organic light-emitting device.

An organic compound according to an embodiment of the present disclosure is represented by the following formula (1).

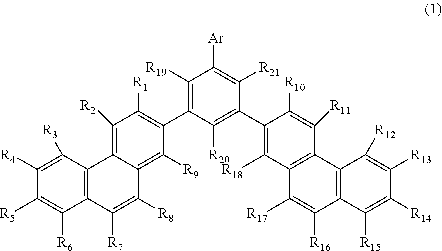

where in formula (1), Ar is a substituted or unsubstituted carbazolyl group, a substituted or unsubstituted aryl group, or a substituted or unsubstituted heterocyclic group containing a chalcogen element, provided that when Ar is a phenanthrenyl group, the phenanthrenyl group has a substituent, R₁ to R₂₁ are each independently selected from the group consisting of a hydrogen atom, a deuterium atom, a halogen atom, a substituted or unsubstituted alkyl group, a substituted or unsubstituted alkoxy group, a substituted or unsubstituted silyl group, a substituted or unsubstituted amino group, a substituted or unsubstituted aryl group, a substituted or unsubstituted heterocyclic group, and a substituted or unsubstituted carbazolyl group.

Further features of the present disclosure will become apparent from the following description of exemplary embodiments with reference to the attached drawings.

### BRIEF DESCRIPTION OF THE DRAWINGS

FIG. 1A is a schematic cross-sectional view of an example of a pixel of a display apparatus according to an embodiment of the present disclosure, and FIG. 1B is a schematic cross-sectional view of an example of a display apparatus including organic light-emitting devices according to an embodiment of the present disclosure.

FIG. 2 is a schematic view of an example of a display apparatus according to an embodiment of the present disclosure.

FIG. 3A is a schematic view of an example of an image pickup apparatus according to an embodiment of the present disclosure, and FIG. 3B is a schematic view of an example of an electronic apparatus according to an embodiment of the present disclosure.

FIG. 4A is a schematic view of an example of a display apparatus according to an embodiment of the present disclosure, and FIG. 4B is a schematic view of an example of a foldable display apparatus.

FIG. 5A is a schematic view of an example of a lighting apparatus according to an embodiment of the present disclosure, and FIG. 5B is a schematic view of an example of an automobile including an automotive lighting unit according to an embodiment of the present disclosure.

FIG. 6A is a schematic view illustrating an example of a wearable device according to an embodiment of the present disclosure, and FIG. 6B is a schematic view of an example of a wearable device according to an embodiment of the present disclosure, the wearable device including an image pickup apparatus.

FIG. 7A is a schematic view of an example of an image-forming apparatus according to an embodiment of the present disclosure, FIG. 7B is a schematic view of an example of an exposure light source of an image-forming apparatus according to an embodiment of the present disclosure, and FIG. 7C is a schematic view of an example of an exposure light source of an image-forming apparatus according to an embodiment of the present disclosure.

### DESCRIPTION OF THE EMBODIMENTS

In this specification, examples of halogen atoms include a fluorine atom, a chlorine atom, a bromine atom, and an iodine atom.

Examples of the chalcogen element include an oxygen atom, a sulfur atom, a selenium atom, and a tellurium atom.

An alkyl group may be an alkyl group having 1 to 20 carbon atoms. Non-limiting examples thereof include a methyl group, an ethyl group, a n-propyl group, an isopropyl group, a n-butyl group, a tert-butyl group, a sec-butyl group, an octyl group, a cyclohexyl group, a 1-adamantyl group, and a 2-adamantyl group.

An alkoxy group may be an alkoxy group having 1 to 10 carbon atoms. Non-limiting examples thereof include a methoxy group, an ethoxy group, a propoxy group, a 2-ethyloctyloxy group, and a benzyloxy group.

An aryl group may be an aryl group having 6 to 20 carbon atoms. Non-limiting examples thereof include a phenyl group, a naphthyl group, an indenyl group, a biphenyl group, a terphenyl group, a fluorenyl group, and a phenanthrenyl group.

A heterocyclic group may be a heterocyclic group having 3 to 20 carbon atoms. Non-limiting examples thereof include a furanyl group, a thiophenyl group, an oxazolyl group, a thiazolyl group, a carbazolyl group, a benzofuranyl group, a dibenzofuranyl group, a benzothiophenyl group, and a dibenzothiophenyl group.

An aryloxy group may be an aryloxy group having 6 to 20 carbon atoms.

Non-limiting examples thereof include a phenoxy group and a naphthoxy group.

A heteroaryloxy group may be a heteroaryloxy group having 3 to 20 carbon atoms. Non-limiting examples thereof include a thienyloxy group and a furanyloxy group.

Non-limiting examples of an amino group include an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, and an N-piperidyl group.

Non-limiting examples of a silyl group include a trimethylsilyl group and a triphenylsilyl group.

Non-limiting examples of substituents that may be further contained in the alkyl group, the alkoxy group, the aryl group, the heterocyclic group, the aryloxy group, the heteroaryloxy group, the amino group, and the silyl group include halogen atoms, such as a fluorine atom, a chlorine atom, a bromine atom, and an iodine atom; alkyl groups, such as a methyl group, an ethyl group, a n-propyl group, an isopropyl group, a n-butyl group, and a tert-butyl group; alkoxy groups, such as a methoxy group, an ethoxy group, and a propoxy group; amino groups, such as a dimethylamino group, a diethylamino group, a dibenzylamino group, a diphenylamino group, and a ditolylamino group; aryloxy groups, such as a phenoxy group; aromatic hydrocarbon groups, such as a phenyl group and biphenyl group; heterocyclic groups, such as a pyridyl group and pyrrolyl group; and a cyano group.

### (1) Organic Compound

An organic compound according to an embodiment of the present disclosure will be described.

The organic compound according to an embodiment of the present disclosure is an organic compound represented by formula (1).

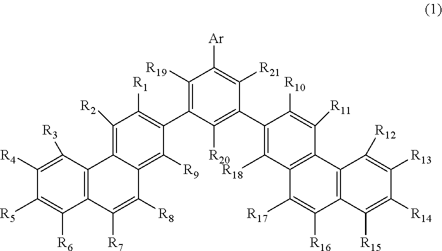

In formula (1), Ar is a substituted or unsubstituted aryl group, a substituted or unsubstituted heterocyclic group containing a chalcogen element, or a substituted or unsubstituted carbazolyl group, provided that when Ar is a phenanthrenyl group, the phenanthrenyl group contains a substituent. Ar may be a substituted or unsubstituted aryl group other than a phenanthrenyl group, a substituted or unsubstituted heterocyclic group containing a chalcogen element, or a substituted or unsubstituted carbazolyl group.

R₁ to R₂₁ is a hydrogen atom, a deuterium atom, a halogen atom, a substituted or unsubstituted alkyl group, a substituted or unsubstituted alkoxy group, a substituted or unsubstituted silyl group, a substituted or unsubstituted amino group, a substituted or unsubstituted aryl group, a substituted or unsubstituted heterocyclic group containing a chalcogen element, or a substituted or unsubstituted carbazolyl group.

The organic compound represented by formula (1) has the following feature.

(1-1) ΔST is small because the phenanthrene molecules are attached to the benzene ring at their 2-positions.

This feature will be described below.

(1-1) ΔST is small because the phenanthrene molecules are attached to the benzene ring at their 2-positions.

In inventing the organic compound according to an embodiment of the present disclosure, the inventors have found that ΔST is reduced by attaching phenanthrene to benzene at the 2-position.

Table 1 presents the calculated values of S1 and T1 of compound 1, which is an organic compound according to an embodiment of the present disclosure, and comparative compounds 1 and 2, and values of ΔST calculated therefrom. Compound 1, which is a compound according to an embodiment of the present disclosure, is a compound in which two phenanthrene molecules are attached to a benzene ring at their 2-positions. Comparative compound 1 is a compound in which two phenanthrene molecules are attached to a benzene ring at their 9-positions. Comparative compound 2 is a compound in which two phenanthrene molecules are attached to a benzene ring at their 3-positions.

The above calculation was performed using molecular orbital calculation. As the molecular orbital calculation method, the density functional theory (DFT), which is widely used at present, was used with the B3LYP functional and 6-31G* as the basis function. The molecular orbital calculation method was performed using Gaussian 09 (Gaussian 09, Revision C.01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc., Wallingford CT, 2010), which is widely used at present.

TABLE 1

| | Structure | S1 (eV) | ΔST (eV) | T1 (eV) |
|---|---|---|---|---|
| Compound 1 | 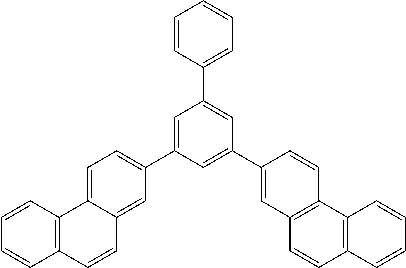 [SMILES: c1ccc(-c2cc(-c3ccc4c(ccc5ccccc54)c3)cc(-c3ccc4c(ccc5ccccc54)c3)c2)cc1] | 3.83 | 1.16 | 2.66 |
| Comparative compound 1 | 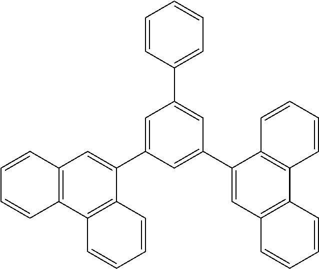 [SMILES: c1ccc(-c2cc(-c3cc4ccccc4c4ccccc34)cc(-c3cc4ccccc4c4ccccc34)c2)cc1] | 3.93 | 1.27 | 2.66 |
| Comparative compound 2 | 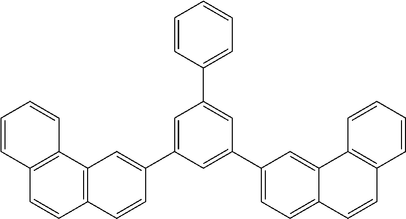 [SMILES: c1ccc(-c2cc(-c3ccc4ccc5ccccc5c4c3)cc(-c3ccc4ccc5ccccc5c4c3)c2)cc1] | 3.88 | 1.27 | 2.60 |

Table 1 indicates that S1 of compound 1 was 3.83 eV. S1 of comparative compound 1 was 3.93 eV, and S1 of comparative compound 2 was 3.88 eV. It can be seen that S1 of compound 1 is the lowest value. As compared with the case where the phenanthrene molecules are attached to the benzene ring at their 3- or 9-positions, when the phenanthrene molecules are attached to the benzene ring at their 2-positions, the S1 is low because the conjugation is long.

T1 of compound 1 was 2.66 eV. T1 of comparative compound 1 was 2.66 eV, and T1 of comparative compound 2 was 2.60 eV. The value of T1 of compound 1 was comparable to those of comparative compounds 1 and 2.

Thus, the organic compound according to an embodiment of the present disclosure has low S1 and comparable T1, compared with comparative compounds 1 and 2. That is, the organic compound according to an embodiment of the present disclosure is an organic compound having smaller ΔST than comparative compounds 1 and 2.

Here, the effect of small ΔST will be described.

A phosphorescent device is an organic light-emitting device that uses T1 energy for light emission. A host material used in a light-emitting layer of the organic light-emitting device can have higher T1 energy than a light-emitting material that emits phosphorescent light.

An organic compound with higher T1 energy tends to have higher S1 energy.

Higher S1 energy indicates a larger band gap. In this specification, the band gap refers to an energy difference between the highest occupied molecular orbital (HOMO) and the lowest unoccupied molecular orbital (LUMO). A large band gap of a host material in the light-emitting layer leads to a deterioration in hole and electron injection properties from the peripheral layers to the light-emitting layer. This results in a higher device voltage and a shorter device lifetime due to unnecessary charge accumulation. A small band gap of a host material in the light-emitting layer leads to an improvement in hole and electron injection properties from the peripheral layers to the light-emitting layer. This makes it possible to reduce the device voltage and inhibit a reduction in device lifetime due to unnecessary charge accumulation.

Thus, an organic compound having high T1 energy and low S1 energy, i.e., small ΔST, can be used as a host material for the light-emitting layer of the organic light-emitting device.

Ar in the organic compound represented by formula (1) can contain no electron-withdrawing substituent. Examples of the electron-withdrawing substituent include a pyridyl group, a pyrimidinyl group, a pyrazinyl group, and a triazinyl group. When Ar contains such a substituent, the HOMO level is lowered (farther from the vacuum level), thus deteriorating the hole injection properties from a hole transport layer side. For this reason, the use of such Ar in an organic light-emitting device results in a higher drive voltage and lower durability. In contrast, when the organic compound according to an embodiment of the present disclosure does not contain any of these substituents, the HOMO level thereof is higher (closer to the vacuum level) than that of the organic compound containing such an electron-withdrawing substituent. Thus, the hole injection barrier into the light-emitting layer is reduced, and it is possible to inhibit an increase in the device voltage and a deterioration in the device durability due to unnecessary charge accumulation.

As described above, when the organic compound according to an embodiment of the present disclosure is used as a host material of a light-emitting layer of an organic light-emitting device, it is possible to provide an organic light-emitting device that can be driven at a low voltage and are excellent in luminous efficiency and device lifetime.

Specifically, Ar in formula (1) can have a structure represented by the following formula (2).

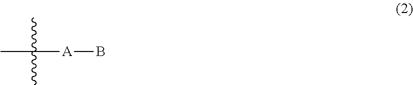

In formula (2), A is a substituted or unsubstituted phenylene group, a substituted or unsubstituted biphenylene group, or a direct bond.

Formula (2) can be represented by any one of the following formulae (3) to (9). In formulae (3) to (9), each X is a chalcogen element. R₃₀ to R₁₀₈ are each independently selected from the group consisting of a hydrogen atom, a deuterium atom, a halogen atom, a substituted or unsubstituted alkyl group, a substituted or unsubstituted alkoxy group, a substituted or unsubstituted silyl group, a substituted or unsubstituted amino group, a substituted or unsubstituted aryl group, and a substituted or unsubstituted heterocyclic group.

In formula (9), when A is a direct bond, at least one of R₁₀₀ to R₁₀₈ contains a substituent. When at least one of R₁₀₀ to R₁₀₈ contains the substituent, the symmetry of the molecule is reduced. This can result in low crystallinity of the molecules low sublimation temperature. In addition, molecular association can be inhibited; thus, the crystallization of the molecule can be inhibited. The substituent contained in at least one of R₁₀₀ to R₁₀₈ can be a group other than electron-withdrawing groups. The substituent can be an alkyl group having 1 to 10 carbon atoms, can be an alkyl group having 1 to 4 carbon atoms, or can be a methyl group or a tert-butyl group.

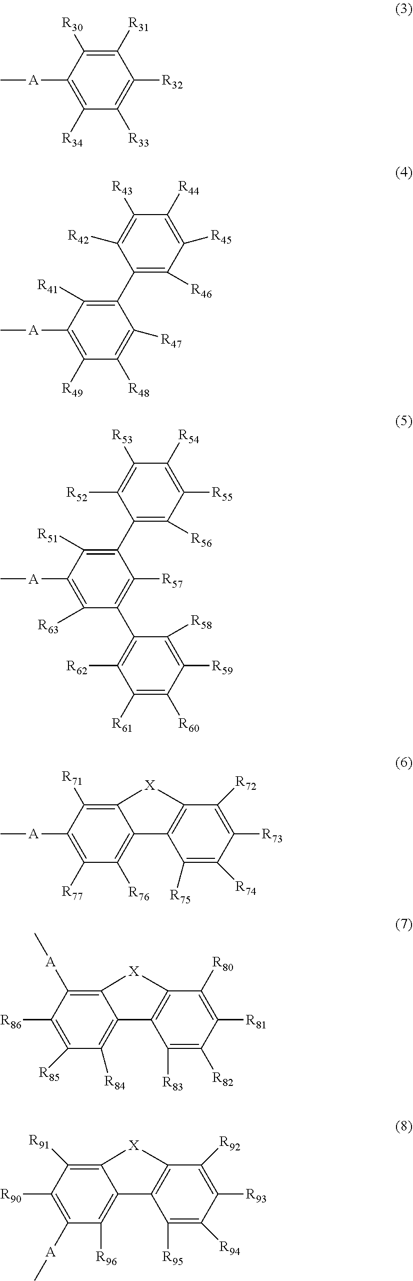

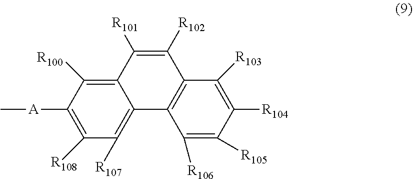

A substituent further contained in Ar can be a deuterium atom, a carbazole skeleton, an alkyl group having 1 to 4 carbon atoms, an aryl group having 6 to 18 carbon atoms, a chalcogen element-containing heterocyclic group having 8 to 12 carbon atoms, a silyl group containing an aryl group, or an amino group containing an alkyl group or an aryl group, can be a carbazole skeleton, an alkyl group having 1 to 4 carbon atoms, an aryl group having 6 to 10 carbon atoms, or a heterocyclic group having 6 to 12 carbon atoms, or can be a methyl group, a tert-butyl group, or a carbazolyl group.

Moreover, the organic compound according to an embodiment of the present disclosure can have the following features.

(1-2) The organic compound according to an embodiment of the present disclosure contains no SP3 carbon and thus has excellent stability against holes and electrons.
(1-3) Sublimability is excellent because at least one of R₁ to Rig is a substituent other than a hydrogen atom.
(1-4) The organic compound according to an embodiment of the present disclosure contains a heteroatom and thus has excellent charge transport properties.
(1-5) Sublimability is excellent because Ar is an aryl group other than a phenanthrene skeleton.

These features are described below.

(1-2) The organic compound according to an embodiment of the present disclosure contains no SP3 carbon and thus has excellent stability against holes and electrons.

When the organic compound according to an embodiment of the present disclosure contains no SP3 carbon, the organic compound has excellent stability against holes and electrons. Carbon-hydrogen bonds and carbon-carbon bonds with SP3 carbons have relatively low bond energies. Thus, cation radicals and anion radicals are easily generated by repeated exposure to holes and electrons due to energization. These radical species serve as active species for excitons, causing quenching. The structure containing no SP3 carbon has excellent bond stability and is less likely to generate radical species. Therefore, the organic compound is stable against holes and electrons.

(1-3) Sublimability is excellent because at least one of R₁ to R₁₉ is a substituent other than a hydrogen atom.

In formula (1), when at least one of R₁ to R₁₉ is a substituent other than a hydrogen atom, the symmetry of the molecule is reduced. This results in low crystallinity of the molecules and low sublimation temperature. In addition, the overlapping of the fused rings between molecules can be impeded, thereby inhibiting the crystallization of the molecules. Thus, in the organic compound according to an embodiment of the present disclosure, at least one of R₁ to R₁₉ can be a substituent other than a hydrogen atom. When a substituent other than a hydrogen atom is contained, the phenanthrene skeleton having high planarity can contain a substituent. Specifically, at least one selected from the group consisting of R₄, R₅, R₆, R₇, R₁₃, R₁₄, and Rib can be a substituent. At least one selected from the group consisting of R₄, R₅, R₁₃, and R₁₄ can be a substituent. The substituent can have a bulkier structure than a hydrogen atom. Specifically, the substituent can be a deuterium atom, an alkyl group having 1 to 4 carbon atoms, or an aryl group having 6 to 12 carbon atoms, or can be a methyl group, a tert-butyl group, or a phenyl group. In particular, when the substituent is an alkyl group, the organic compound according to an embodiment of the present disclosure can further impede the overlapping of the fused rings between the molecules and thus can have excellent sublimability. Moreover, the alkyl group is an electron-donating substituent. This can result in a lower HOMO level (closer to the vacuum level). Thus, excellent hole injection properties can be obtained, and the device voltage can be lowered.

(1-4) The organic compound according to an embodiment of the present disclosure contains a heteroatom and thus has excellent charge transport properties.

The organic compound according to an embodiment of the present disclosure contains a heteroatom and thus has excellent charge transport properties. The hetero atom can be an oxygen atom, a sulfur atom, or a nitrogen atom. The lone pair possessed by such an atom can improve charge transport properties. Thus, there is an advantage that it is easy to adjust the carrier balance. The heteroatom can be contained in a fused ring.

A heteroatom-containing fused ring can be a benzofuran skeleton, a dibenzofuran skeleton, a benzothiophene skeleton, a dibenzothiophene skeleton, or a carbazole skeleton, can be a dibenzofuranyl group, a dibenzothiophene skeleton, or a carbazole skeleton, or can be a carbazolyl group. From the viewpoint of achieving good bond stability, the dibenzofuran skeleton and the dibenzothiophene skeleton can be attached at the 2-, 3- or 4-position.

(1-5) Sublimability is excellent because Ar is an aryl group other than a phenanthrene skeleton.

The organic compound according to an embodiment of the present disclosure has excellent sublimability because Ar is an aryl group other than a phenanthrene skeleton. If Ar is a phenanthrene skeleton, the symmetry of the molecule is high. This results in high crystallinity of the molecules and high sublimation temperature. In contrast, when Ar is an aryl group other than phenanthrene, the symmetry of the molecule is lowered. This results in low crystallinity of the molecules and low sublimation temperature. Thus, Ar can be an aryl group or a heterocyclic group other than the phenanthrene skeleton, and can be represented by any one of formulae (3) to (8).

While specific examples of the organic compound according to an embodiment of the present disclosure are illustrated below, the present invention is not limited thereto.

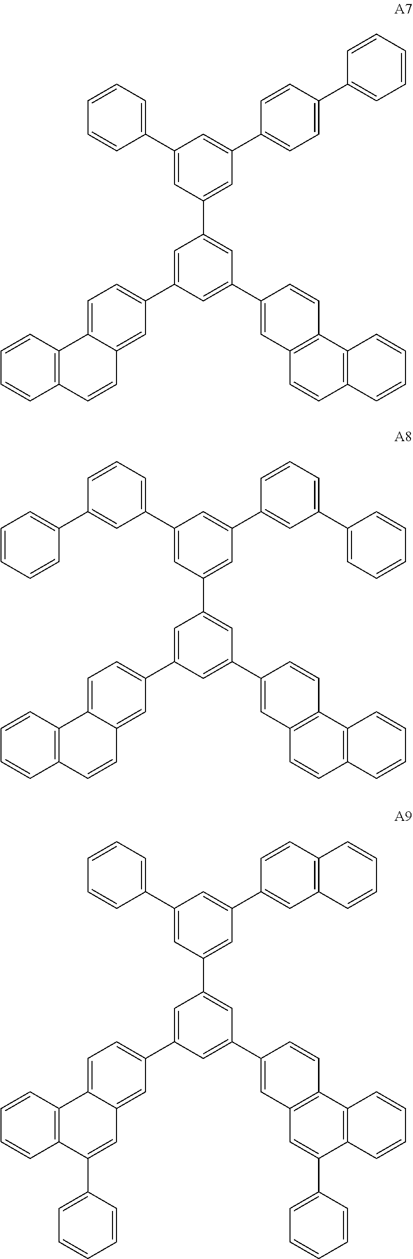

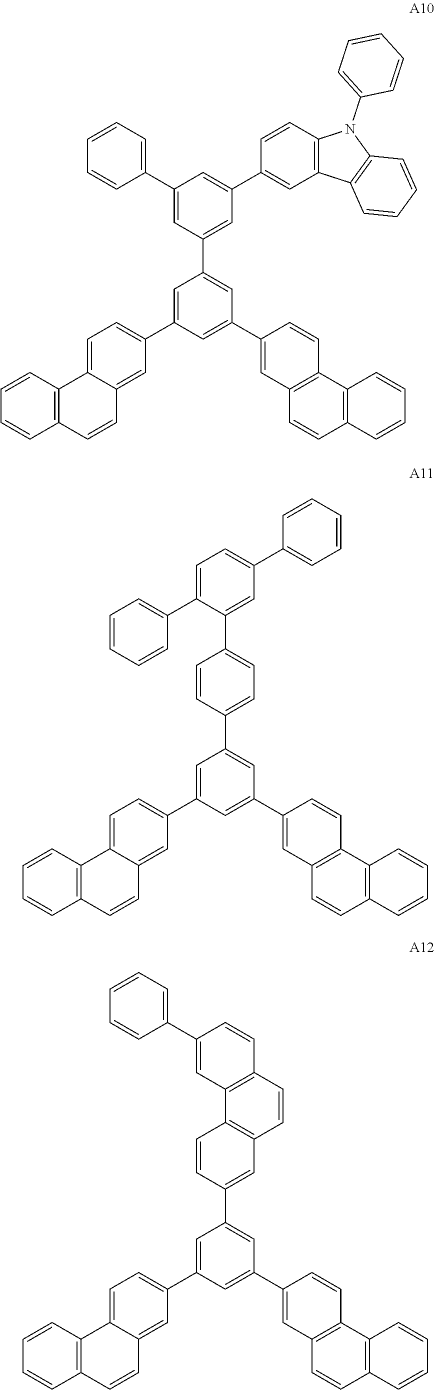

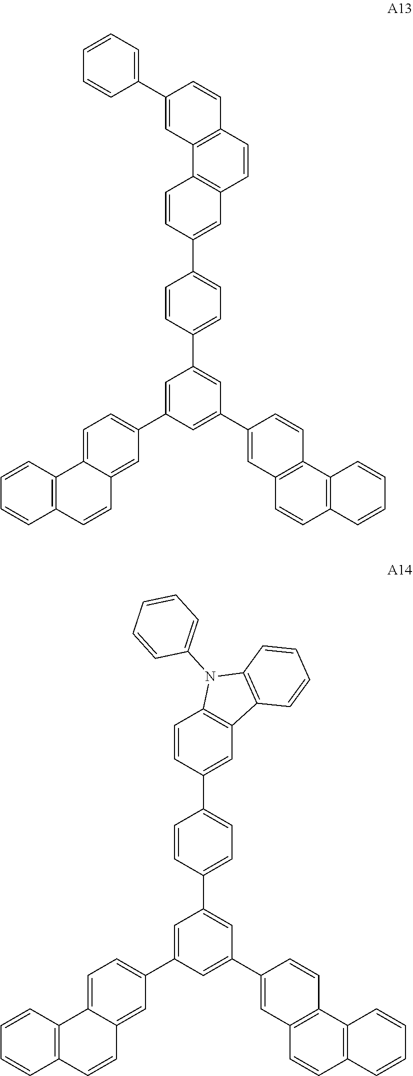

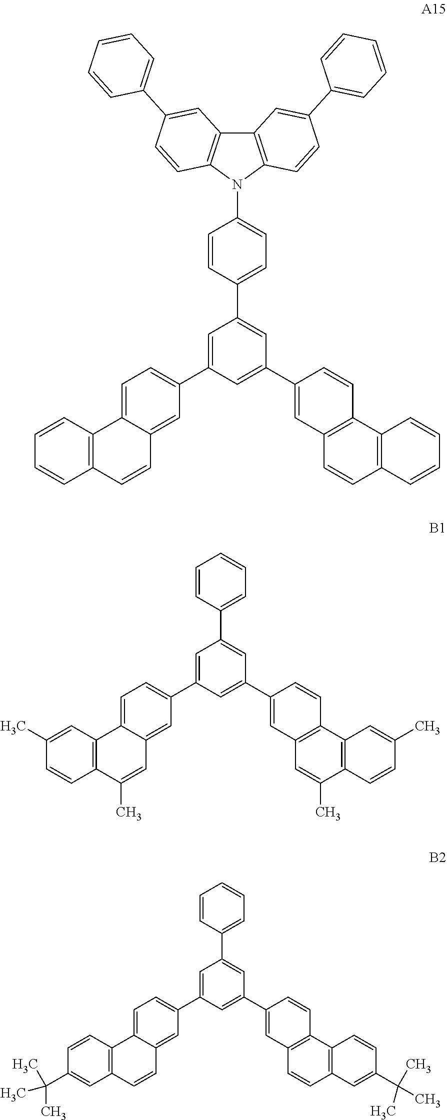

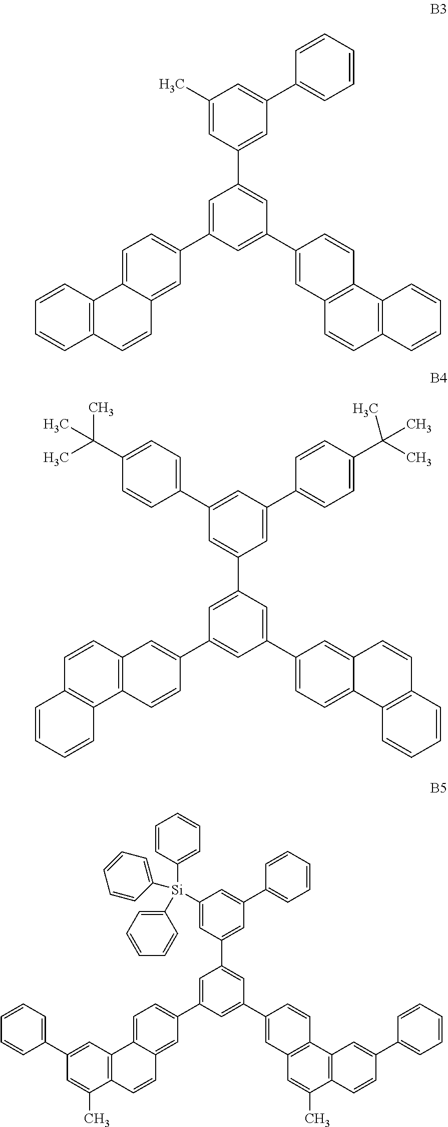

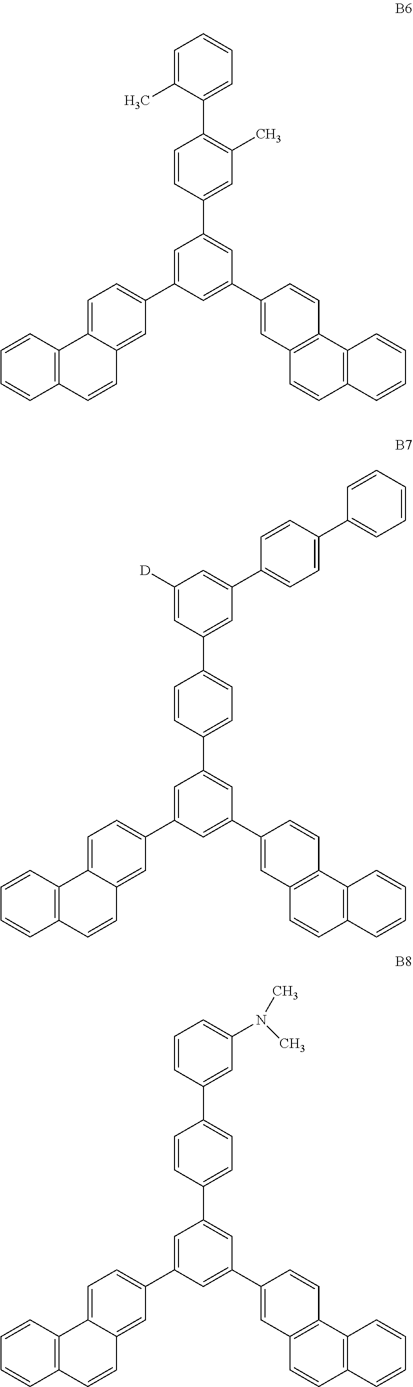

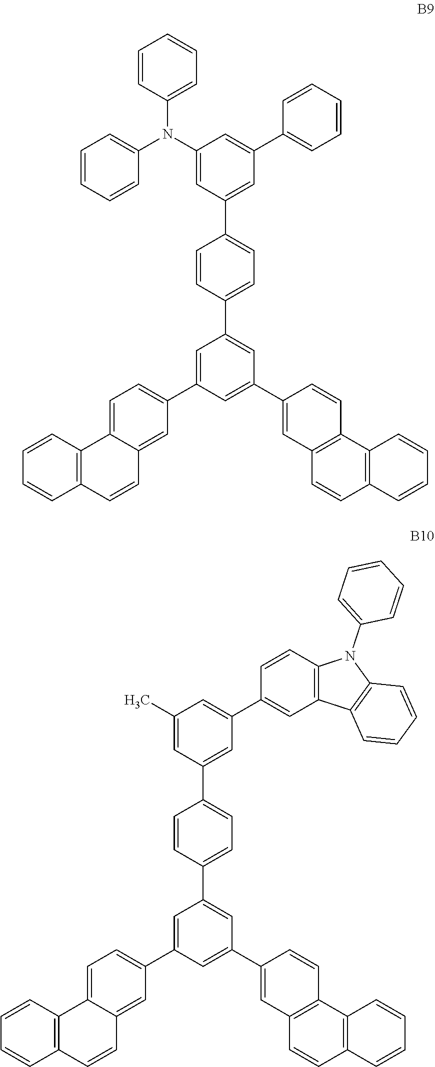 [SMILES: c1ccc(-c2cc(-c3ccc(-c4cc(-c5ccc6c(ccc7ccccc76)c5)cc(-c5ccc6c(ccc7ccccc76)c5)c4)cc3)cc(N(c3ccccc3)c3ccccc3)c2)cc1 ; Cc1cc(-c2ccc(-c3cc(-c4ccc5c(ccc6ccccc65)c4)cc(-c4ccc5c(ccc6ccccc65)c4)c3)cc2)cc(-c2ccc3c(c2)c2ccccc2n3-c2ccccc2)c1]

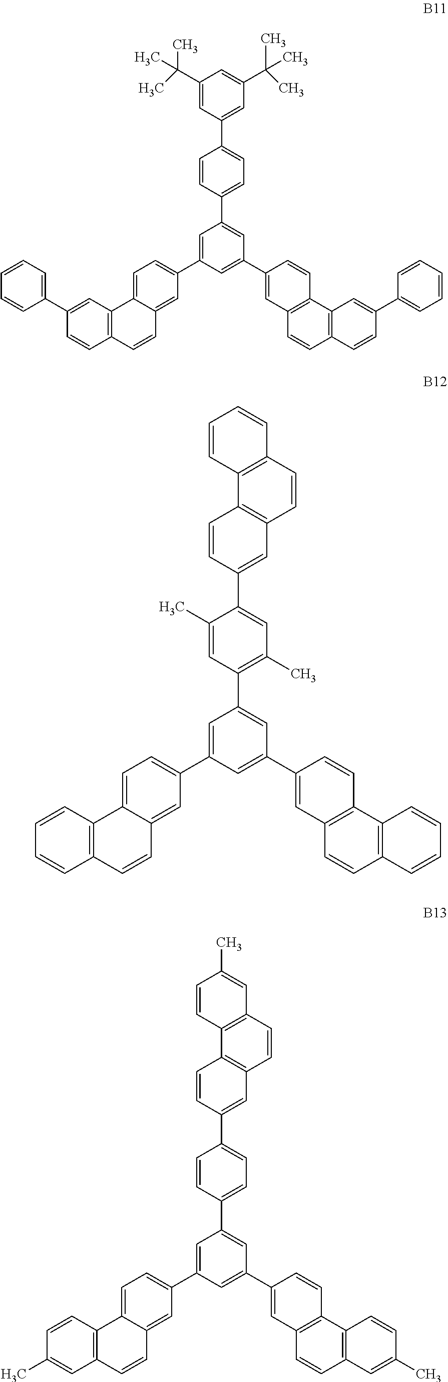

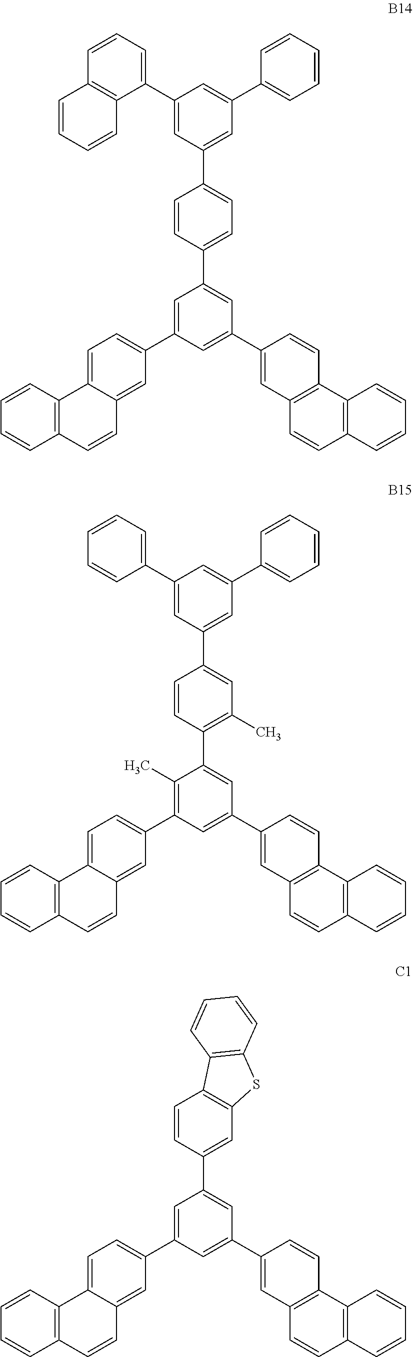

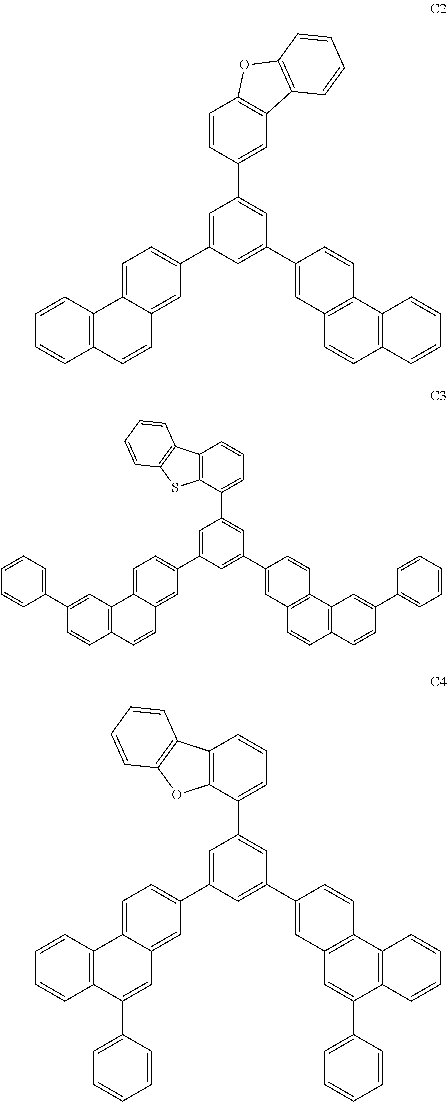 [SMILES: c1ccc2c(c1)ccc1cc(-c3cc(-c4ccc5c(ccc6ccccc65)c4)cc(-c4ccc5oc6ccccc6c5c4)c3)ccc12 ; c1ccc(-c2ccc3ccc4cc(-c5cc(-c6ccc7c(ccc8ccc(-c9ccccc9)cc87)c6)cc(-c6cccc7c6sc6ccccc67)c5)ccc4c3c2)cc1 ; c1ccc(-c2cc3cc(-c4cc(-c5ccc6c(c5)cc(-c5ccccc5)c5ccccc56)cc(-c5cccc6c5oc5ccccc56)c4)ccc3c3ccccc23)cc1]

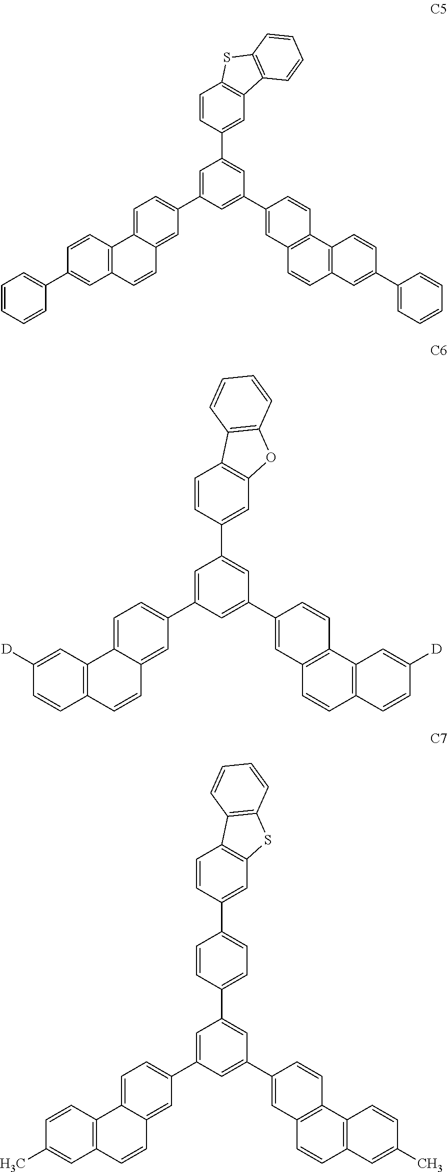

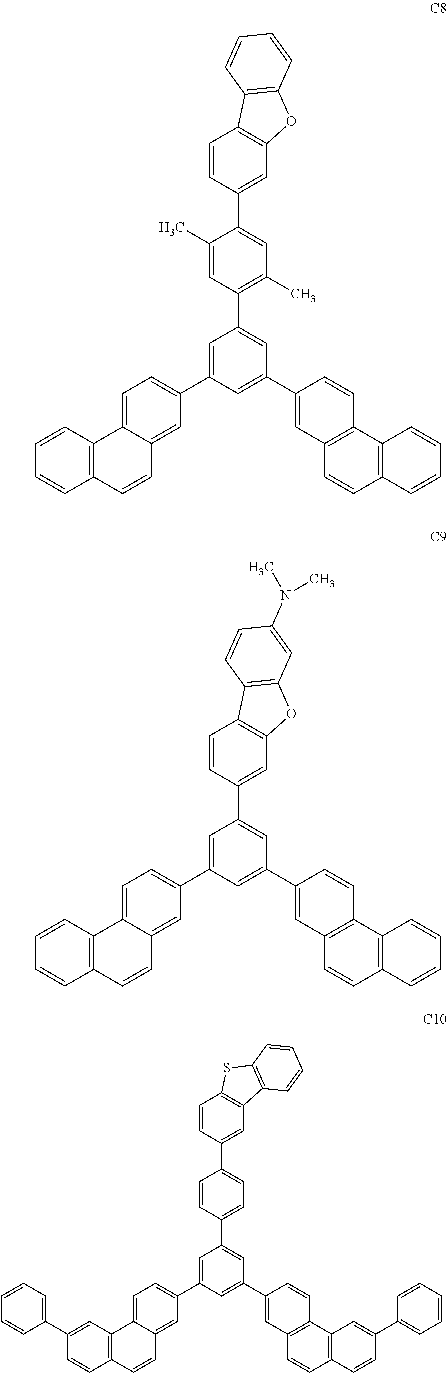 [SMILES: Cc1cc(-c2ccc3c(c2)oc2ccccc23)c(C)cc1-c1cc(-c2ccc3c(ccc4ccccc43)c2)cc(-c2ccc3c(ccc4ccccc43)c2)c1 ; CN(C)c1ccc2c(c1)oc1cc(-c3cc(-c4ccc5c(ccc6ccccc65)c4)cc(-c4ccc5c(ccc6ccccc65)c4)c3)ccc12 ; c1ccc(-c2ccc3ccc4cc(-c5cc(-c6ccc(-c7ccc8sc9ccccc9c8c7)cc6)cc(-c6ccc7c(ccc8ccc(-c9ccccc9)cc87)c6)c5)ccc4c3c2)cc1]

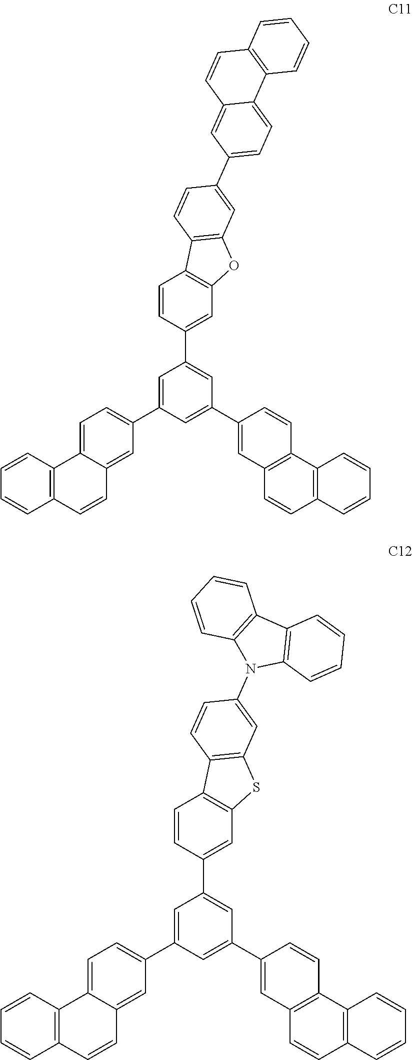 [SMILES: c1ccc2c(c1)ccc1cc(-c3cc(-c4ccc5c(ccc6ccccc65)c4)cc(-c4ccc5c(c4)oc4cc(-c6ccc7c(ccc8ccccc87)c6)ccc45)c3)ccc12 ; c1ccc2c(c1)ccc1cc(-c3cc(-c4ccc5c(ccc6ccccc65)c4)cc(-c4ccc5c(c4)sc4cc(-n6c7ccccc7c7ccccc76)ccc45)c3)ccc12]

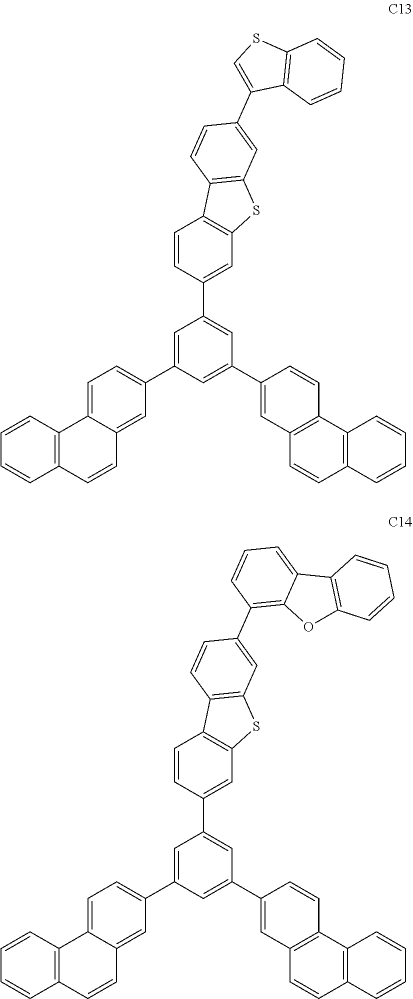 [SMILES: c1ccc2c(c1)ccc1cc(-c3cc(-c4ccc5c(ccc6ccccc65)c4)cc(-c4ccc5c(c4)sc4cc(-c6csc7ccccc67)ccc45)c3)ccc12 ; c1ccc2c(c1)ccc1cc(-c3cc(-c4ccc5c(ccc6ccccc65)c4)cc(-c4ccc5c(c4)sc4cc(-c6cccc7c6oc6ccccc67)ccc45)c3)ccc12]

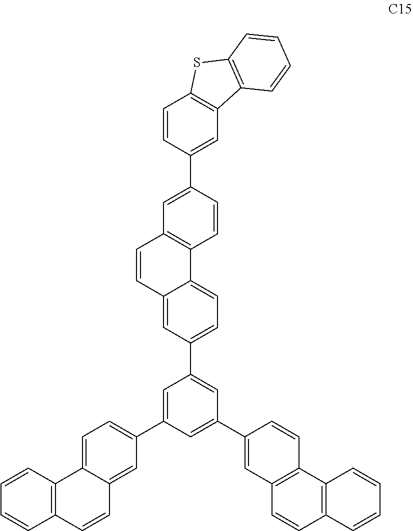 [SMILES: c1ccc2c(c1)ccc1cc(-c3cc(-c4ccc5c(ccc6ccccc65)c4)cc(-c4ccc5c(ccc6cc(-c7ccc8sc9ccccc9c8c7)ccc65)c4)c3)ccc12]

Among the exemplified compounds described above, the exemplified compounds belonging to group A are compounds having no SP3 carbon. Thus, they are organic compounds having excellent stability against holes and electrons.

Among the exemplified compounds described above, the exemplified compounds belonging to group B are compounds each having an alkyl group as a substituent. Thus, they have excellent sublimability. It is also possible to reduce the device voltage.

Among the exemplified compounds described above, the exemplified compounds belonging to group C are compounds having heteroatoms in their fused rings. Thus, they have excellent charge transport properties.

### (2) Features of Organic Light-Emitting Device

An organic light-emitting device according to an embodiment of the present disclosure includes a first electrode, an organic compound layer, and a second electrode, in this order. The organic compound layer includes at least a light-emitting layer and contains an organic compound (hereinafter, also referred to as a “host material”) represented by formula (1) and a first compound (hereinafter, also referred to as a “dopant material” or “guest material”) in the light-emitting layer. The first compound can have lower T1 than the organic compound according to an embodiment of the present disclosure.

The organic compound according to an embodiment of the present disclosure can further have the following features.

(2-1) The organic compound according to an embodiment of the present disclosure can be used in an amount of 30% to 99% by weight based on the entire light-emitting layer.
(2-2) The first compound is a phosphorescent material having a fused ring as a ligand and thus is excellent in luminous efficiency.

These features are described below.

(2-1) The organic compound according to an embodiment of the present disclosure can be used in an amount of 30% to 99% by weight based on the entire light-emitting layer.

The organic compound according to an embodiment of the present disclosure is highly amorphous. Thus, when the organic compound according to an embodiment of the present disclosure is used in the light-emitting layer, the organic compound according to an embodiment of the present disclosure can be used in an amount of 30% to 99% by weight. When the organic compound according to an embodiment of the present disclosure is used as a host material, the organic compound according to an embodiment of the present disclosure can be used in an amount of 50% by weight or more. Moreover, the organic compound according to an embodiment of the present disclosure is a material that is difficult to crystallize even when used in an amount of 99% by weight, and thus is a compound excellent in luminous efficiency and durability. This is due to the structural feature of the organic compound according to an embodiment of the present disclosure. The substitution of two phenanthrenyl groups and an aryl group around the benzene results in a high glass transition temperature, so that the compound is resistant to aggregation. Even when the organic light-emitting device is driven, crystal grain boundaries due to molecular aggregation are not easily formed. It is thus possible to provide a light-emitting device excellent in luminous efficiency and device lifetime.

The organic compound according to an embodiment of the present disclosure may be used as an assist material. The assist material complements the carrier transport properties of the host material and has a role of promoting the injection and transfer of carriers into the light-emitting layer. When the organic compound according to an embodiment of the present disclosure is used as an assist material, the organic compound according to an embodiment of the present disclosure can be used in an amount of 30% to 50% by weight.

(2-2) The first compound is a phosphorescent material having a fused ring as a ligand and thus is excellent in luminous efficiency.

The organic compound according to an embodiment of the present disclosure is a compound containing three or more fused rings attached to benzene. Thus, the phosphorescent material used together with the organic compound according to an embodiment of the present disclosure in the light-emitting layer can contain a fused ring with a structure in which the it conjugation of the ligand is extended, or can contain a fused ring composed of at least three or more rings. Specifically, the phosphorescent material can contain an aryl group having 12 to 20 carbon atoms or a heterocyclic group having 12 to 18 carbon atoms. More specifically, the phosphorescent material can contain a phenanthrene skeleton, a triphenylene skeleton, a dibenzofuran skeleton, a dibenzothiophene skeleton, a fluorene skeleton, a benzofluorene skeleton, a naphthoisoquinoline skeleton, or a benzoisoquinoline skeleton, or can contain a phenanthrene skeleton, a benzofluorene skeleton, a dibenzofuran skeleton, a triphenylene skeleton, a naphthoisoquinoline skeleton, or a benzoisoquinoline skeleton.

When the first compound has the above structure, the first compound has a highly planar structure like the host material. Thus, the fused rings of the first compound and the host material can approach each other through interaction. Specifically, the fused ring moiety of the host material and the ligand of the first compound are more likely to approach each other. Thus, the intermolecular distance between the host material and the first compound should be shortened.

It is known that in the triplet energy used in phosphorescent devices, energy transfer occurs by the Dexter mechanism. In the Dexter mechanism, energy transfer occurs through contact between molecules. That is, the intermolecular distance between the host material and the guest material is decreased, thereby resulting in efficient energy transfer from the host material to the guest material.

In an embodiment of the present disclosure, the intermolecular distance of the host material, which is the organic compound according to an embodiment of the present disclosure, is shortened by using a highly planar phosphorescent material having a fused ring in the ligand structure. Thus, energy transfer from the host to the first compound is likely to occur. As a result, an organic light-emitting device with excellent luminous efficiency can be provided.

Specific examples of the first compound according to an embodiment of the present disclosure are illustrated below. However, the invention is not limited thereto.

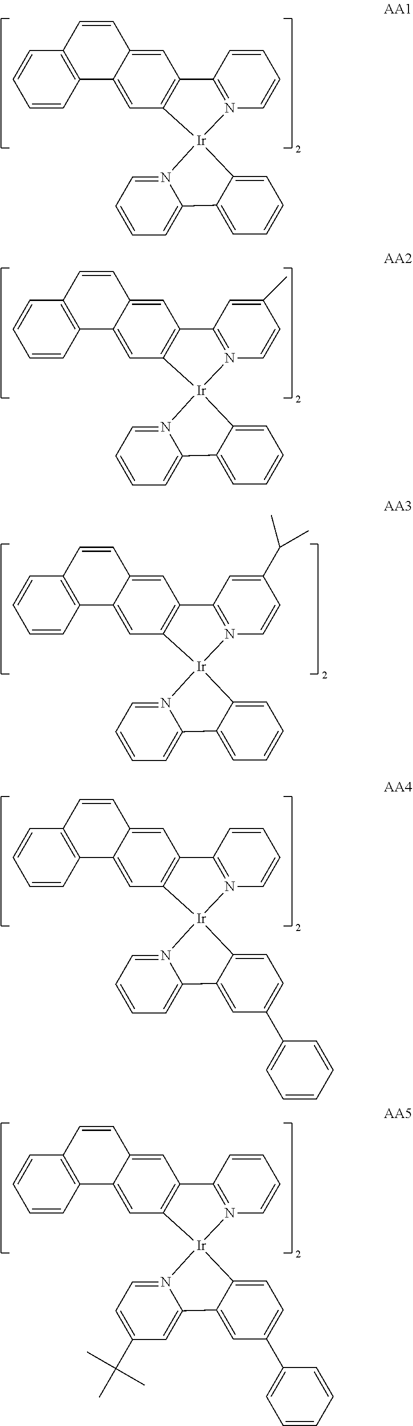

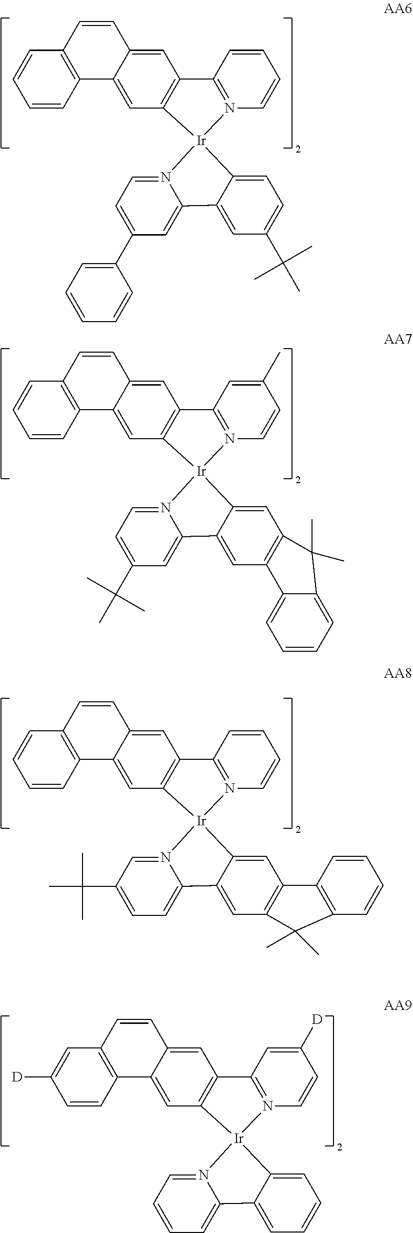

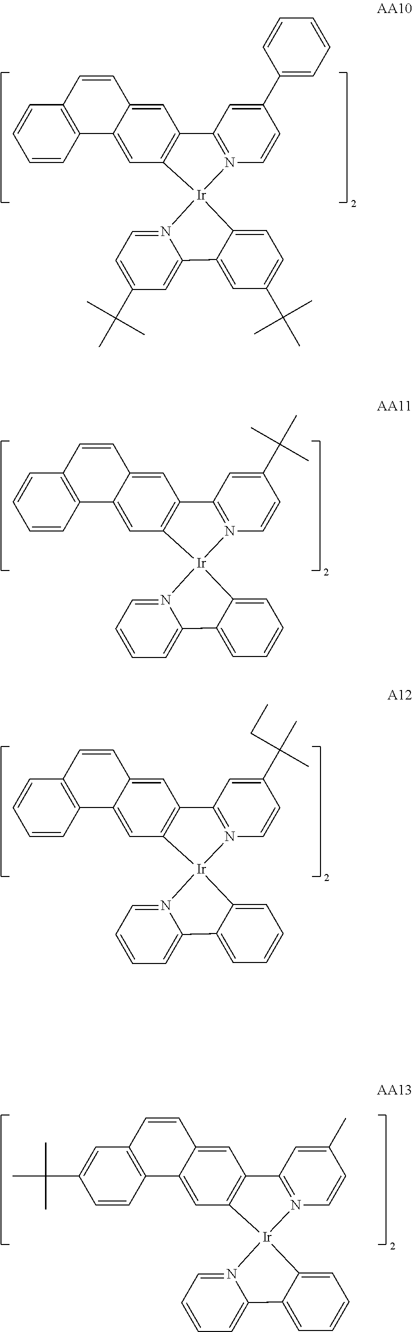

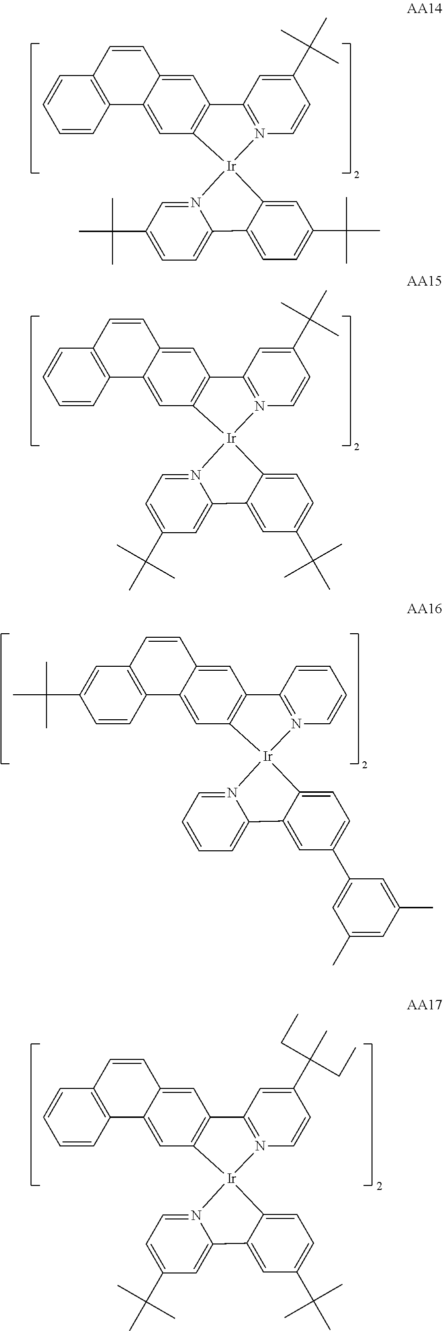

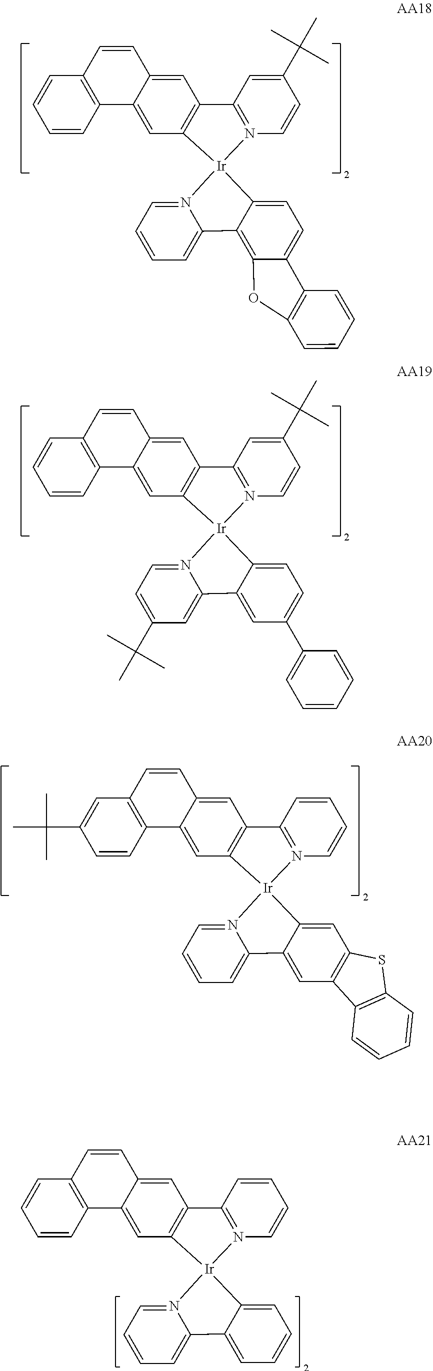

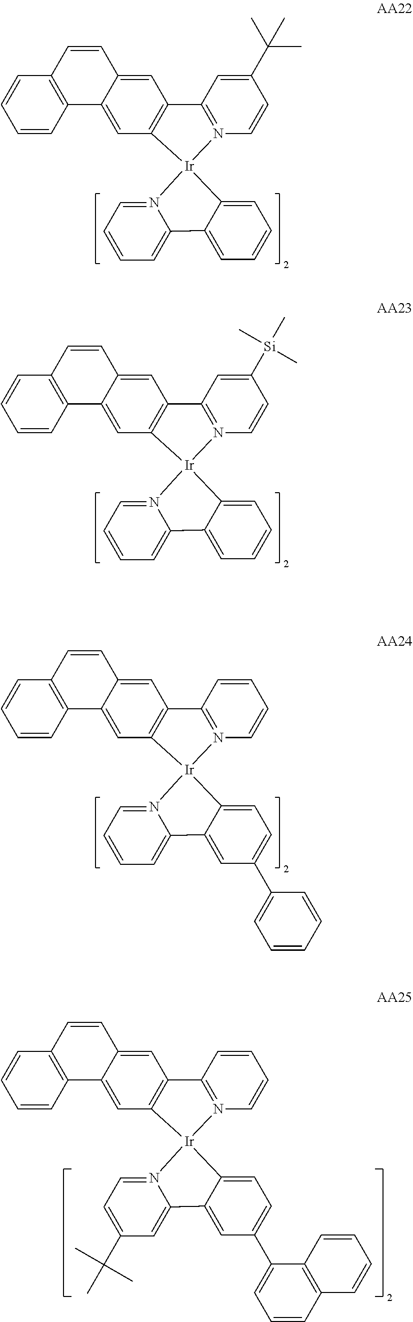

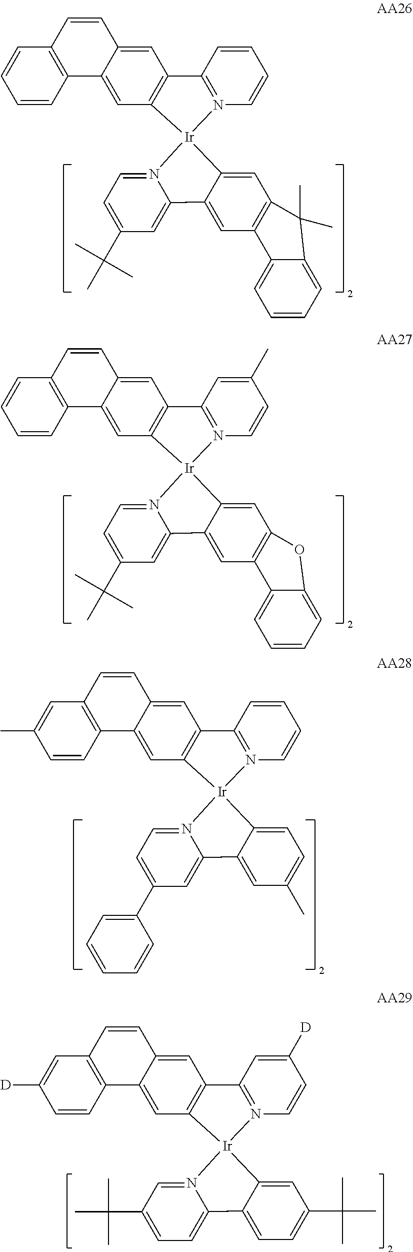

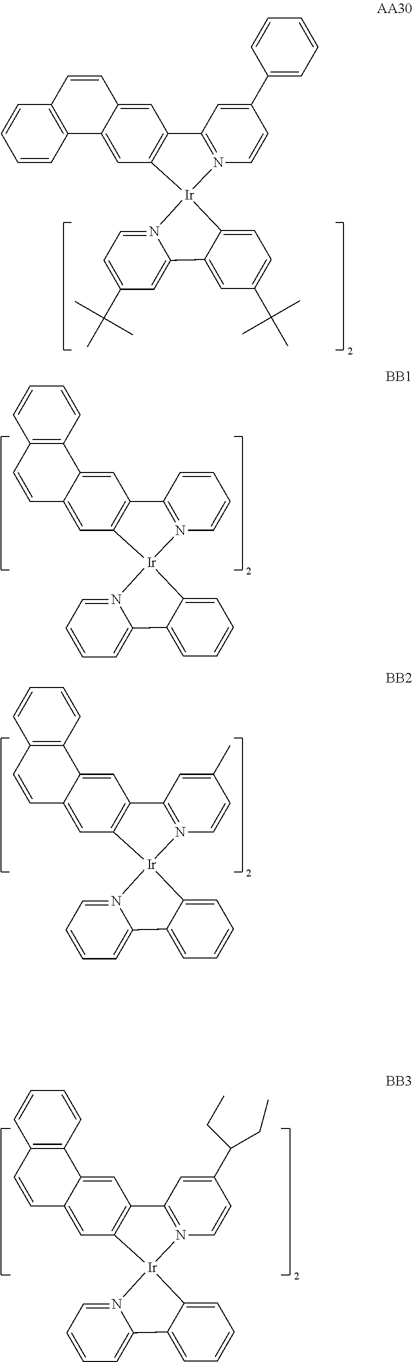

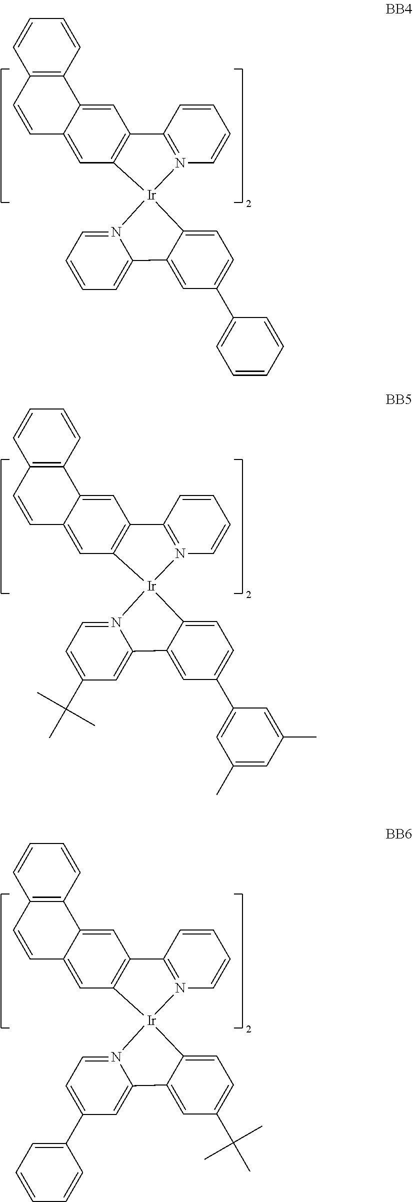

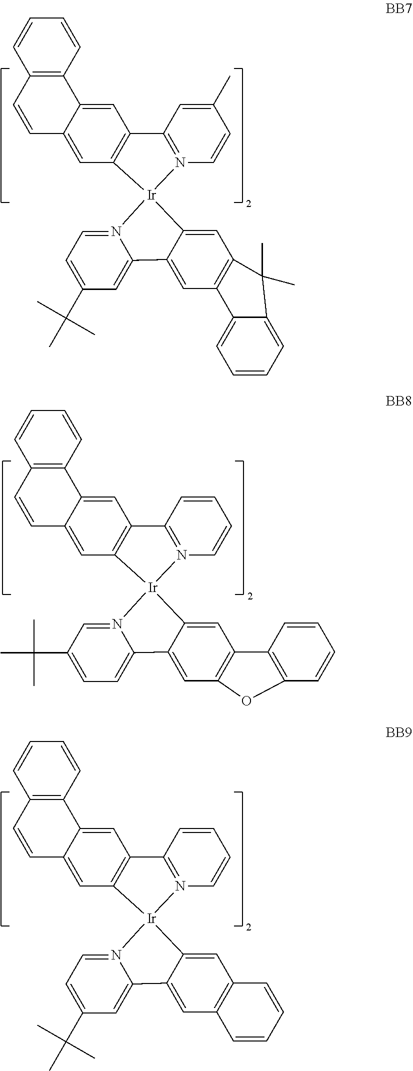

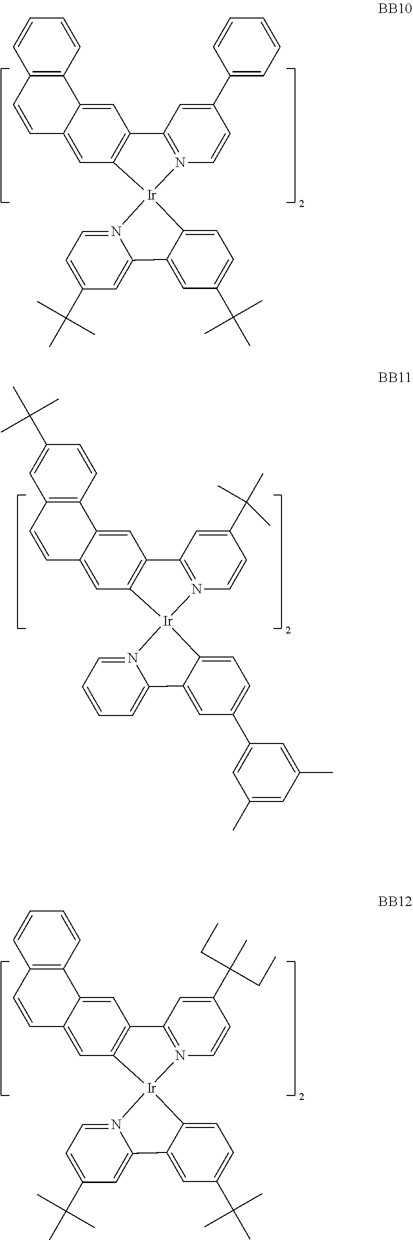

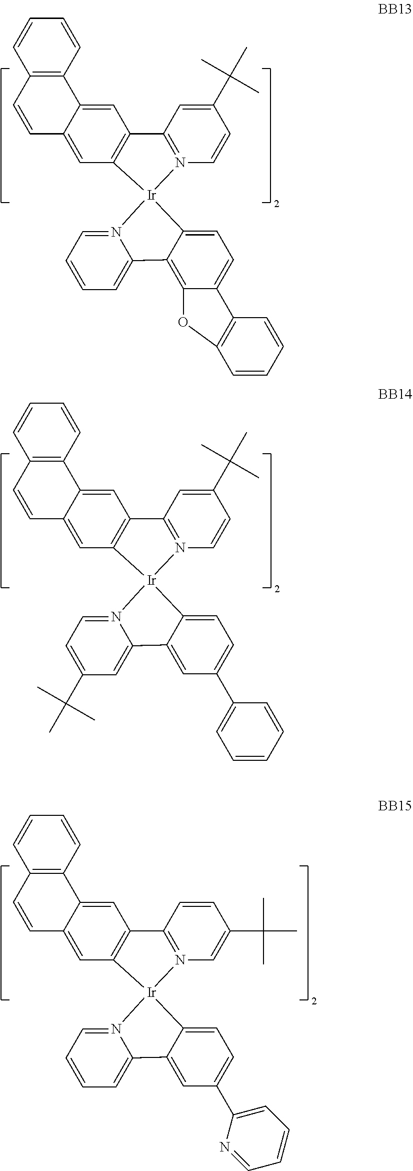

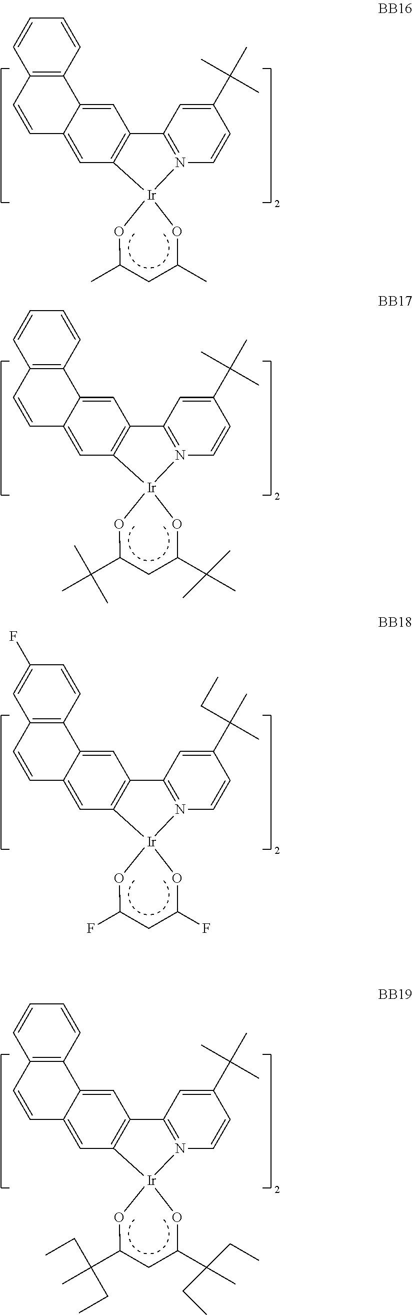

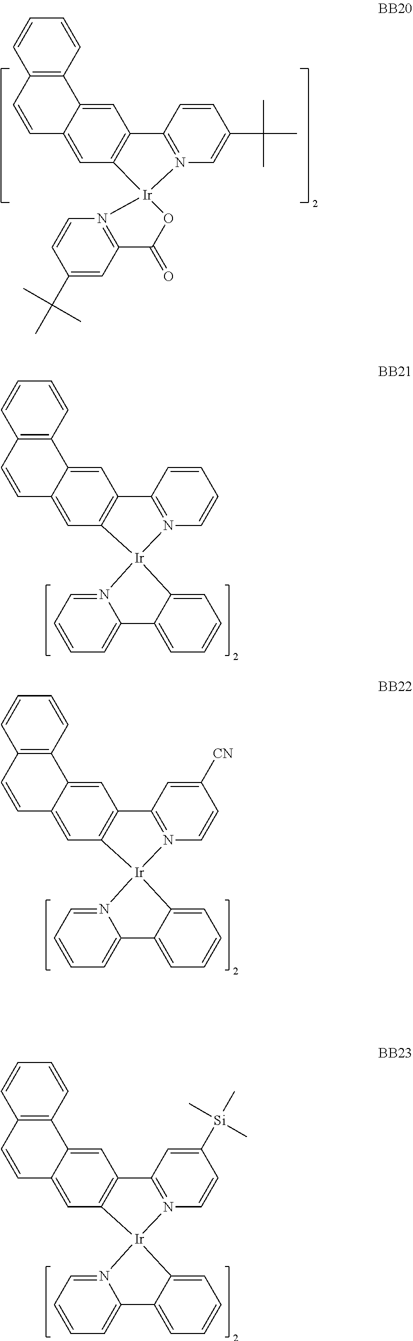

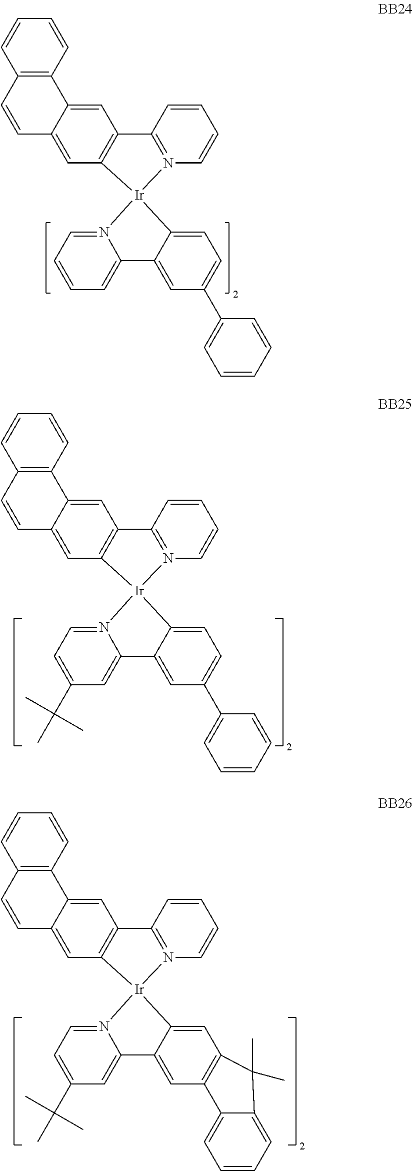

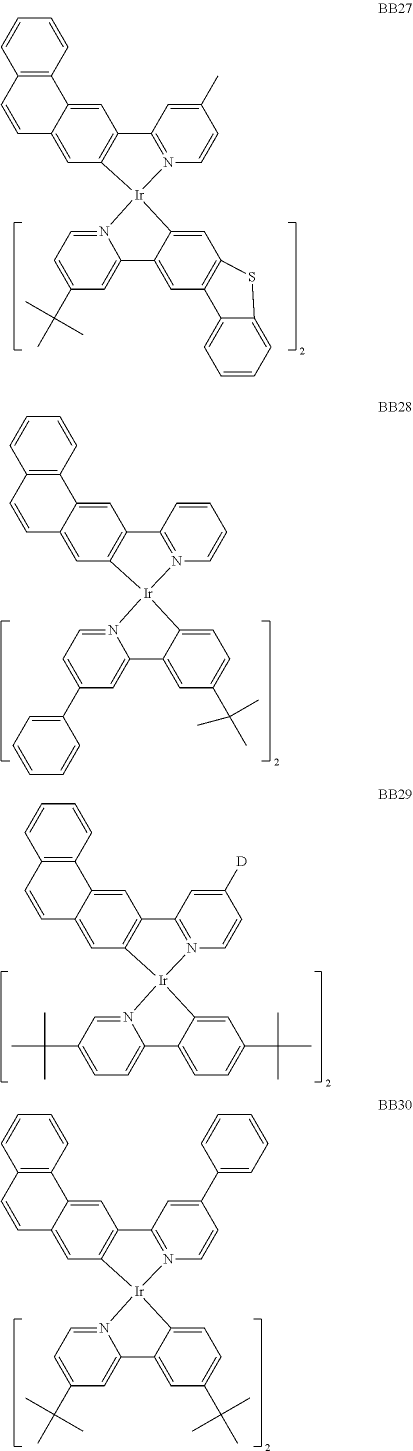

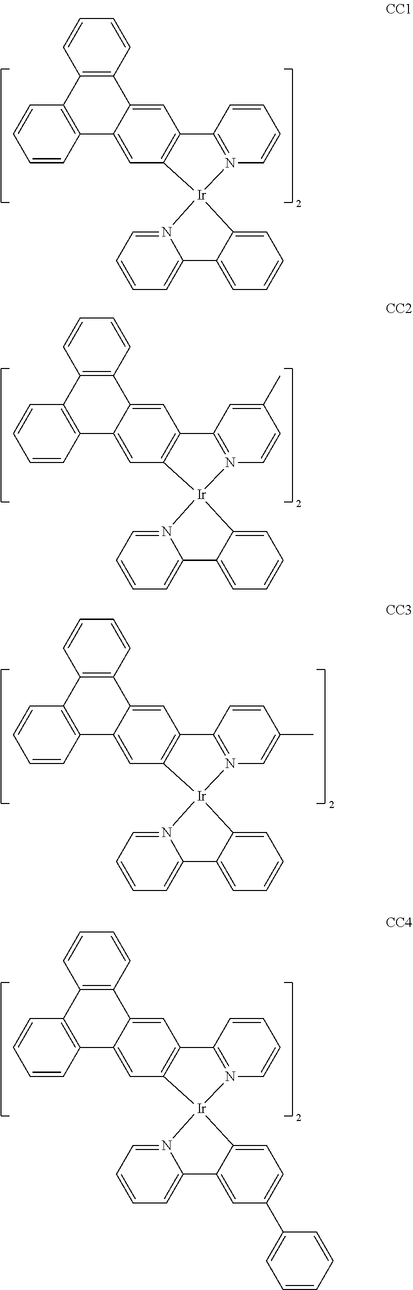

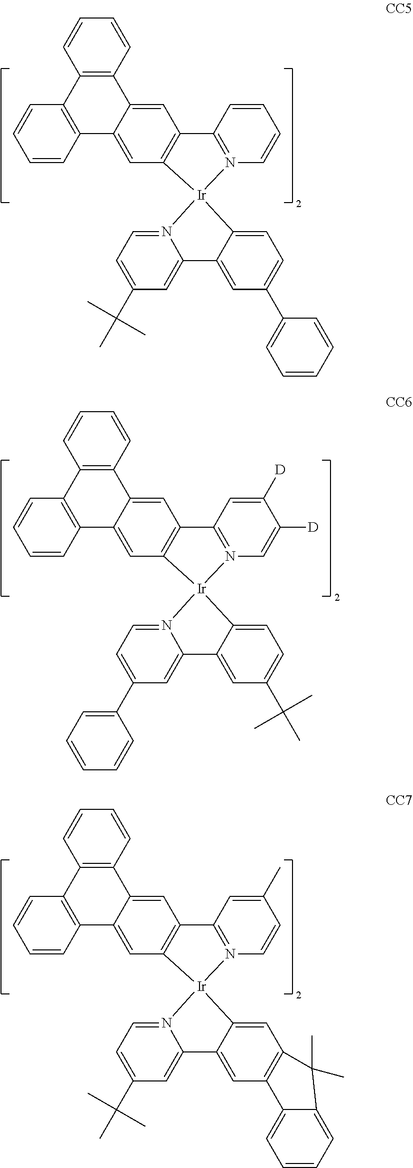

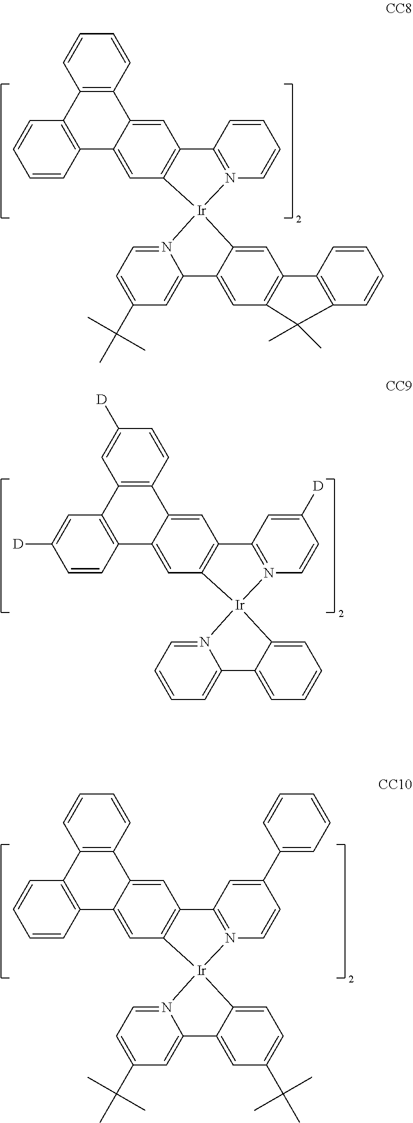

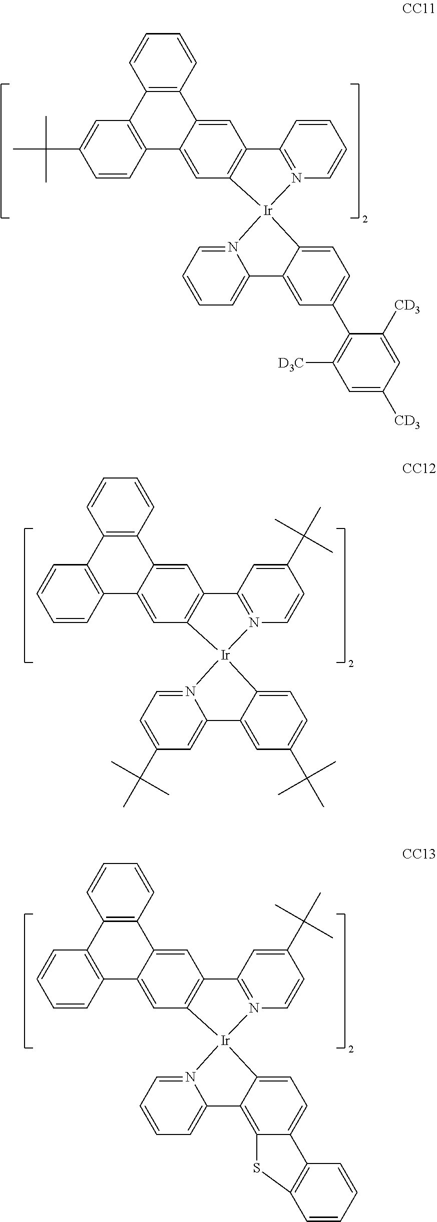

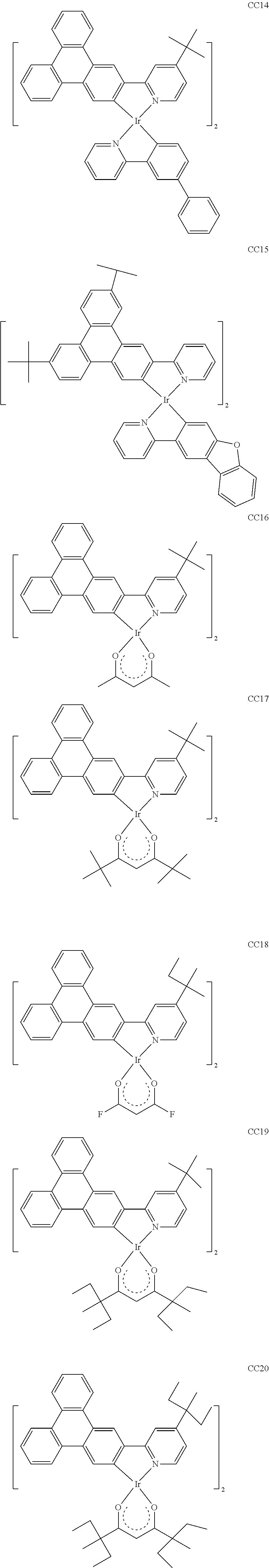

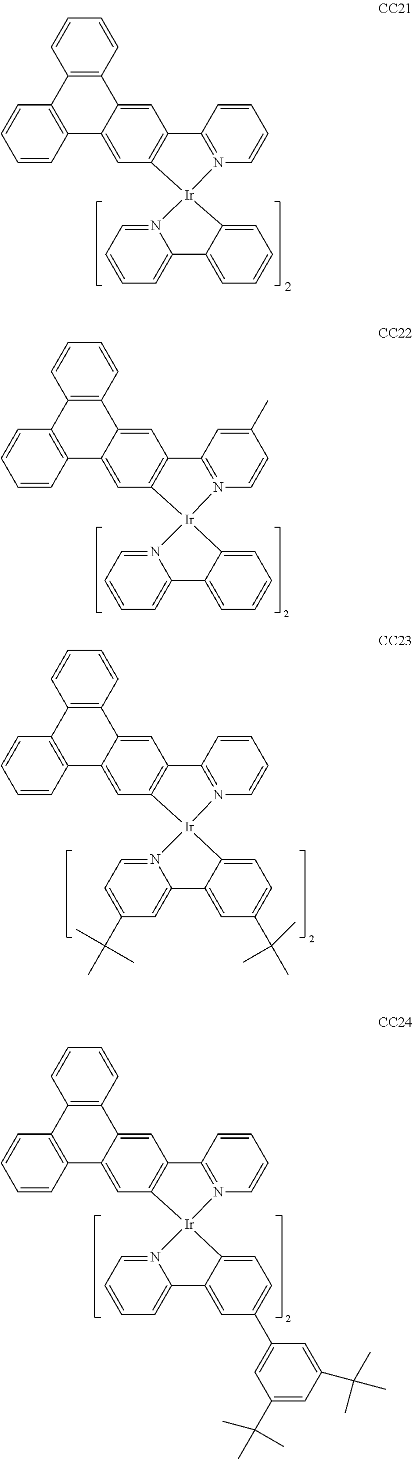

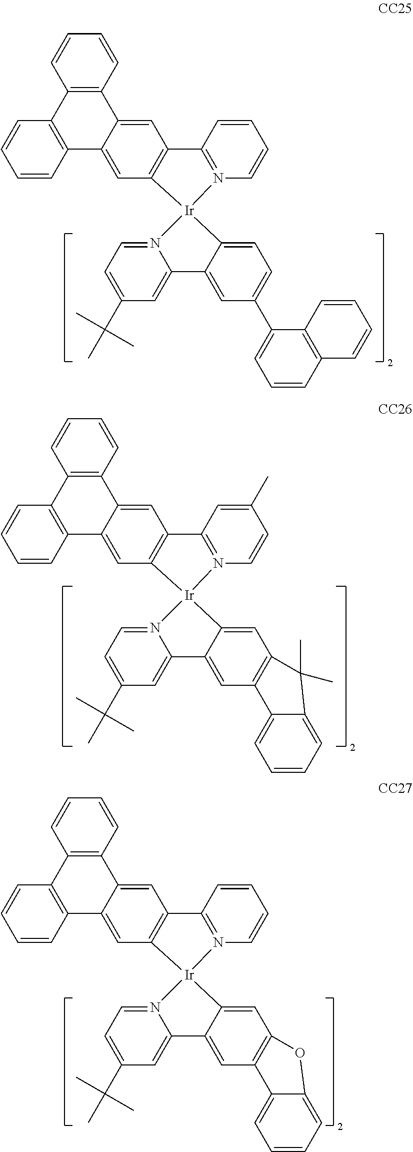

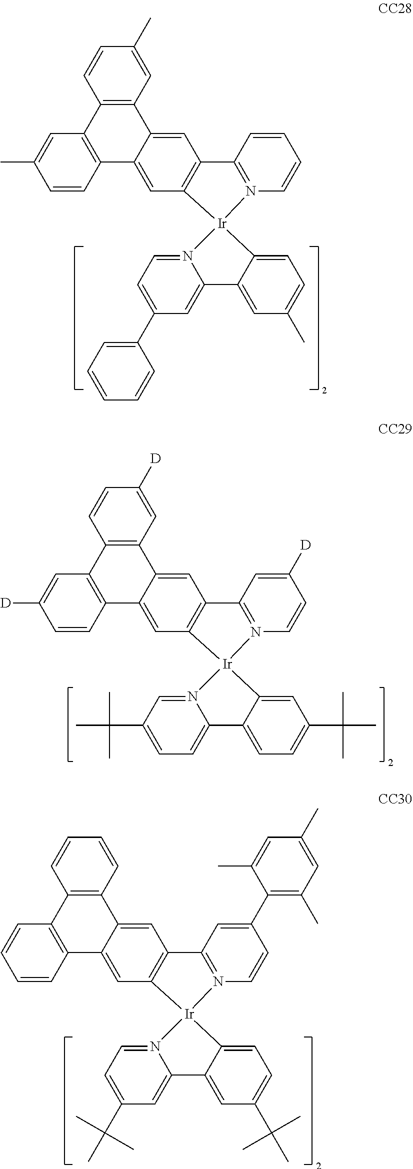

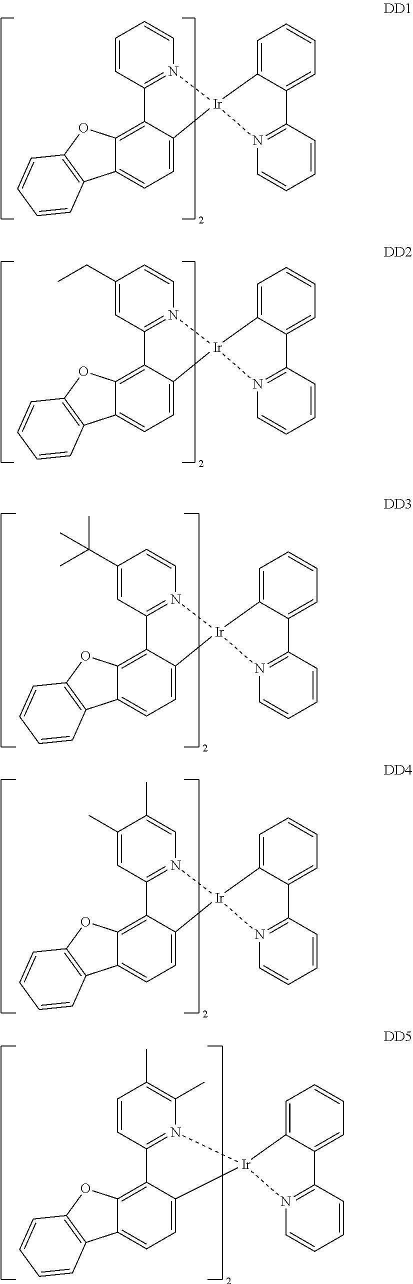

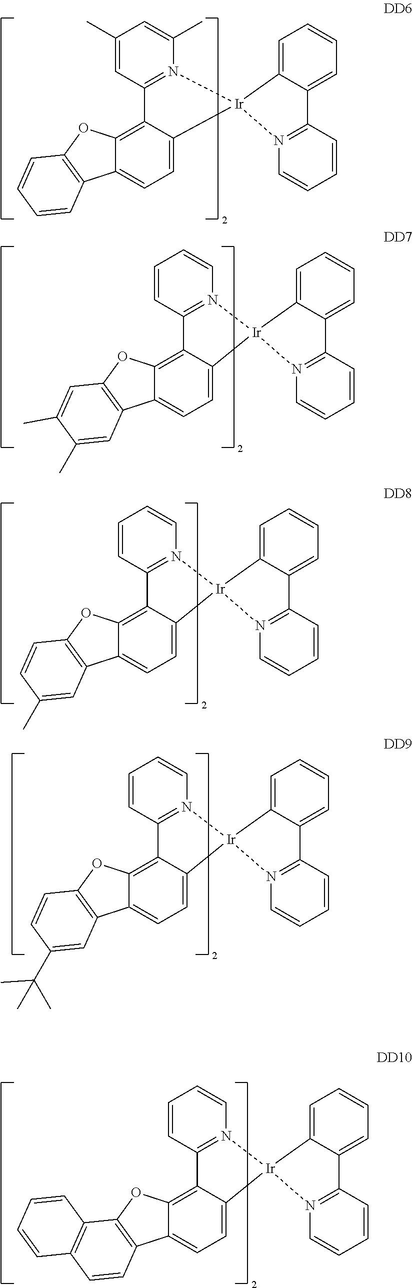

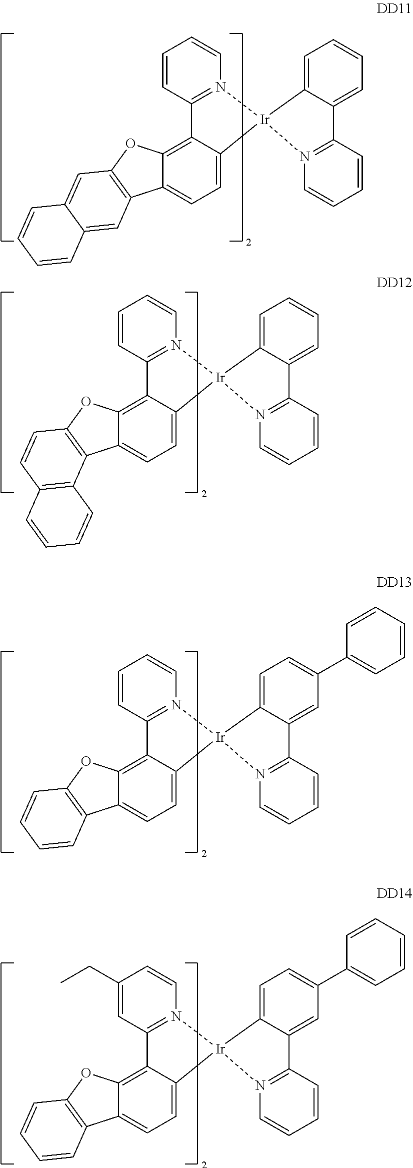

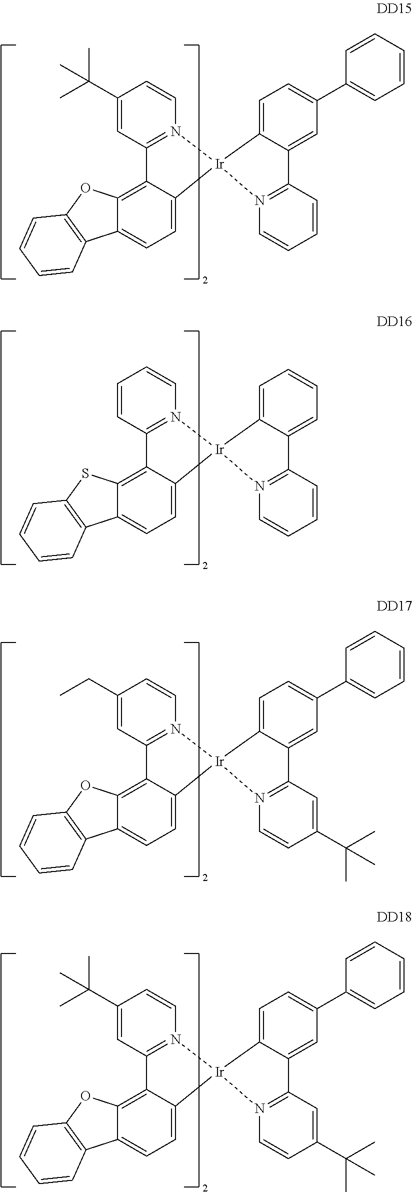

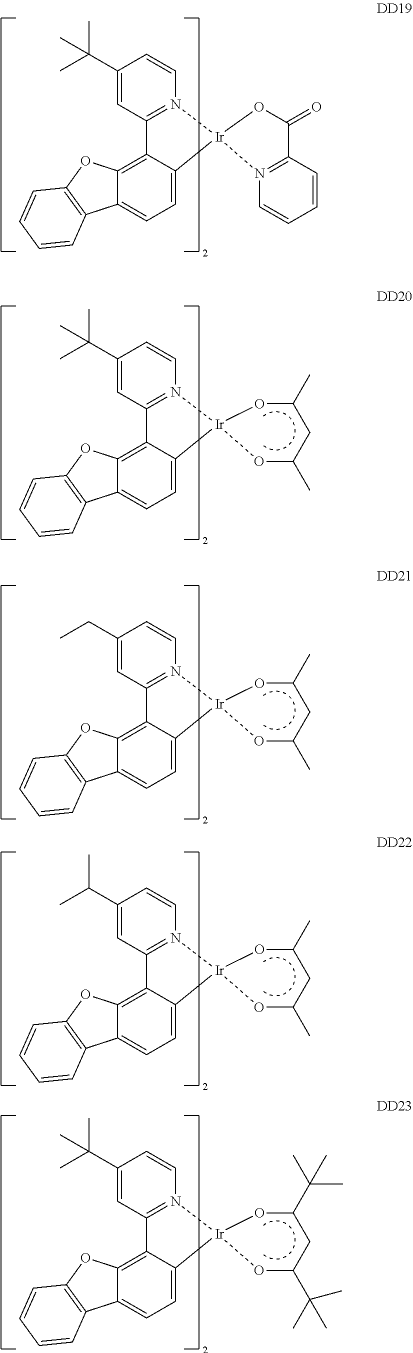

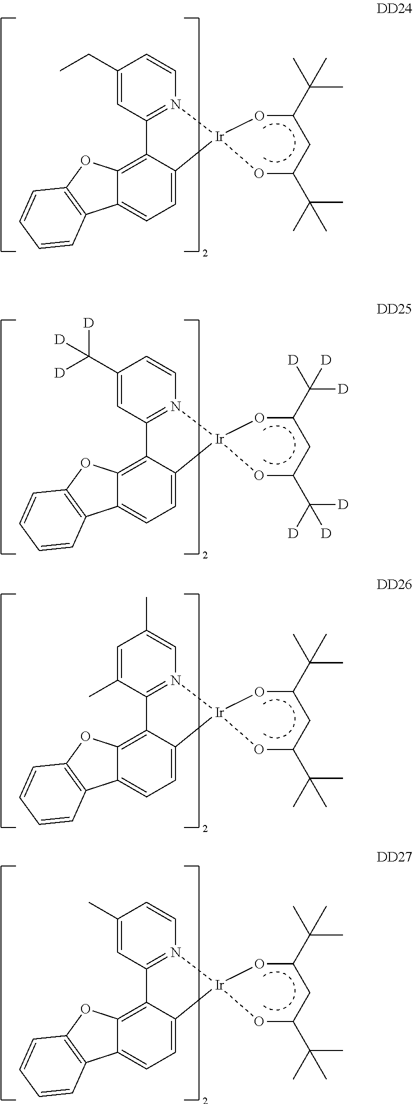

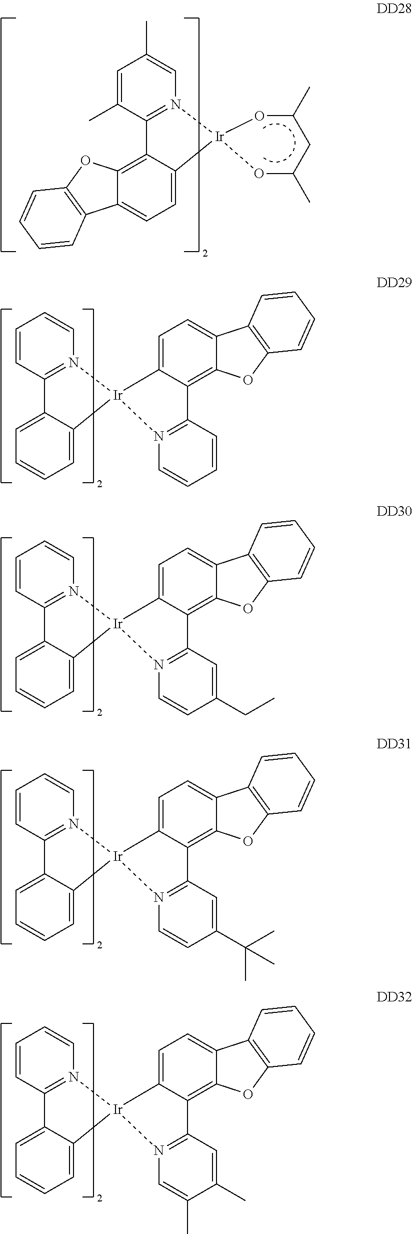

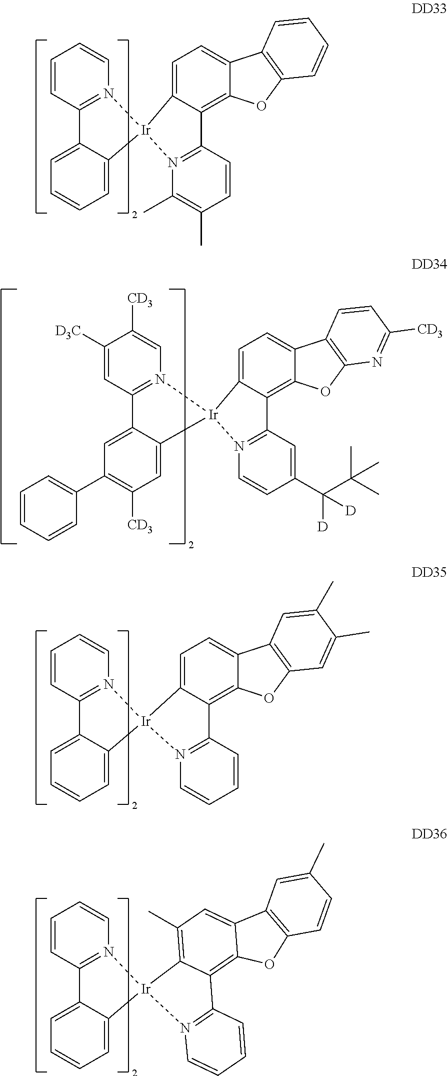

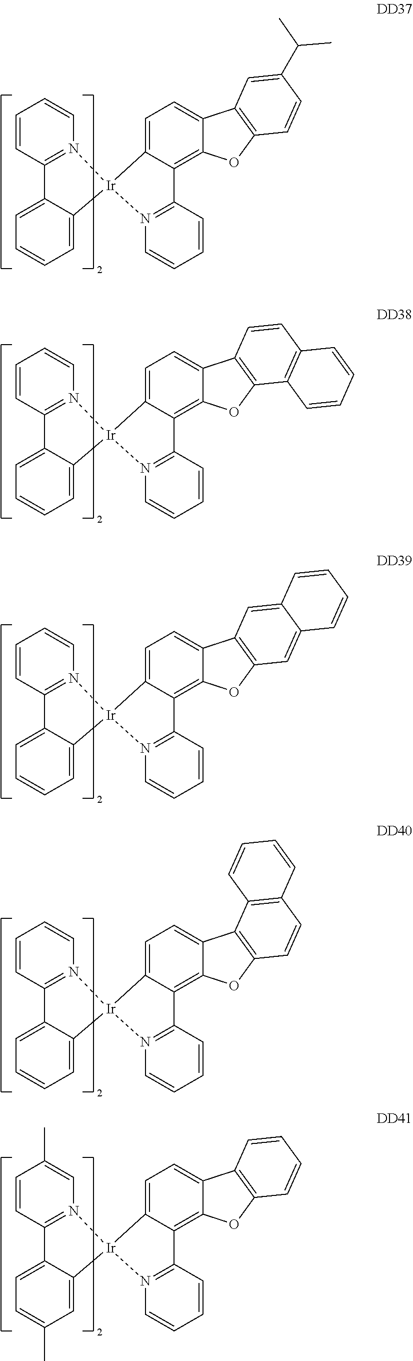

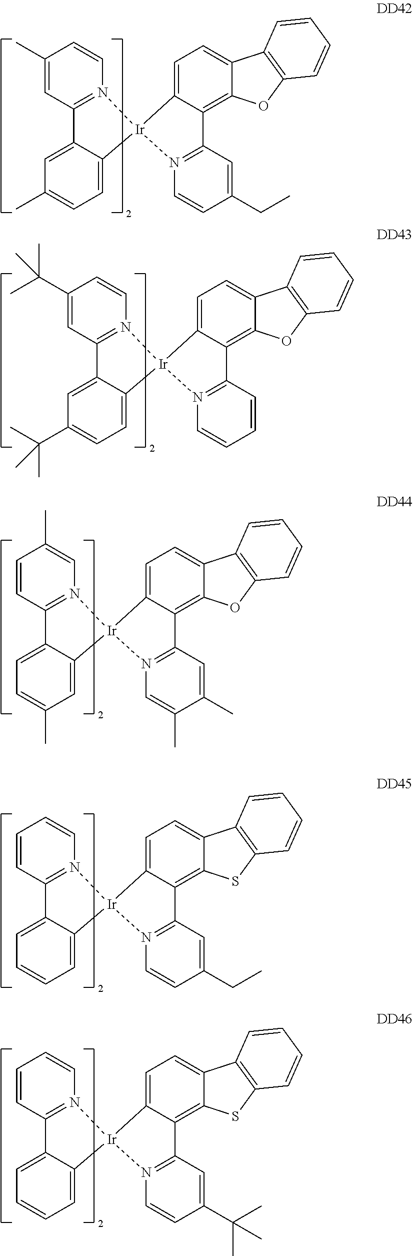

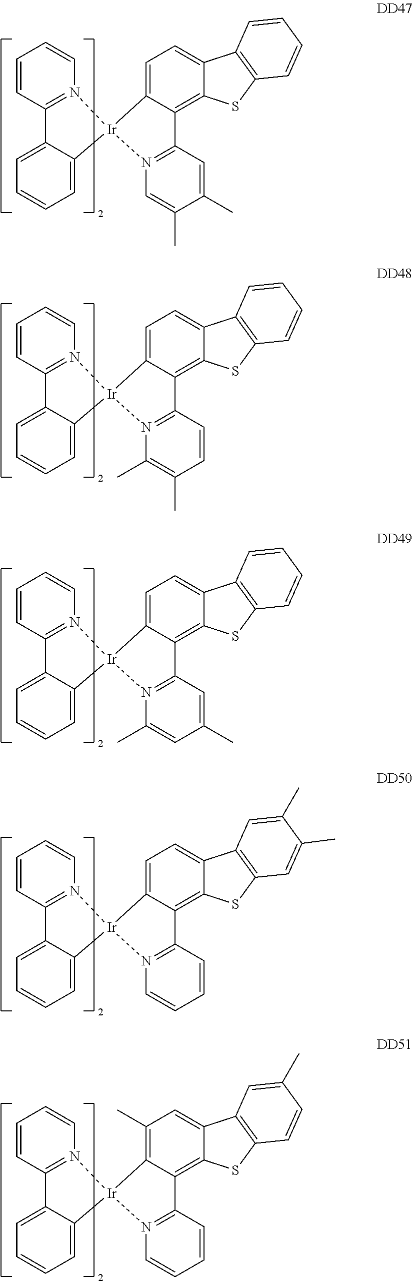

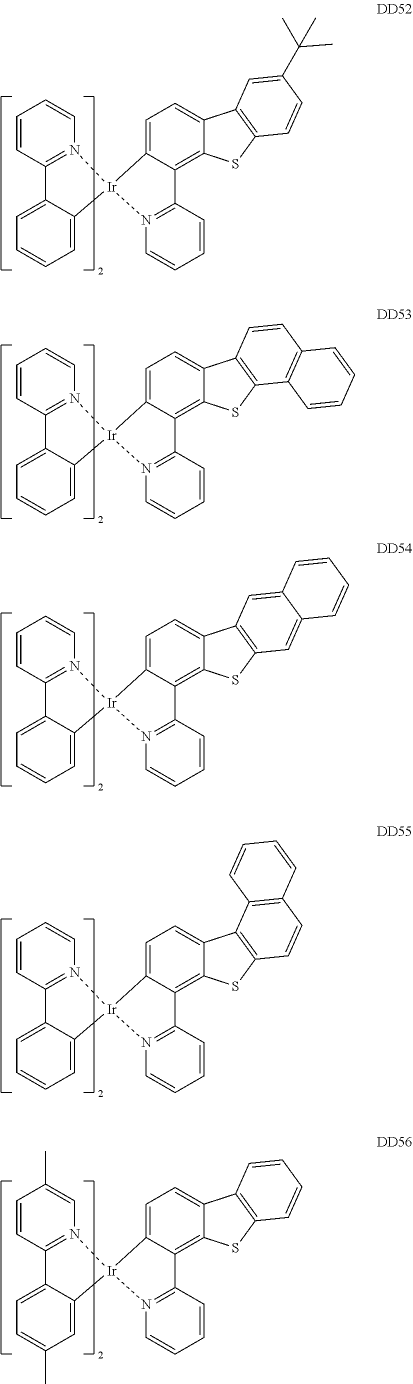

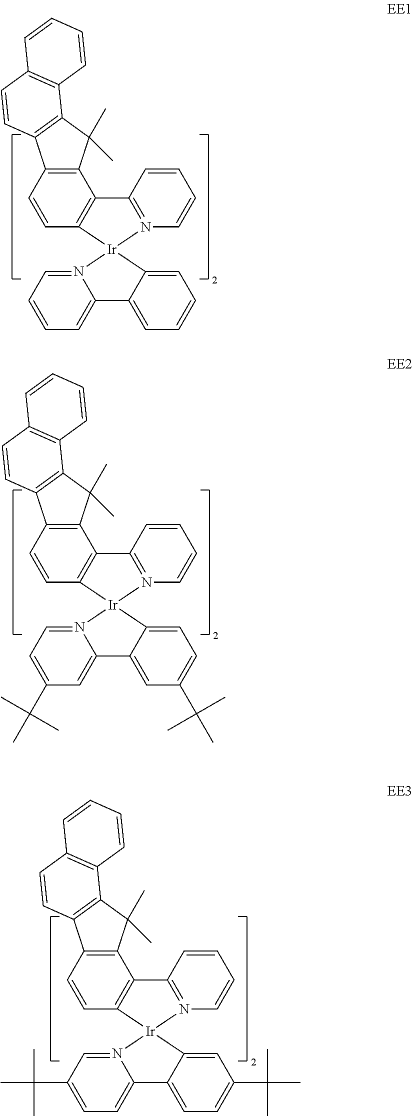

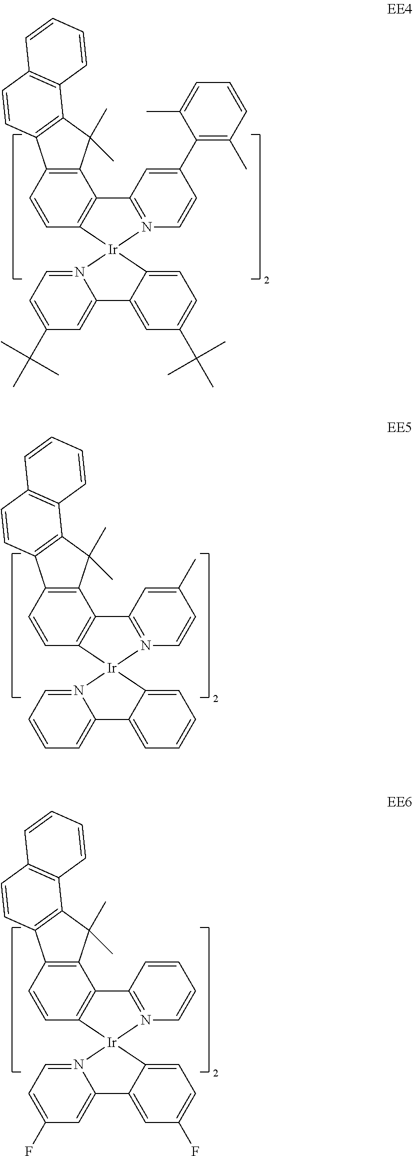

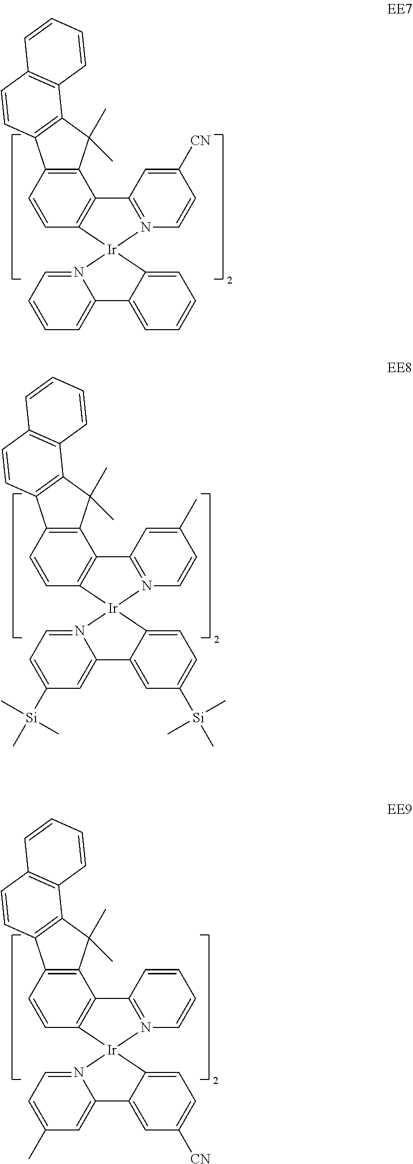

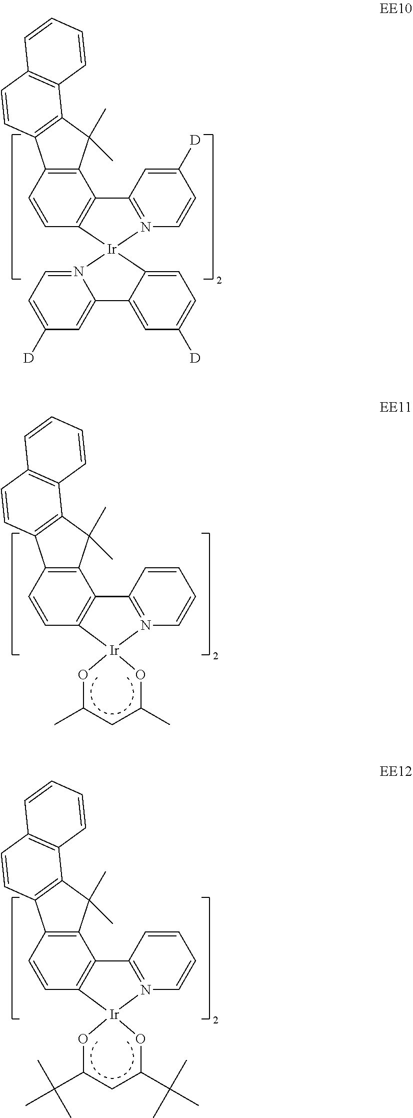

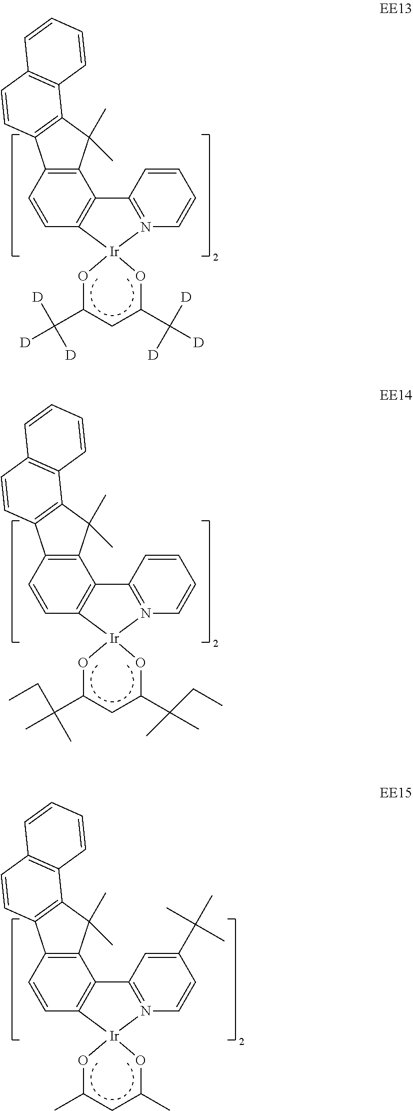

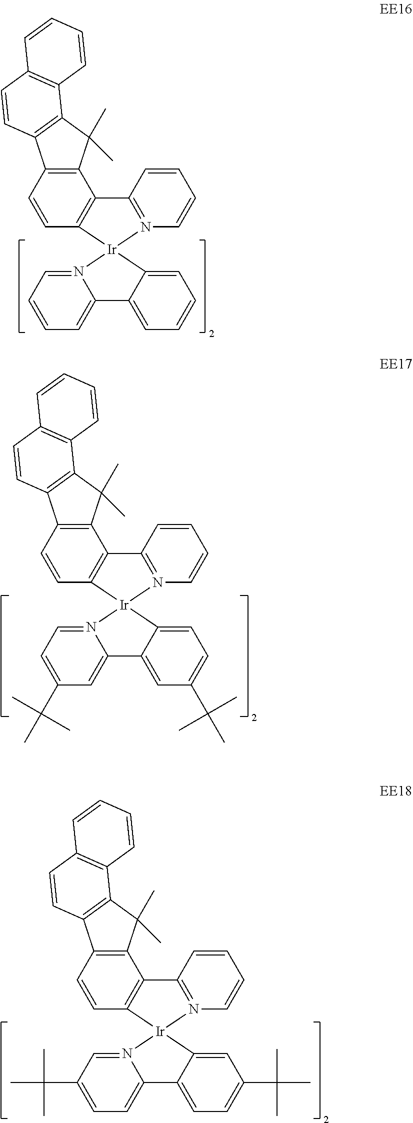

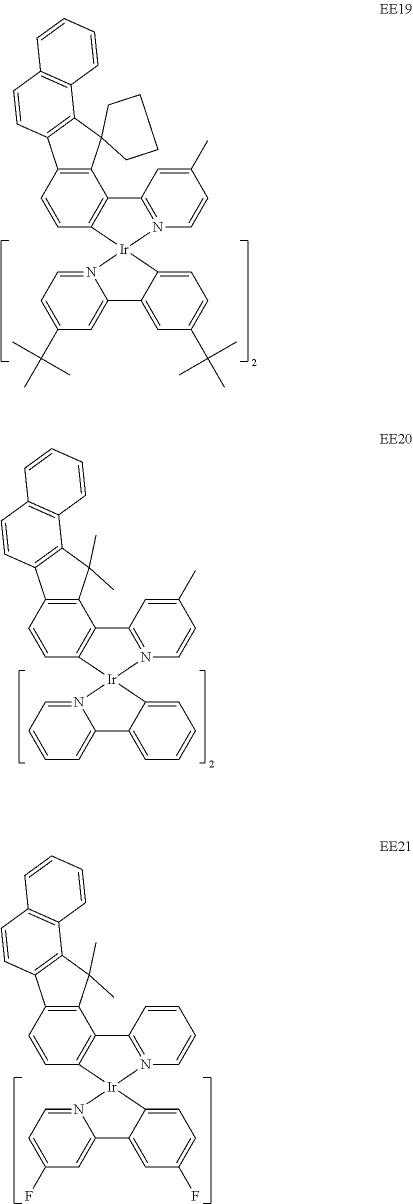

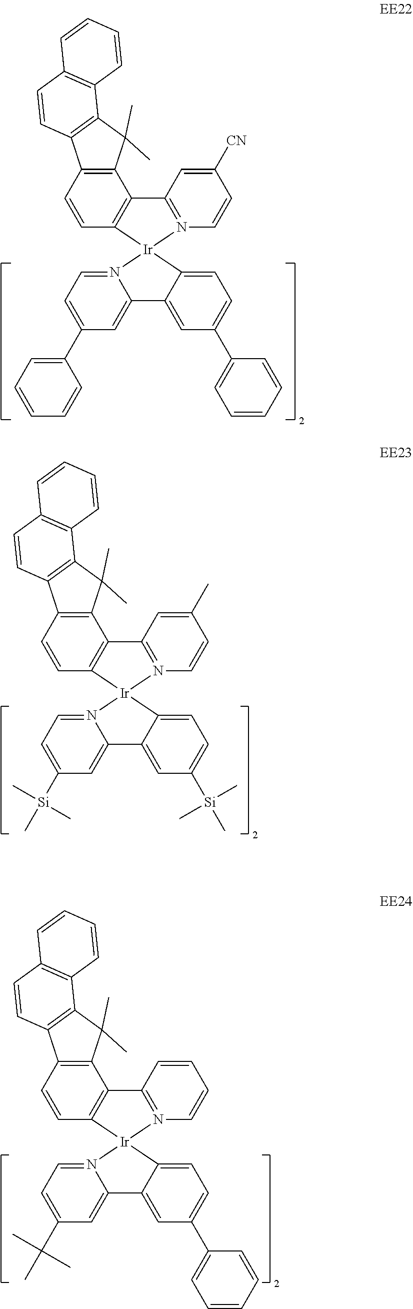

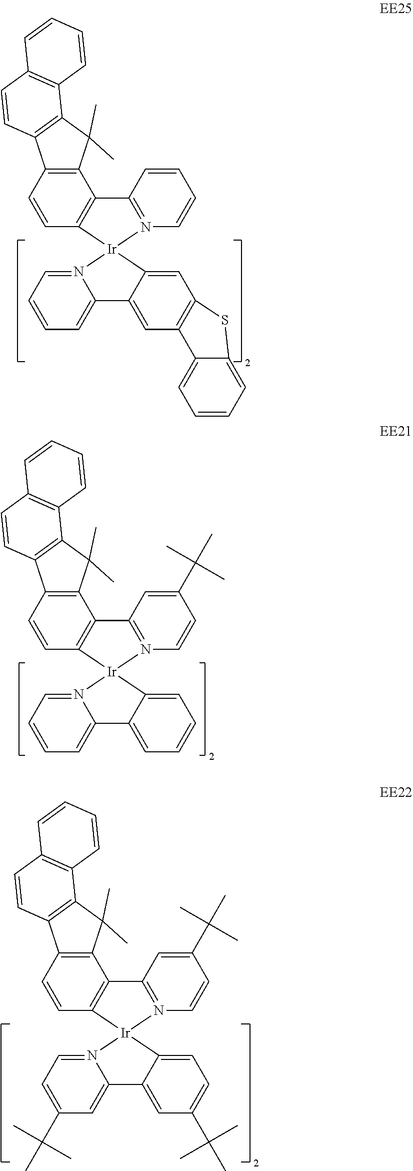

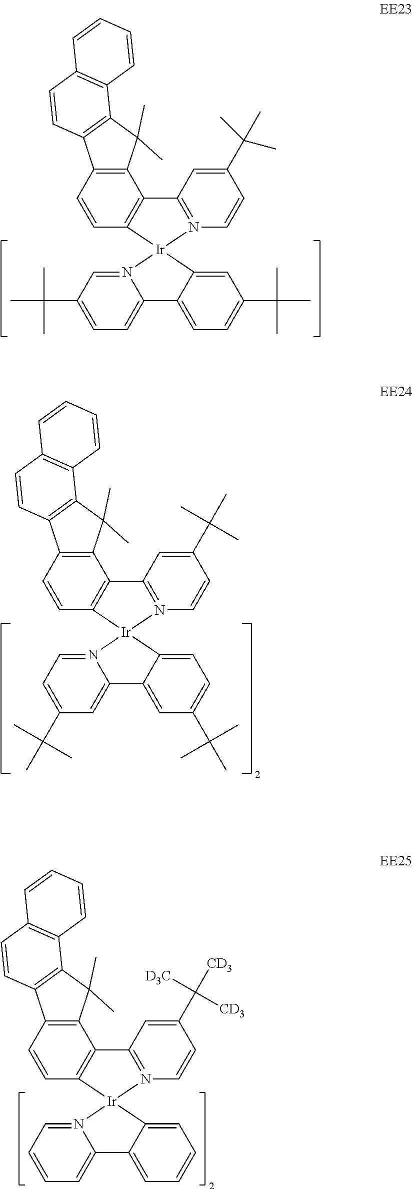

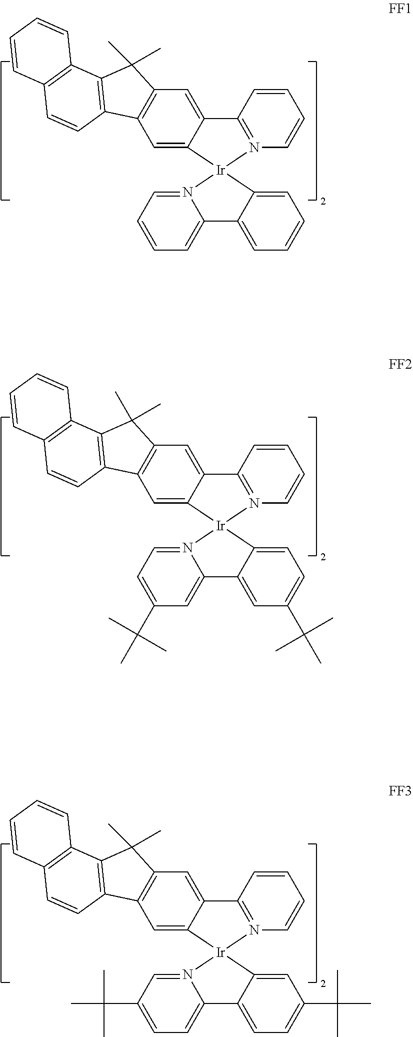

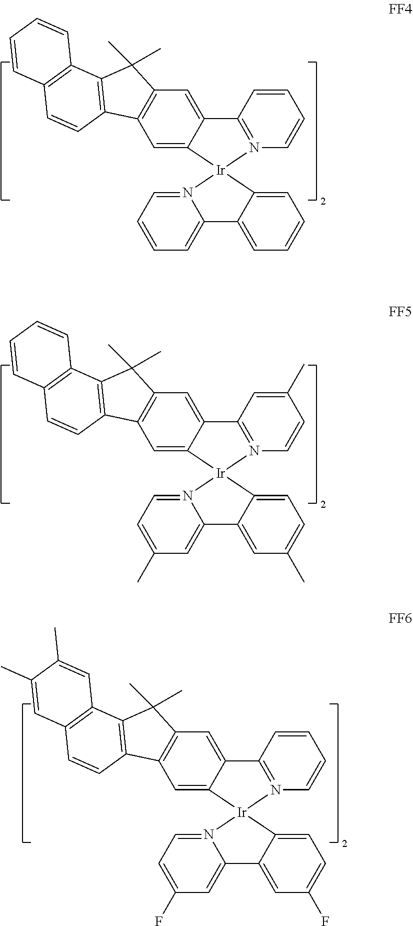

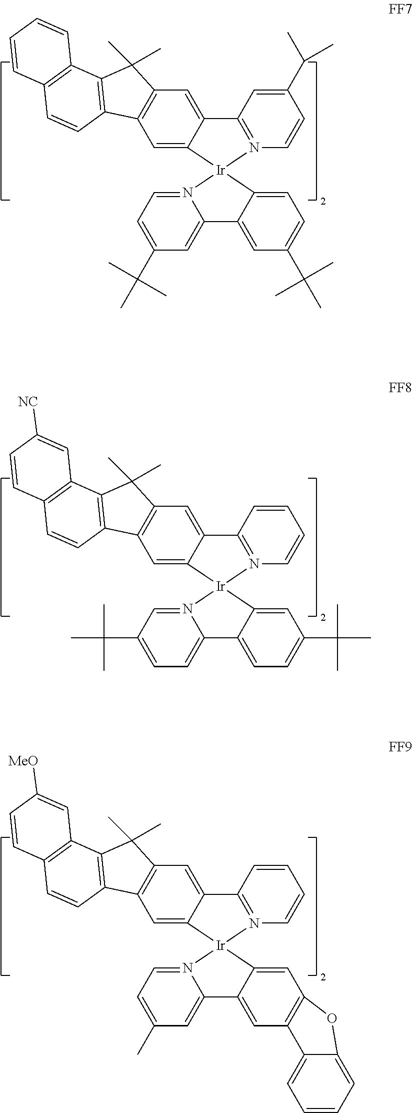

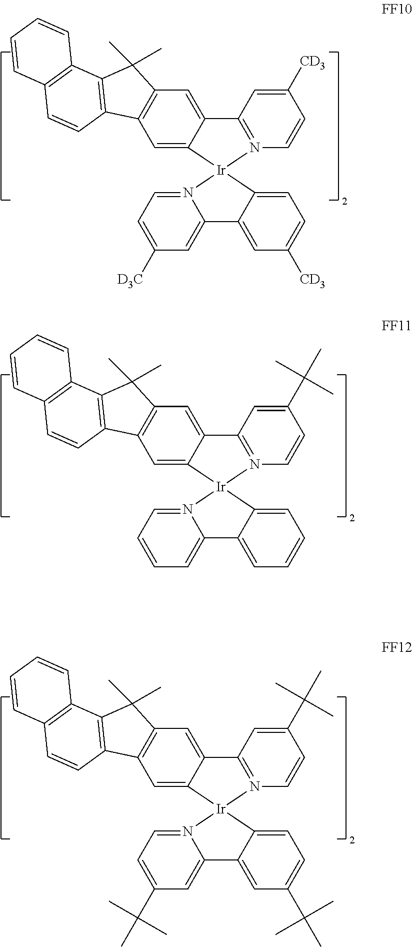

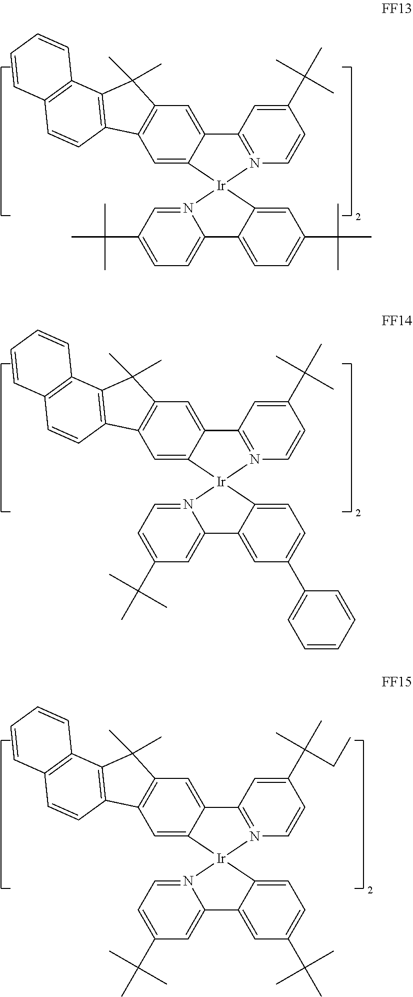

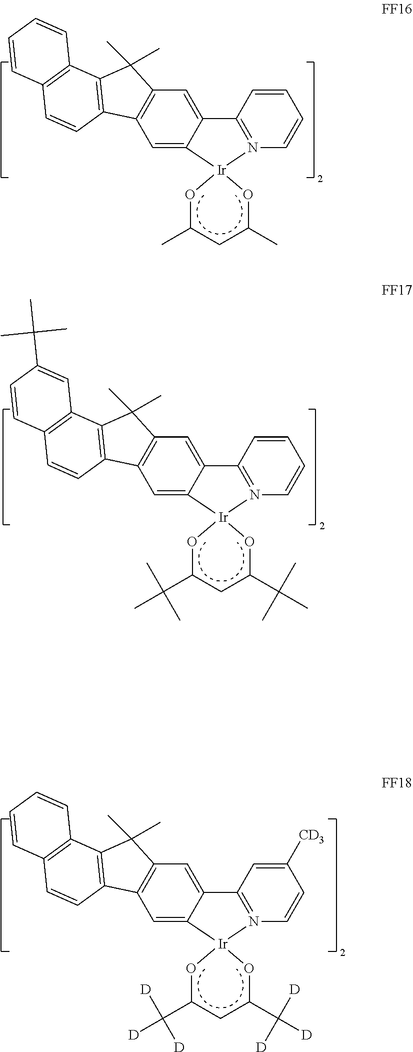

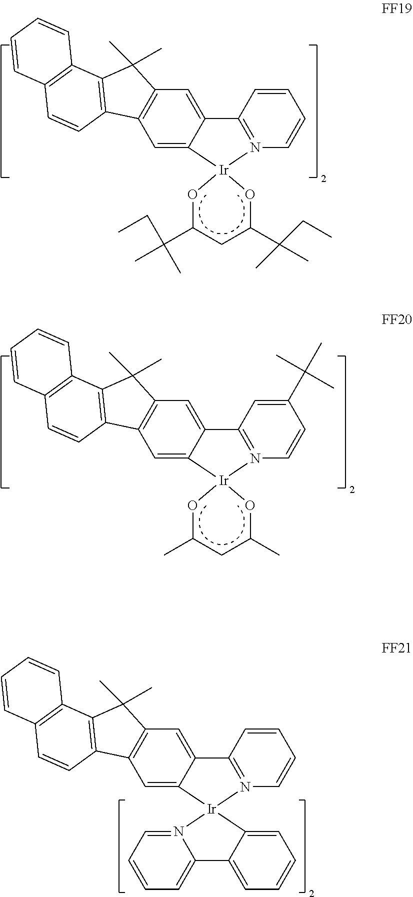

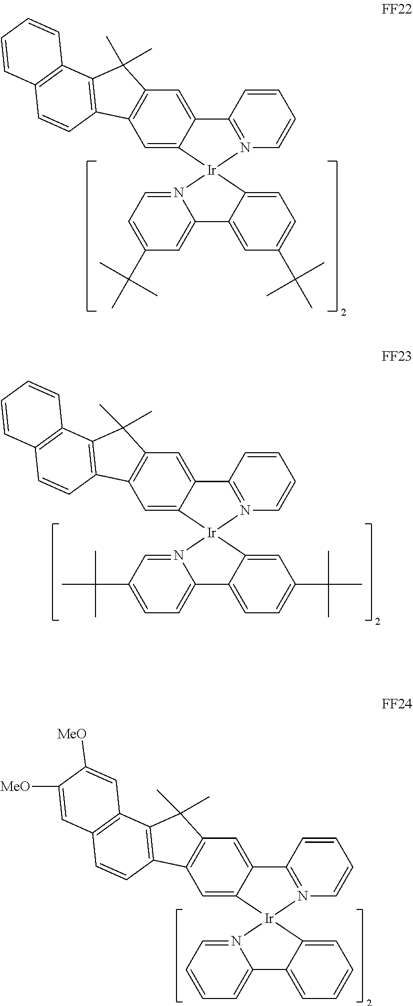

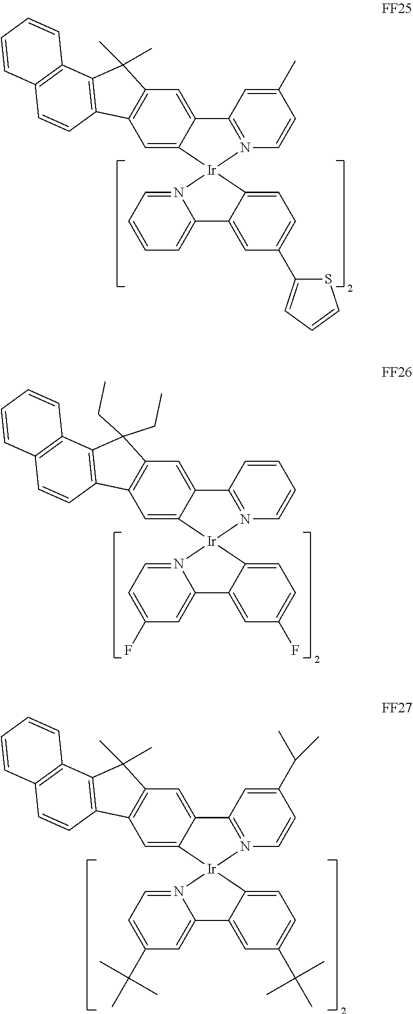

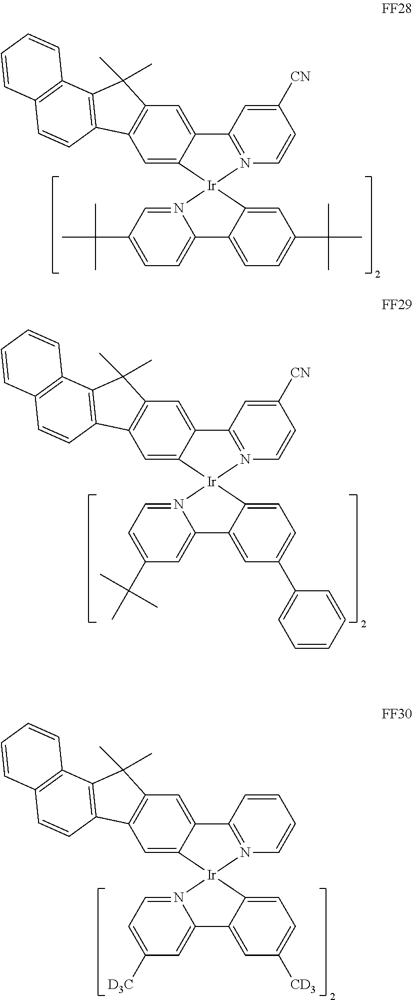

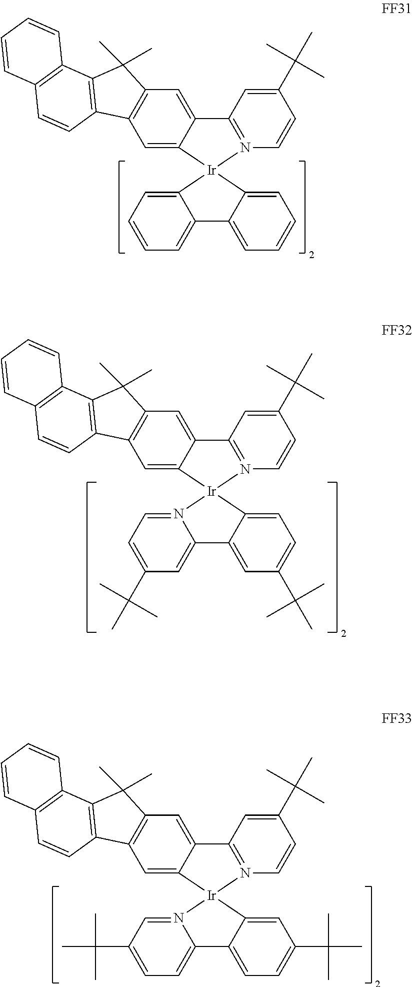

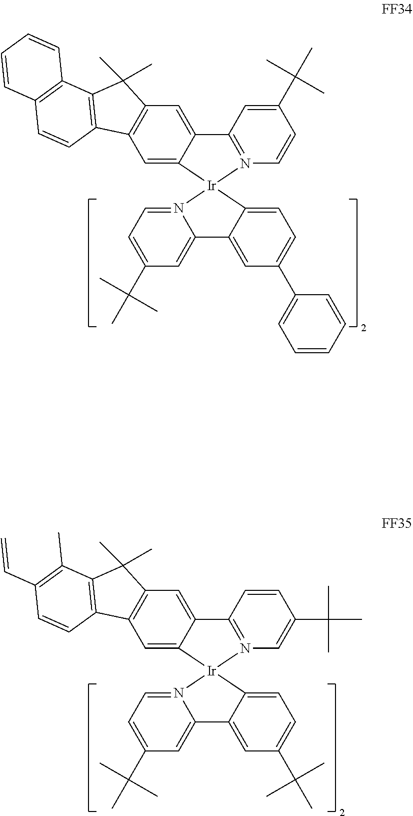

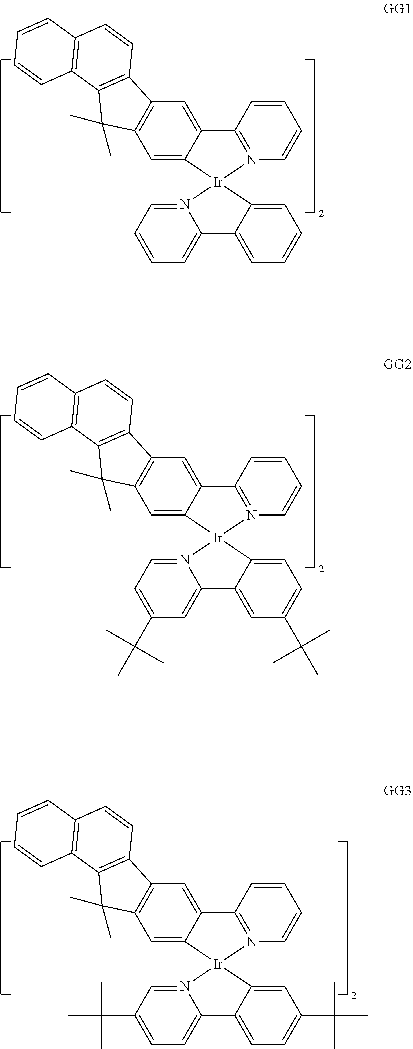

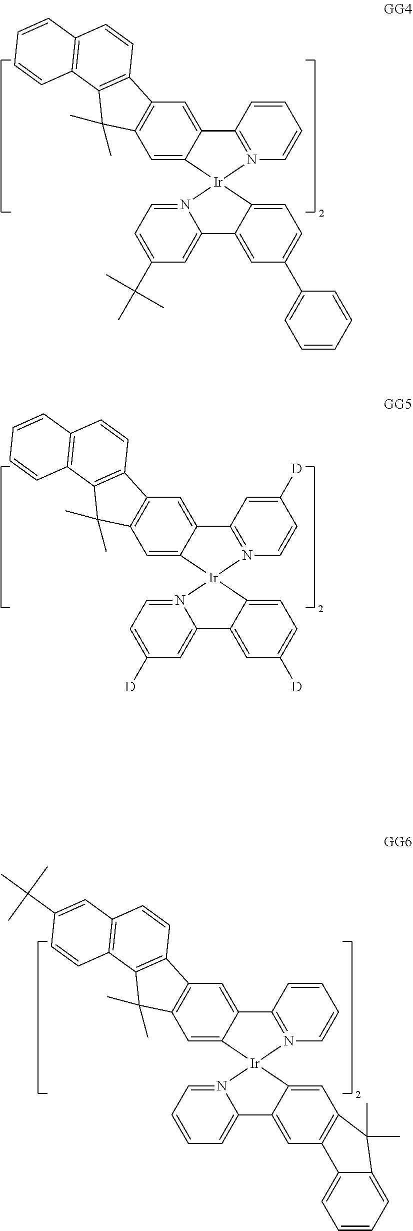

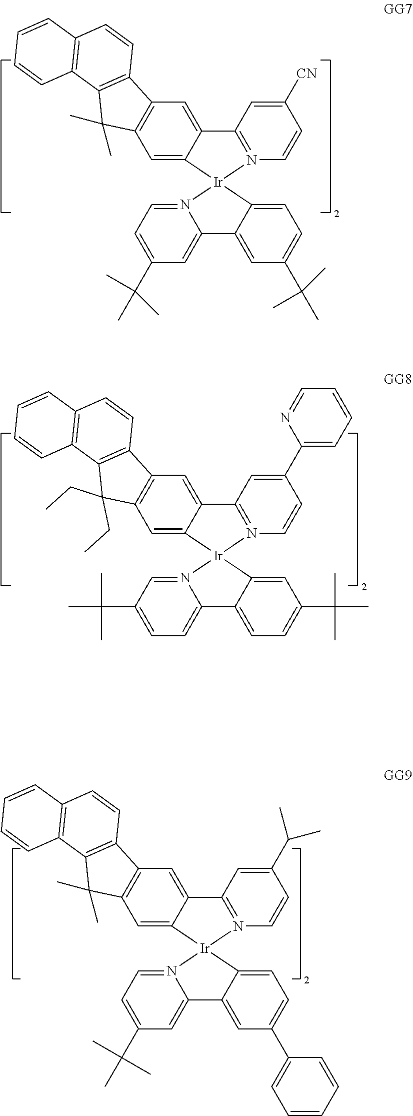

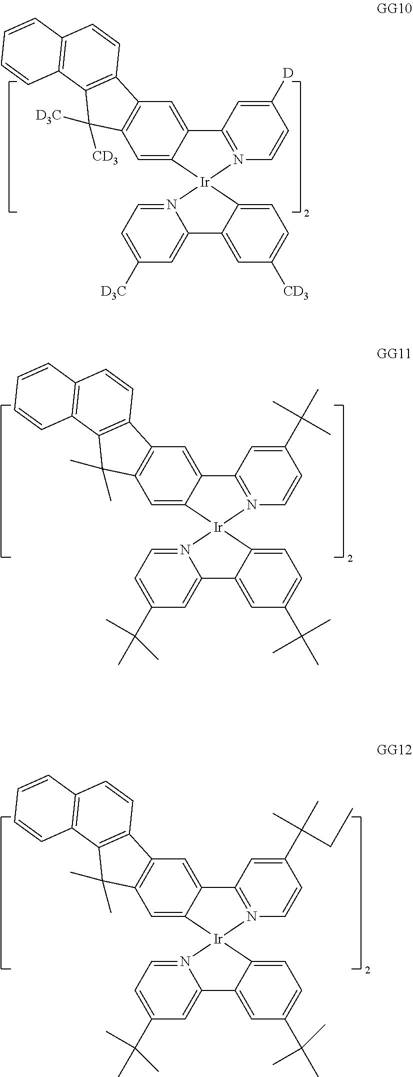

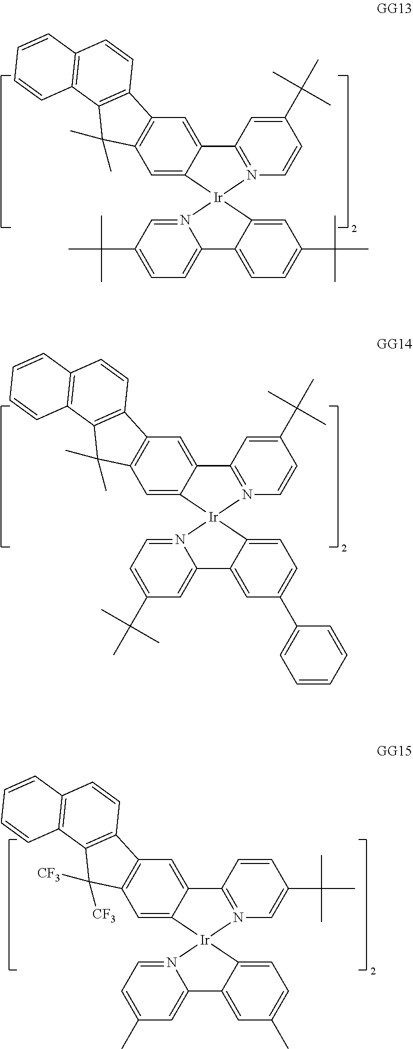

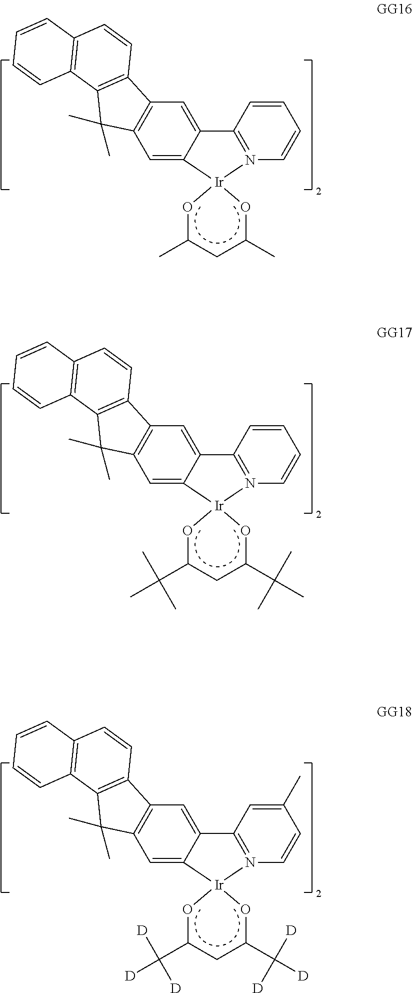

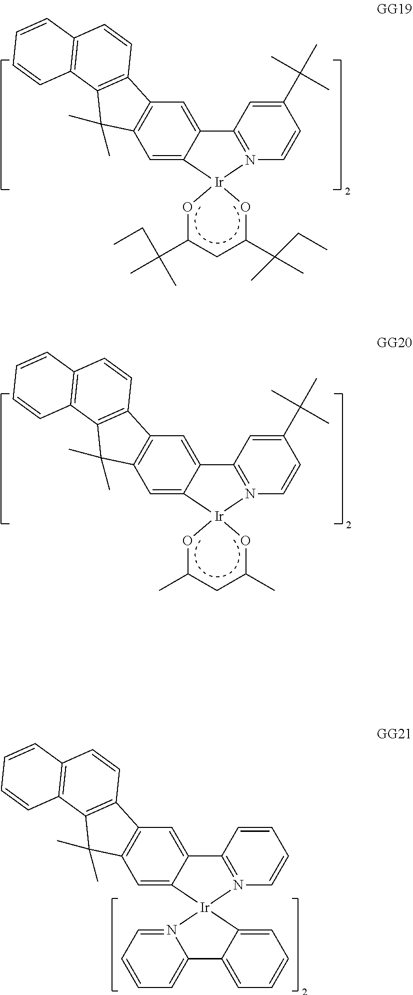

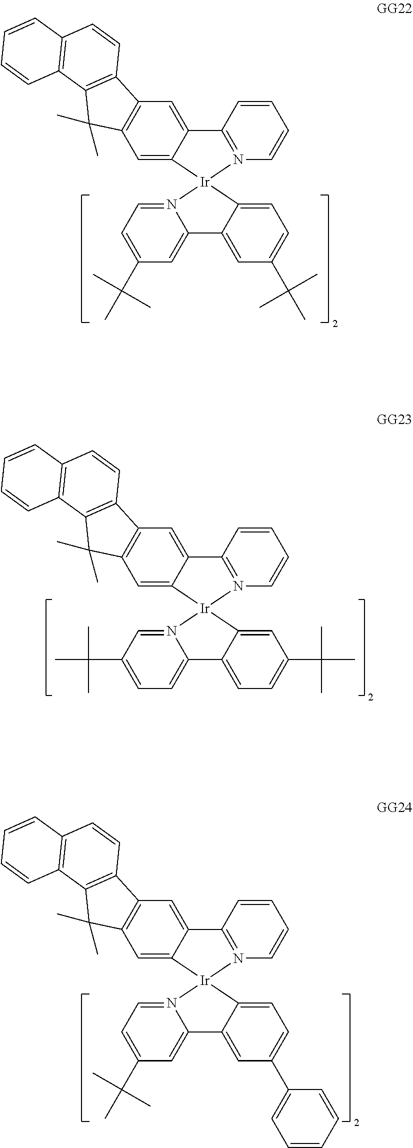

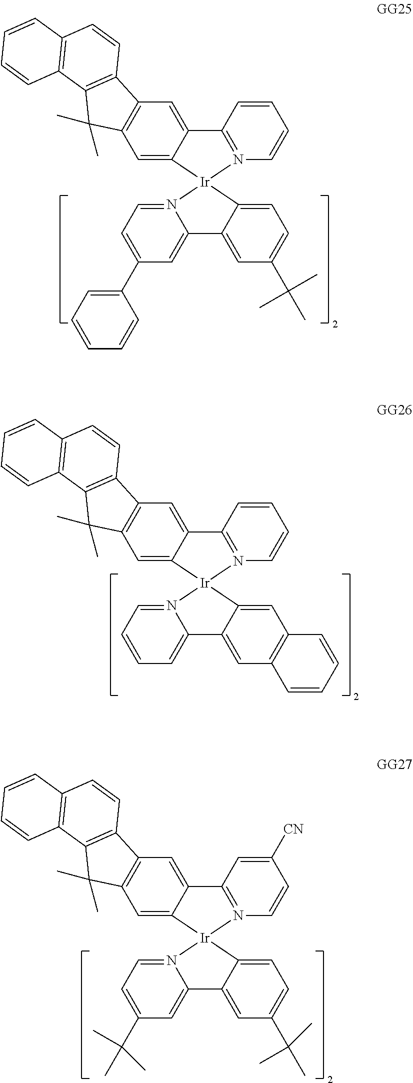

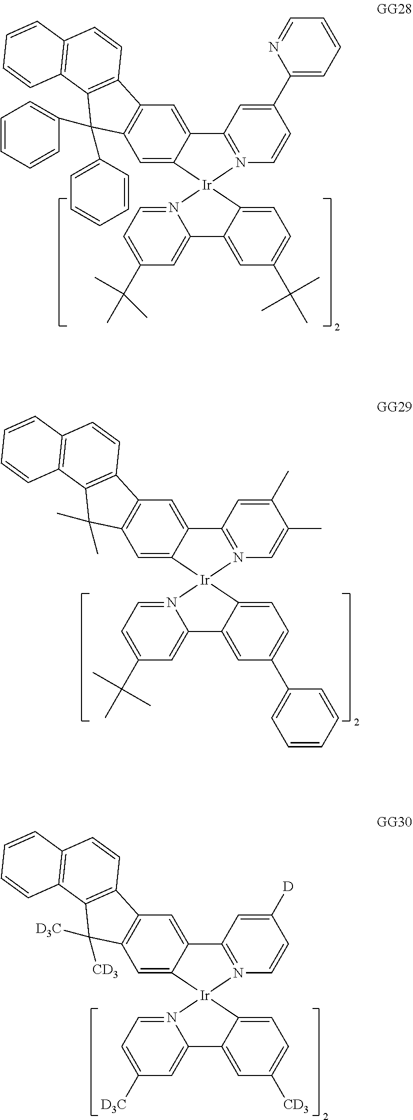

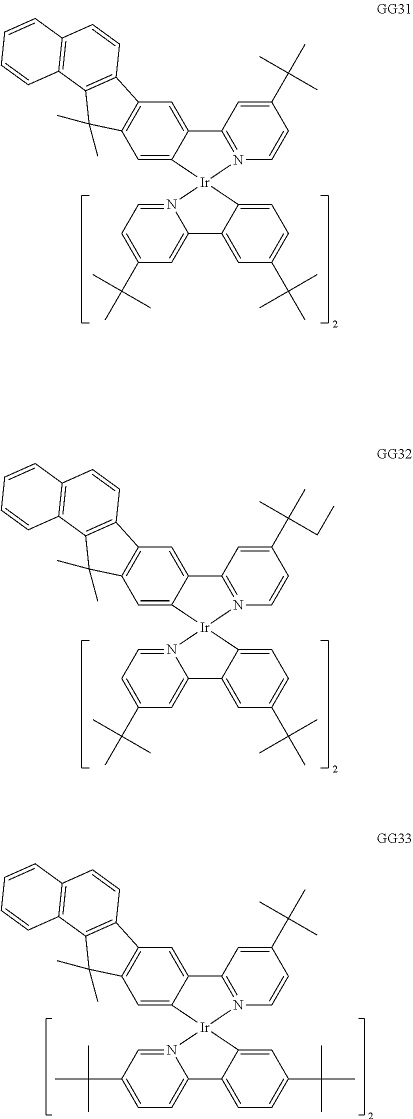

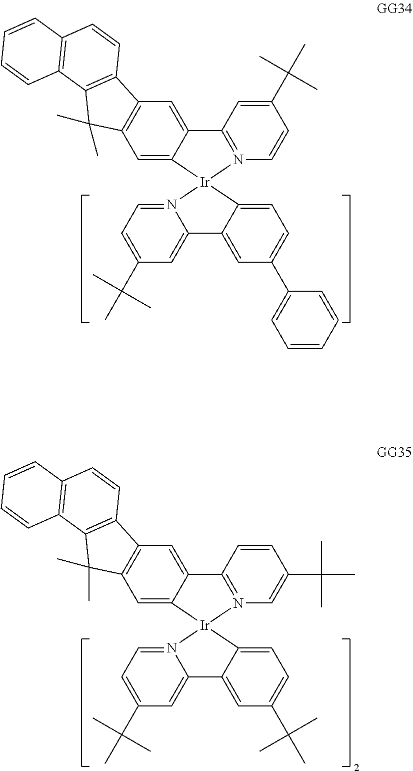

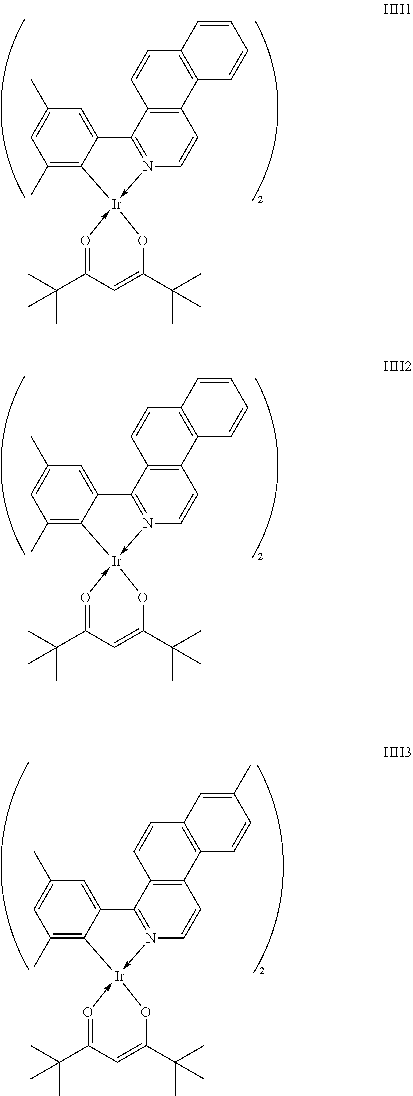

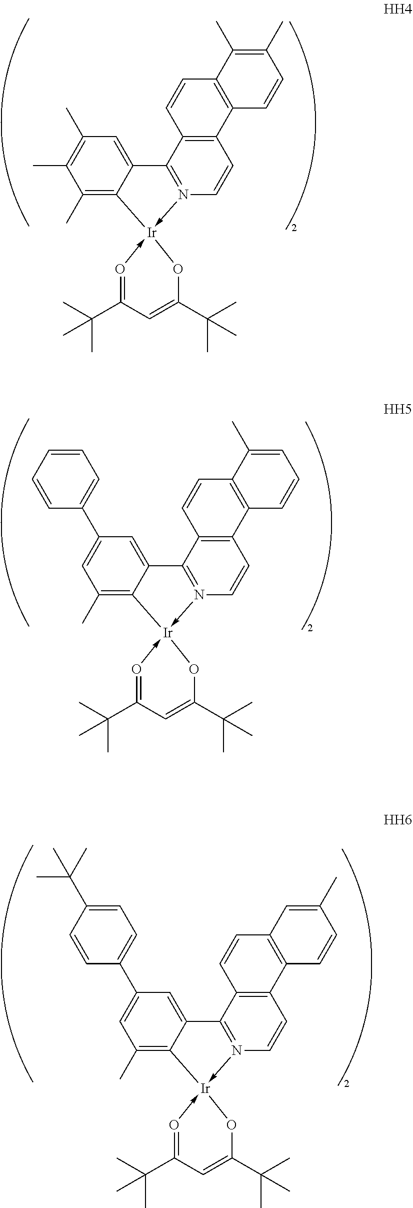

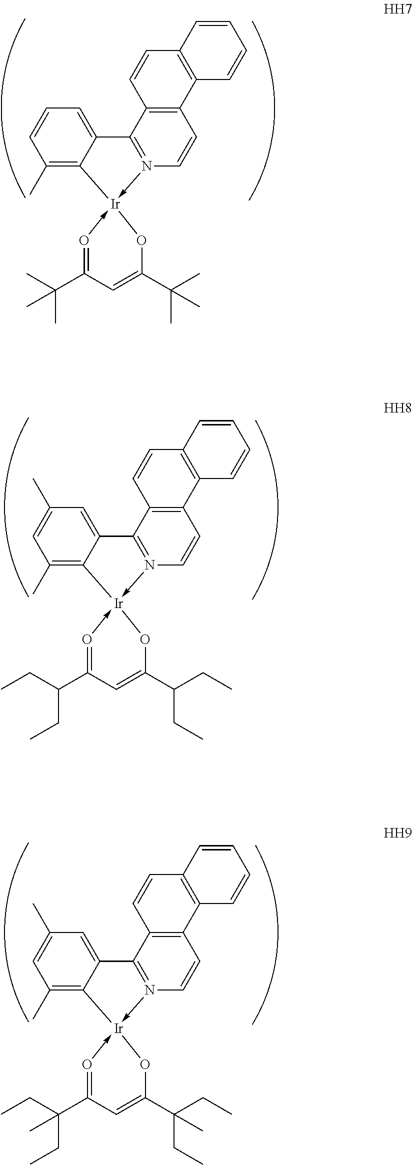

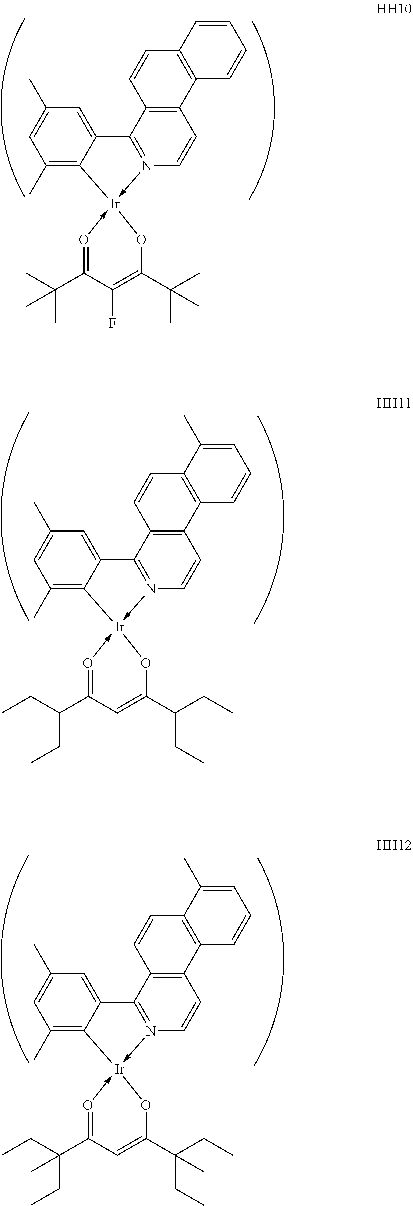

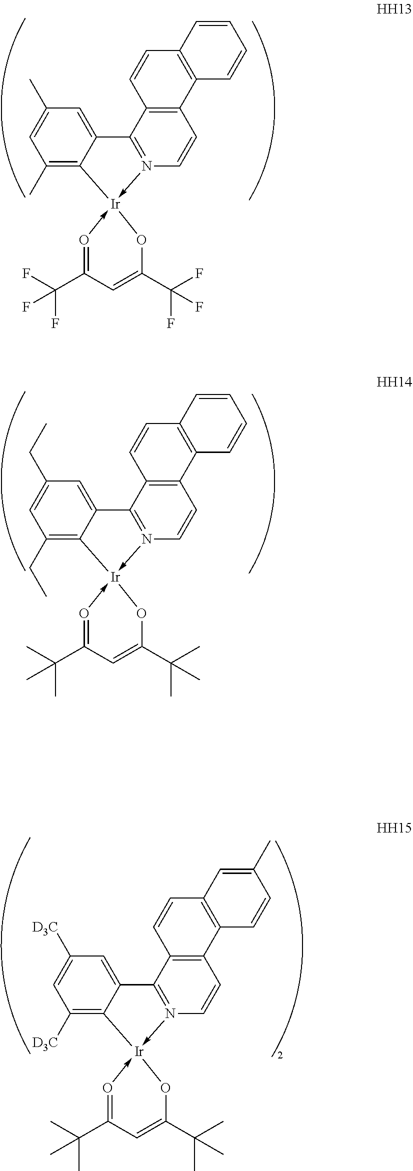

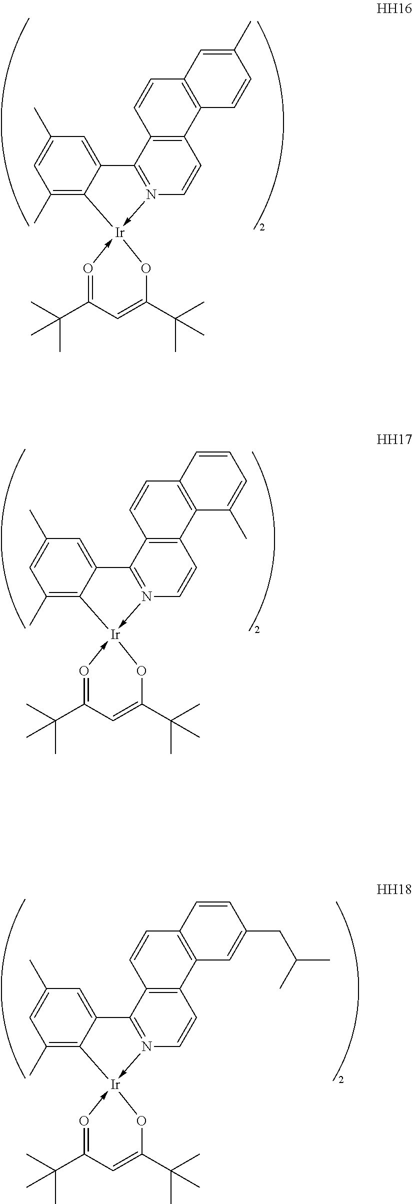

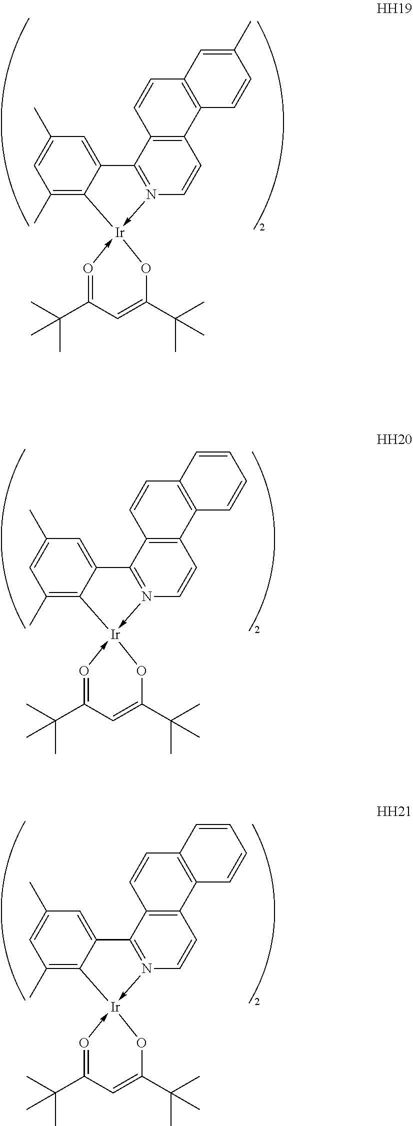

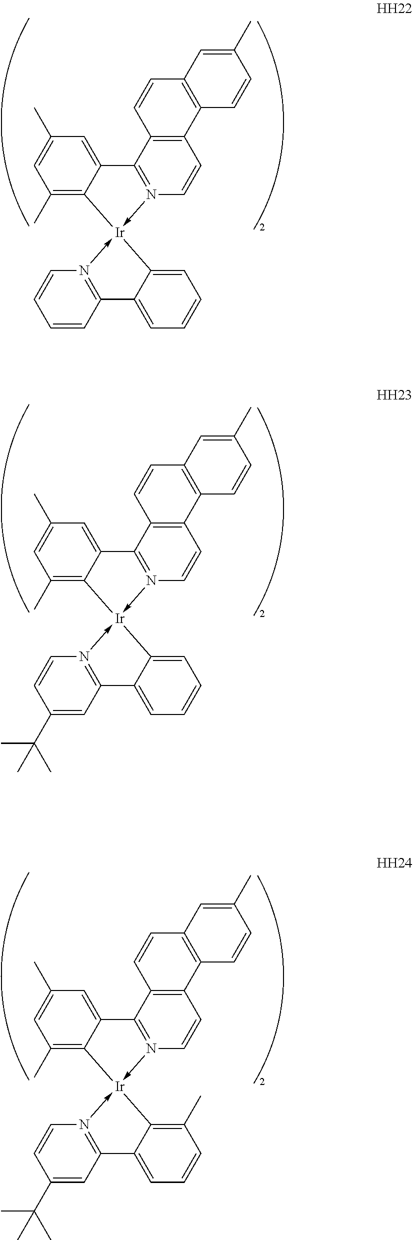

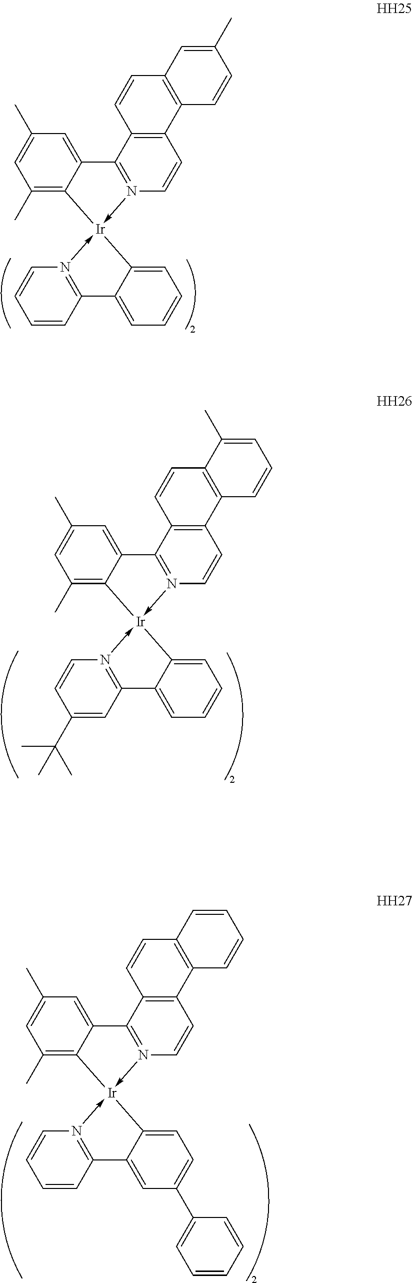

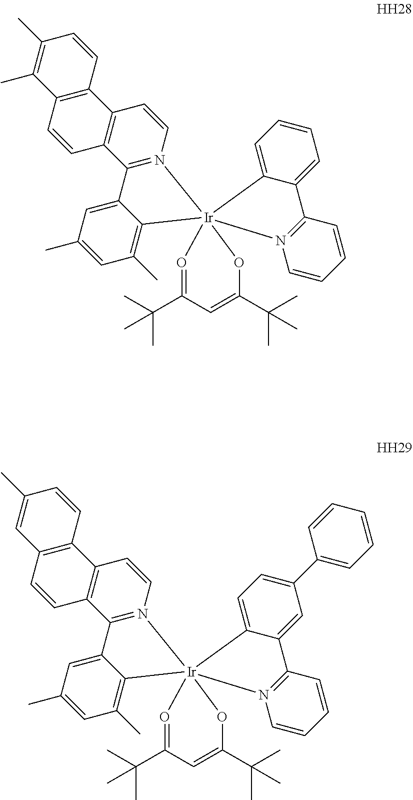

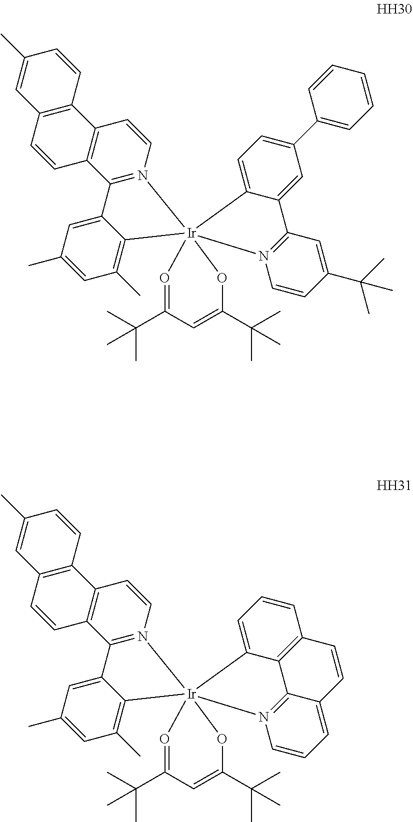

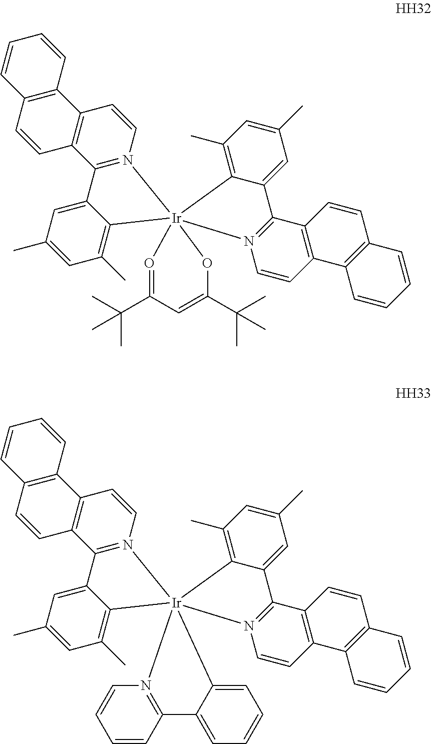

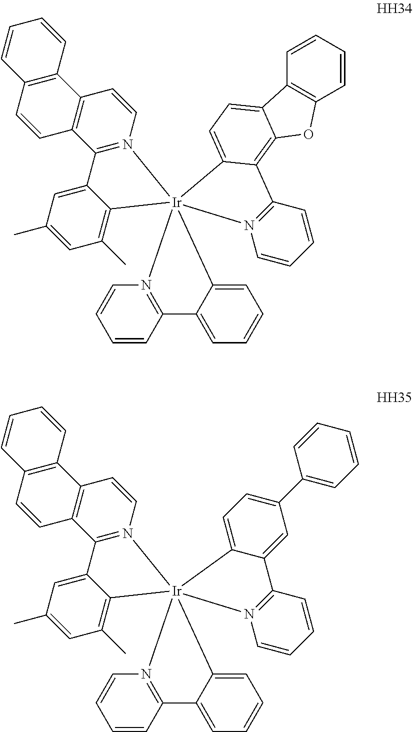

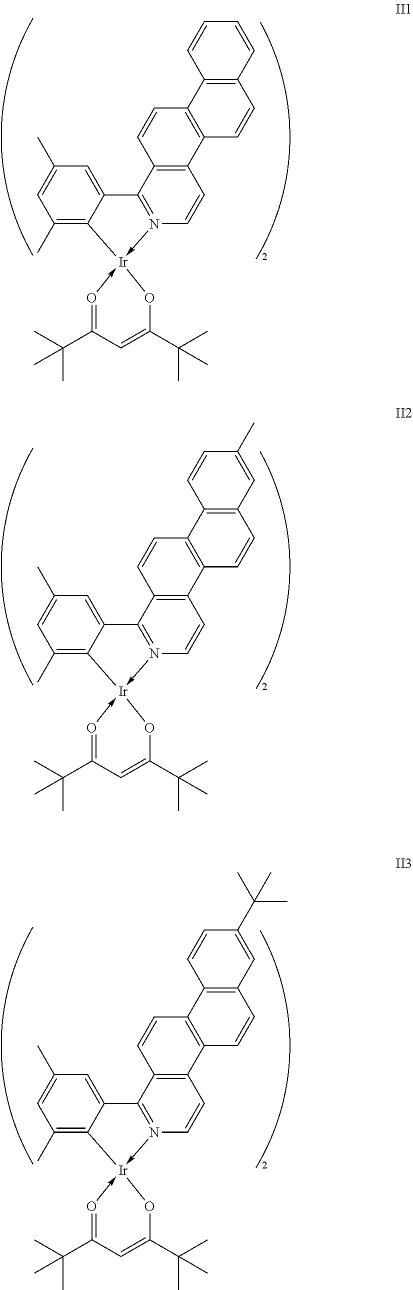

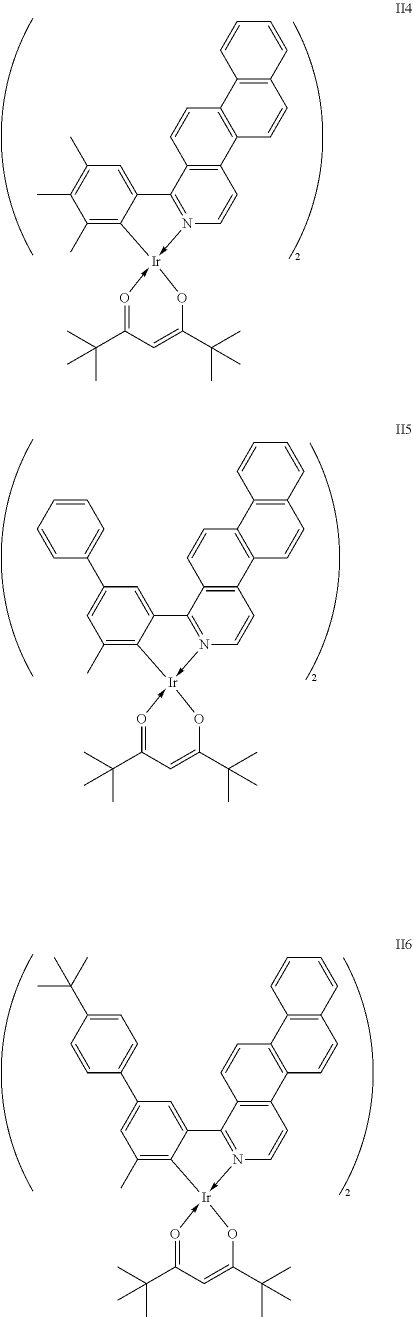

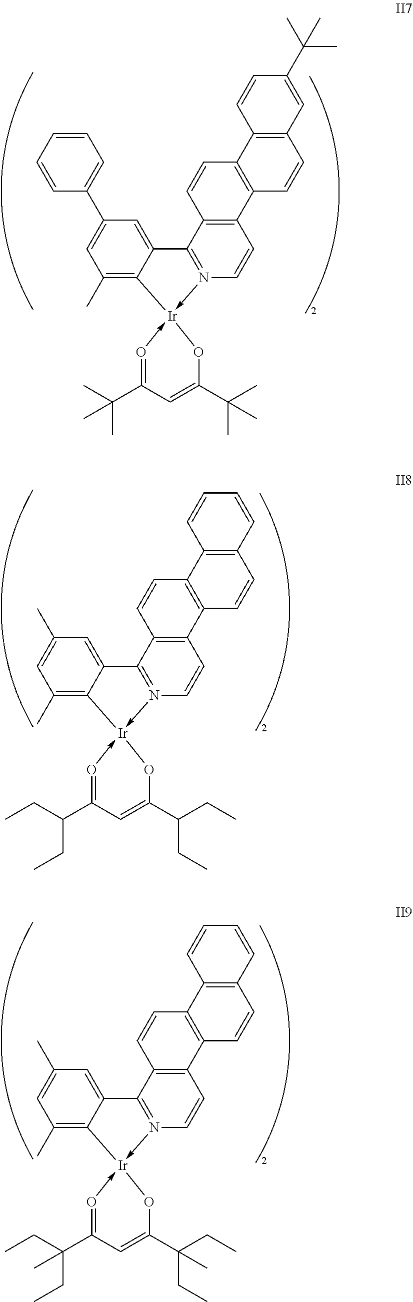

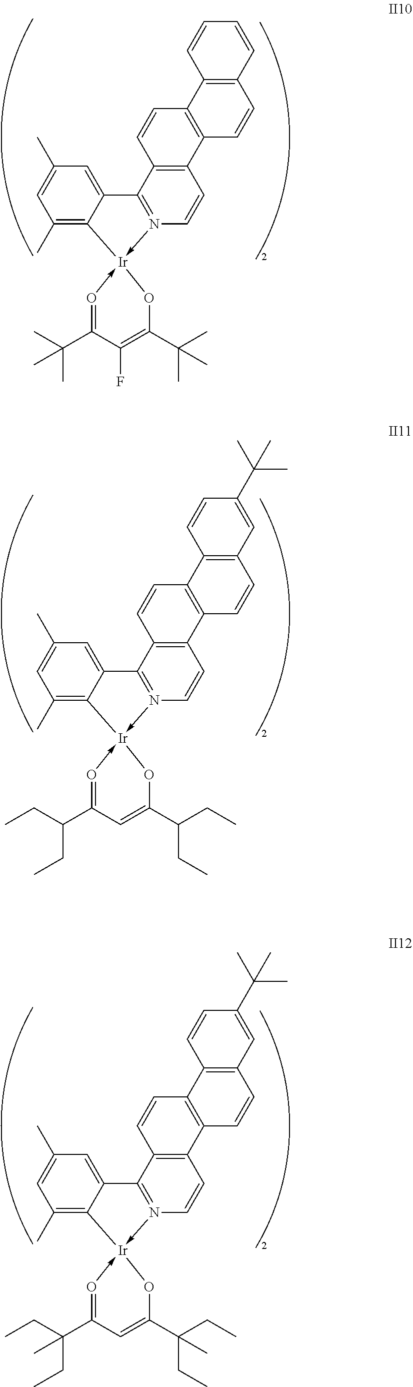

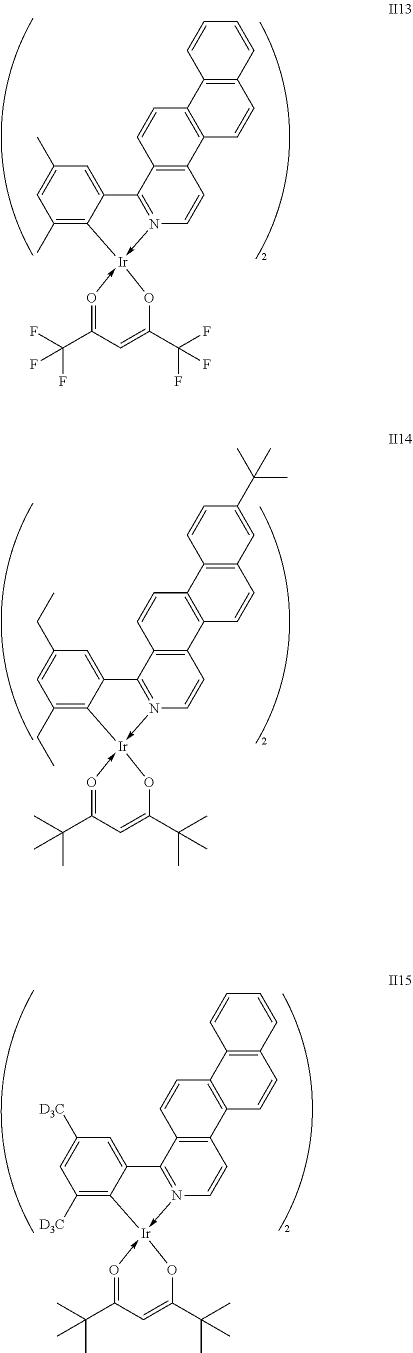

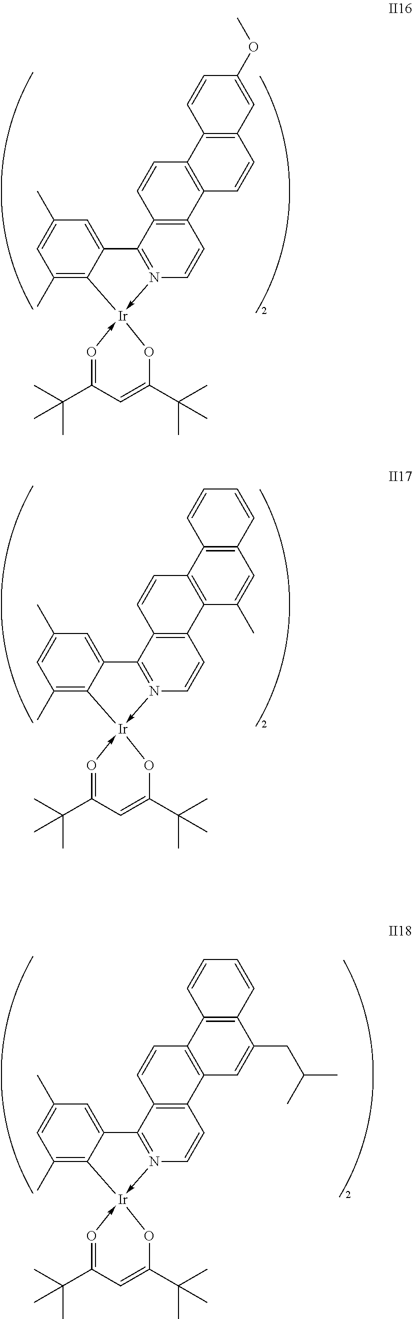

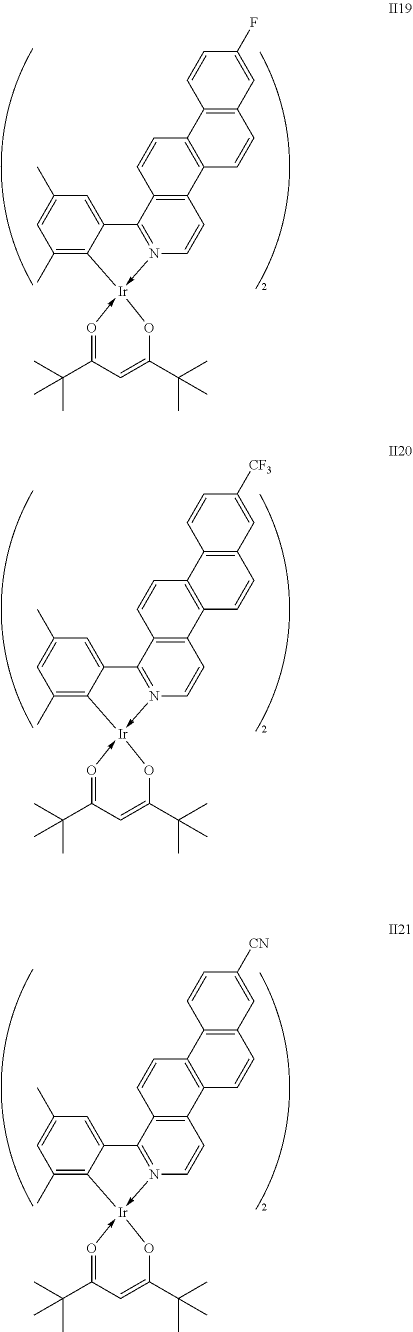

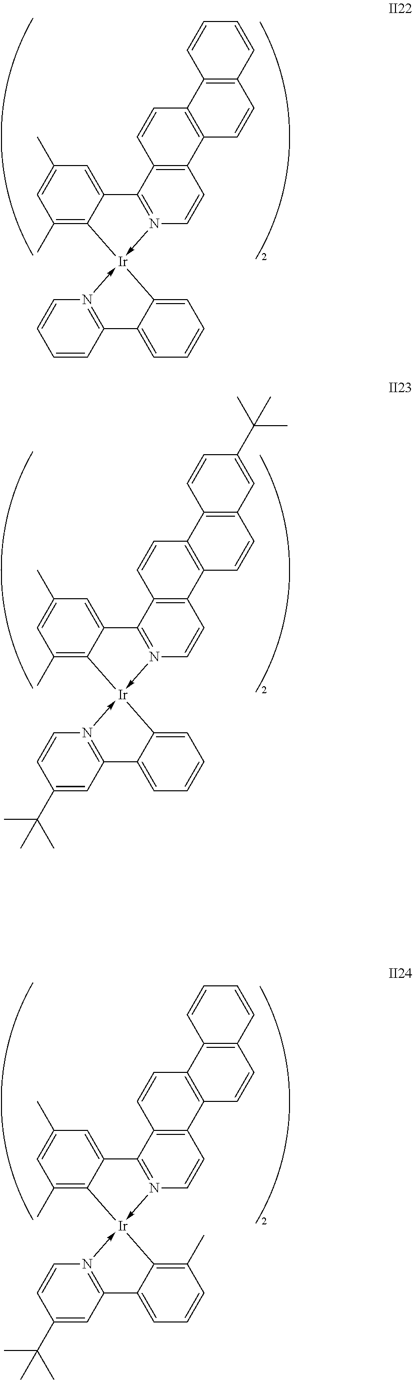

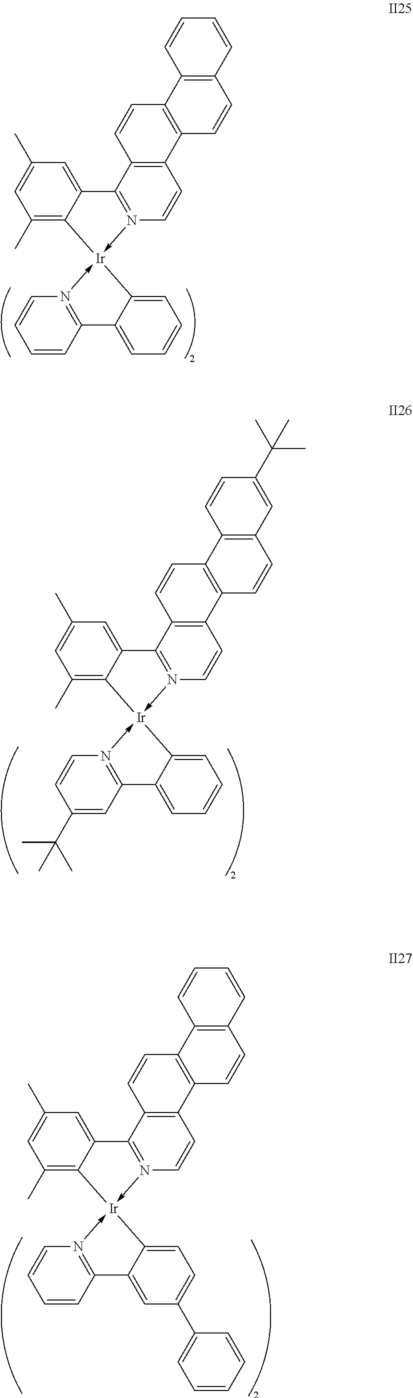

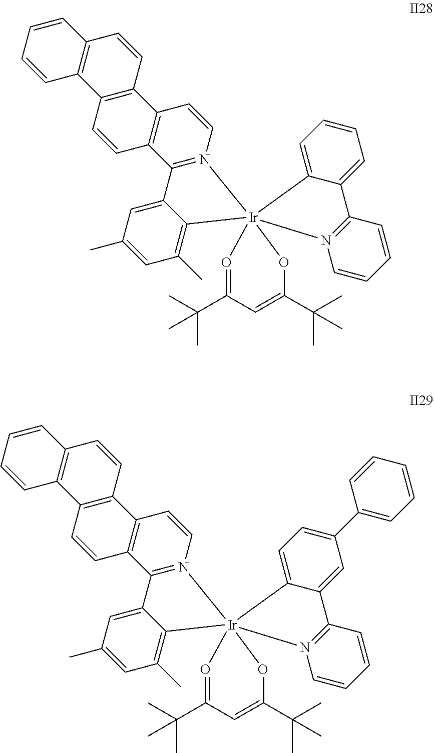

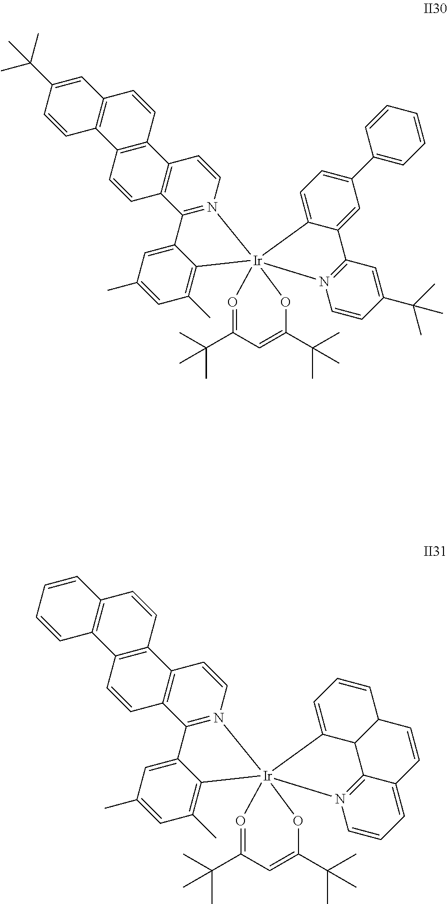

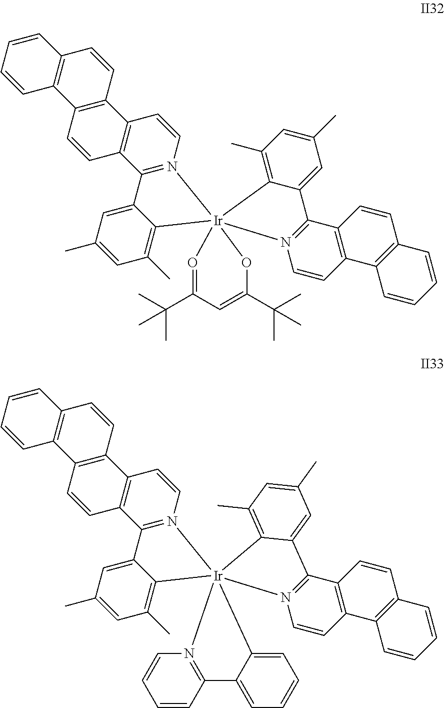

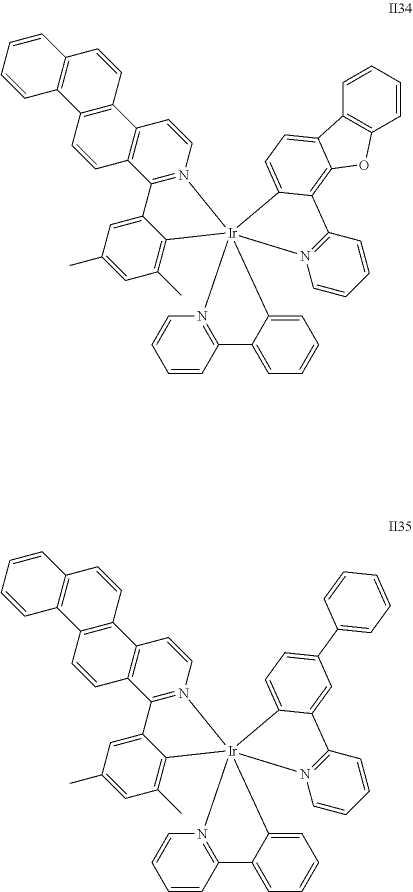

Among the above exemplified compounds each serving as the first compound, the exemplified compounds belonging to group AA to group BB are compounds each having at least a phenanthrene skeleton in the ligand of the Ir complex. The fused rings include SP2 carbons; thus, these compounds have particularly excellent stability.

Among the above exemplified compounds each serving as the first compound, the exemplified compounds belonging to group CC are compounds each having at least a triphenylene skeleton in the ligand of the Ir complex. The fused rings include SP2 carbons; thus, these compounds have particularly excellent stability.

Among the above exemplified compounds each serving as the first compound, the exemplified compounds belonging to DD group are compounds each having at least a dibenzofuran skeleton or a dibenzothiophene skeleton in the ligand of the Ir complex. These compounds contain oxygen atoms or sulfur atoms in the fused rings, so that the abundant lone pairs possessed by these atoms can enhance charge transport properties. For this reason, in particular, these compounds are compounds in which the carrier balance can be easily adjusted.

Among the above exemplified compounds each serving as the first compound, the exemplified compounds belonging to groups EE to GG are compounds each having at least a benzofluorene skeleton in the ligand of the Ir complex. Each of the compounds has a substituent at the 9-position of fluorene. In other words, each compound has the substituent in the direction perpendicular to the in-plane direction of fluorene, thereby enabling the inhibition of the overlapping of the fused rings. For this reason, in particular, these compounds have excellent sublimability.

Among the above exemplified compounds each serving as the first compound, the exemplified compounds belonging to group HH are compounds each having at least a benzoisoquinoline skeleton in the ligand of the Ir complex. These compounds can enhance the charge transport properties due to the lone pairs possessed by the nitrogen atoms and the high electronegativity. For this reason, in particular, these compounds are compounds in which the carrier balance can be easily adjusted.

Among the above exemplified compounds each serving as the first compound, the exemplified compounds belonging to group II are compounds each having at least a naphthoisoquinoline skeleton in the ligand of the Ir complex. Each of the compounds can enhance the charge transport properties due to the lone pairs possessed by the nitrogen atoms and the high electronegativity. For this reason, in particular, these compounds are compounds in which the carrier balance can be easily adjusted.

### (3) Details of Organic Light-Emitting Device

The organic light-emitting device according to an embodiment will be described below. The organic light-emitting device according to an embodiment includes at least a first electrode, a second electrode, and an organic compound layer disposed between these electrodes.

One of the first electrode and the second electrode is an anode, and the other is a cathode. In the organic light-emitting device according to the present embodiment, the organic compound layer may be formed of a single layer or a laminate including multiple layers, as long as it includes a light-emitting layer. When the organic compound layer is formed of a laminate including multiple layers, the organic compound layer may include, in addition to the light-emitting layer, a hole injection layer, a hole transport layer, an electron-blocking layer, a hole-exciton-blocking layer, an electron transport layer, and an electron injection layer, for example. The light-emitting layer may be formed of a single layer or a laminate including multiple layers.

In the organic light-emitting device according to the present embodiment, at least one layer in the organic compound layer contains the organic compound according to the present embodiment. Specifically, the organic compound according to the present embodiment is contained in any of the light-emitting layer, the hole injection layer, the hole transport layer, the electron-blocking layer, the hole-exciton-blocking layer, the electron transport layer, the electron injection layer, and so forth described above. The organic compound according to the present embodiment can be contained in the light-emitting layer.

In the organic light-emitting device according to the embodiment, when the organic compound according to the embodiment is contained in the light-emitting layer, the light-emitting layer may consist of only the organic compound according to the embodiment or may be composed of the organic compound according to the embodiment and another compound. When the light-emitting layer is composed of the organic compound according to the embodiment and another compound, the organic compound according to the embodiment may be used as a host or a guest in the light-emitting layer. The organic compound may be used as an assist material that can be contained in the light-emitting layer. The term “host” used here refers to a compound having the highest proportion by mass in compounds contained in the light-emitting layer. The term “guest” refers to a compound that has a lower proportion by mass than the host in the compounds contained in the light-emitting layer and that is responsible for main light emission. The term “assist material” refers to a compound that has a lower proportion by mass than the host in the compounds contained in the light-emitting layer and that assists the light emission of the guest. The assist material is also referred to as a “second host”. The guest material may also be referred to as a “first compound”, and the assist material may also be referred to as a “second compound”.

T1 of the second compound can be equal to or greater than T1 of the first compound.

When the organic compound according to the present embodiment is used as the host material of the light-emitting layer, the concentration of the host material is preferably 50% or more by weight and 99% or less by weight, more preferably 70% or more by weight and 99% or less by weight, based on the entire light-emitting layer.

The inventors have conducted various studies and have found that when the organic compound according to the present embodiment is used as a host material or an assist material for the light-emitting layer, particularly as an assist material for the light-emitting layer, a device that exhibits light output with high luminance at high luminous efficiency and has extremely high durability is provided. This light-emitting layer may be formed of a single layer or multiple layers. It is also possible to mix colors by making the emission color of the present embodiment blue and containing a light-emitting material having another emission color. The term “multiple layers” refers to a state in which a light-emitting layer and another light-emitting layer are stacked. In this case, the emission color of the organic light-emitting device is not limited to blue. More specifically, the emission color may be white or an intermediate color. In the case of white, another light-emitting layer emits light of a color other than blue, that is, red or green. A film-forming method is vapor deposition or coating. Details will be described in Examples below.

The organic compound according to the present embodiment can be used as a component material of an organic compound layer other than the light-emitting layer included in the organic light-emitting device according to the embodiment. Specifically, the organic compound may be used as a component material of the electron transport layer, the electron injection layer, the hole transport layer, the hole injection layer, the hole-blocking layer, and so forth. In this case, the emission color of the organic light-emitting device is not limited to blue. More specifically, the emission color may be white or intermediate color.

For example, a hole injection compound, a hole transport compound, a compound to be used as a host, a light-emitting compound, an electron injection compound, or an electron transport compound, which is known and has a low or high molecular weight, can be used together with the organic compound according to the present embodiment, as needed. Examples of these compounds are illustrated below.

As a hole injection-transport material, a material having a high hole mobility can be used so as to facilitate the injection of holes from the anode and to transport the injected holes to the light-emitting layer. To reduce a deterioration in film quality, such as crystallization, in the organic light-emitting device, a material having a high glass transition temperature can be used. Examples of a low- or high-molecular-weight material having the ability to inject and transport holes include triarylamine derivatives, aryl carbazole derivatives, phenylenediamine derivatives, stilbene derivatives, phthalocyanine derivatives, porphyrin derivatives, poly(vinyl carbazole), polythiophene, and other conductive polymers. Moreover, the hole injection-transport material can also be used for the electron-blocking layer. Non-limiting specific examples of a compound used as the hole injection-transport material will be illustrated below.

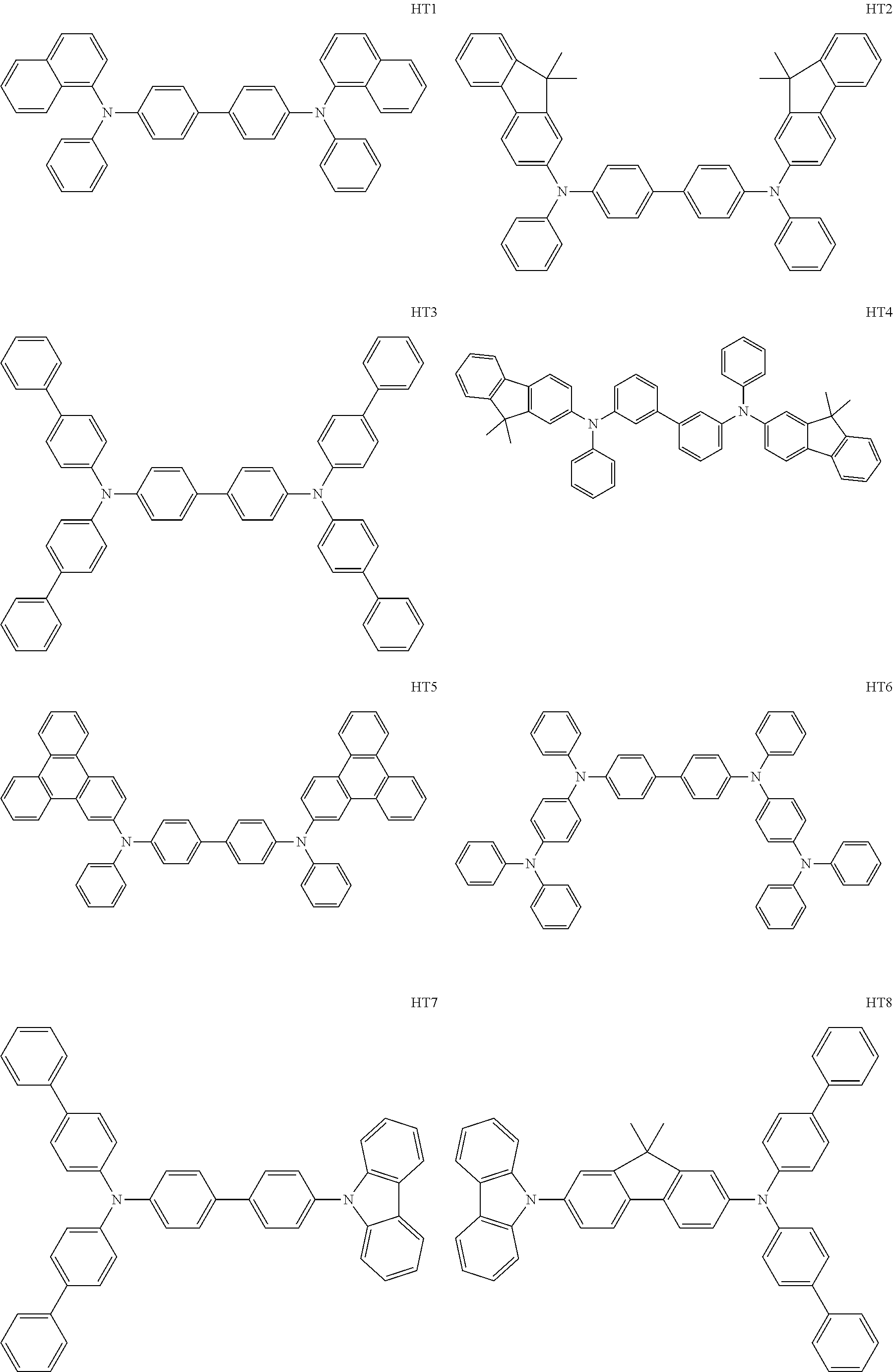

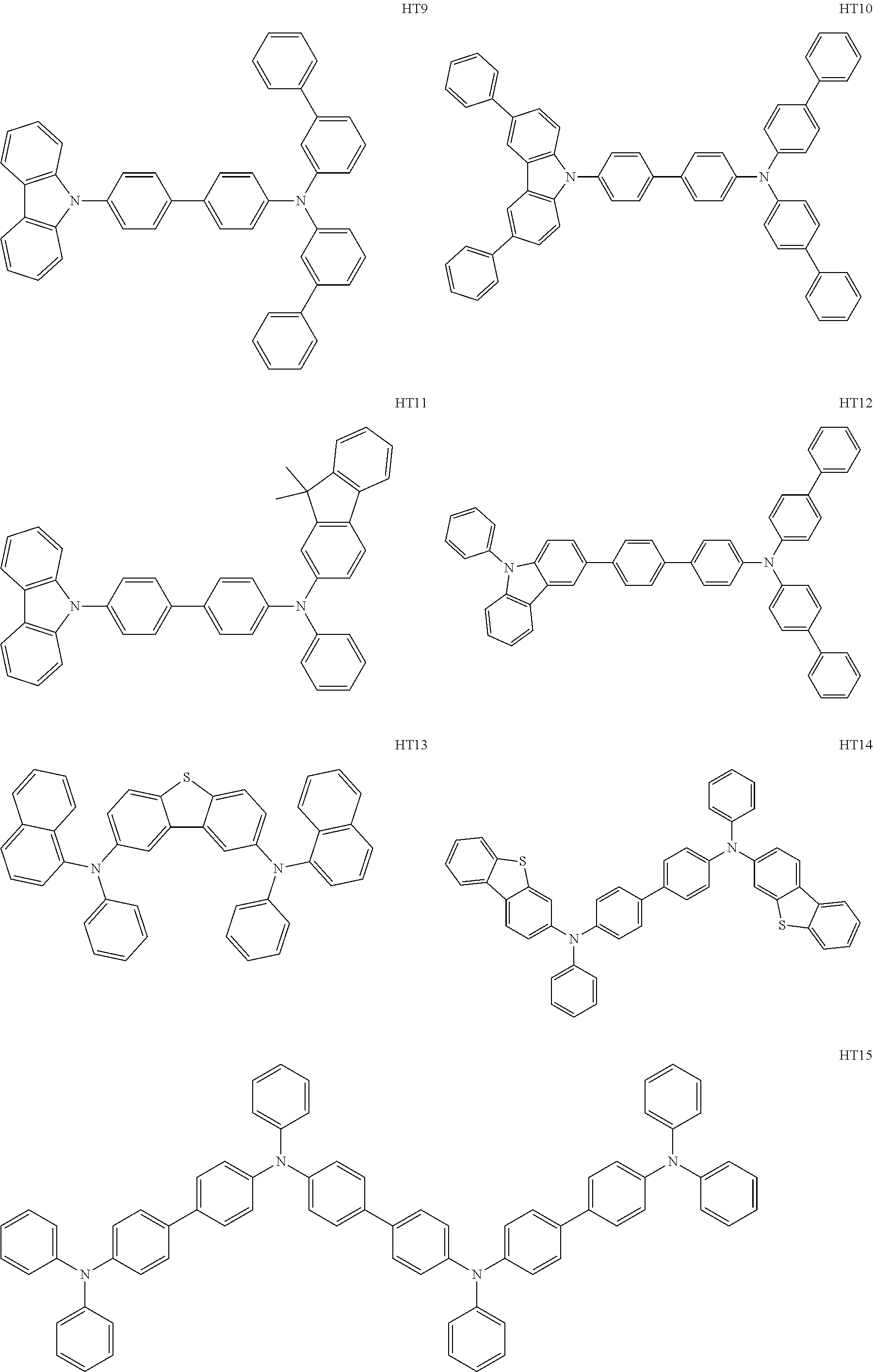

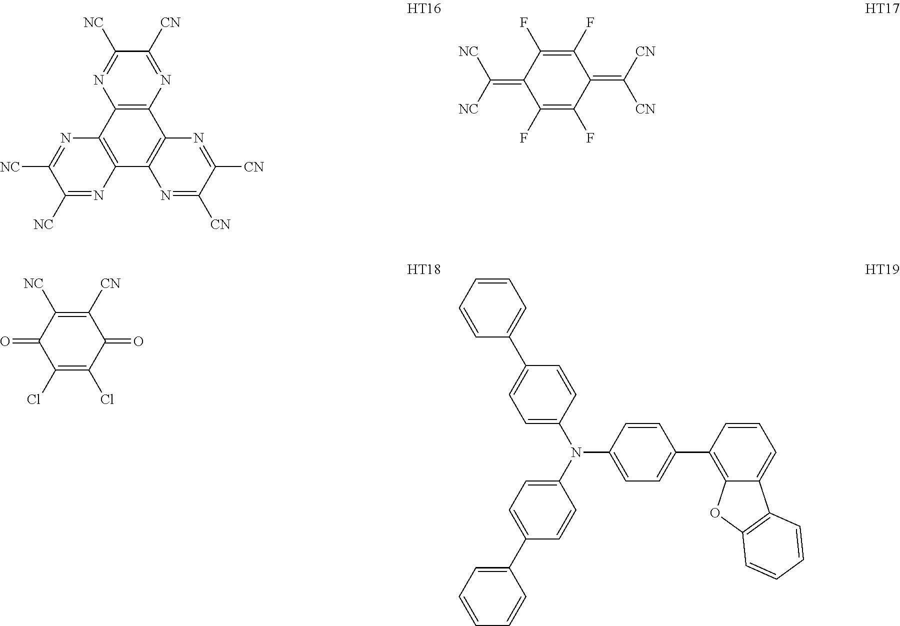 [SMILES: N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F ; N#CC1=C(C#N)C(=O)C(Cl)=C(Cl)C1=O ; c1ccc(-c2ccc(N(c3ccc(-c4ccccc4)cc3)c3ccc(-c4cccc5c4oc4ccccc45)cc3)cc2)cc1]

Among the hole transport materials illustrated above, HT16 to HT18 can be used in the layer in contact with the anode to reduce the driving voltage. HT16 is widely used in organic light-emitting devices. HT2, HT3, HT4, HT5, HT6, HT10, and HT12 may be used in an organic compound layer adjacent to HT16. Multiple materials may be used in a single organic compound layer.

Examples of a light-emitting material mainly associated with a light-emitting function include, in addition to the organic compound represented by formula (1), fused-ring compounds, such as fluorene derivatives, naphthalene derivatives, pyrene derivatives, perylene derivatives, tetracene derivatives, anthracene compounds, and rubrene, quinacridone derivatives, coumarin derivatives, stilbene derivatives, organoaluminum complexes, such as tris(8-quinolinolato)aluminum, iridium complexes, platinum complexes, rhenium complexes, copper complexes, europium complexes, ruthenium complexes, and polymer derivatives, such as poly(phenylene vinylene) derivatives, polyfluorene derivatives, and polyphenylene derivatives.

Non-limiting specific examples of a compound used as a light-emitting material are described below.

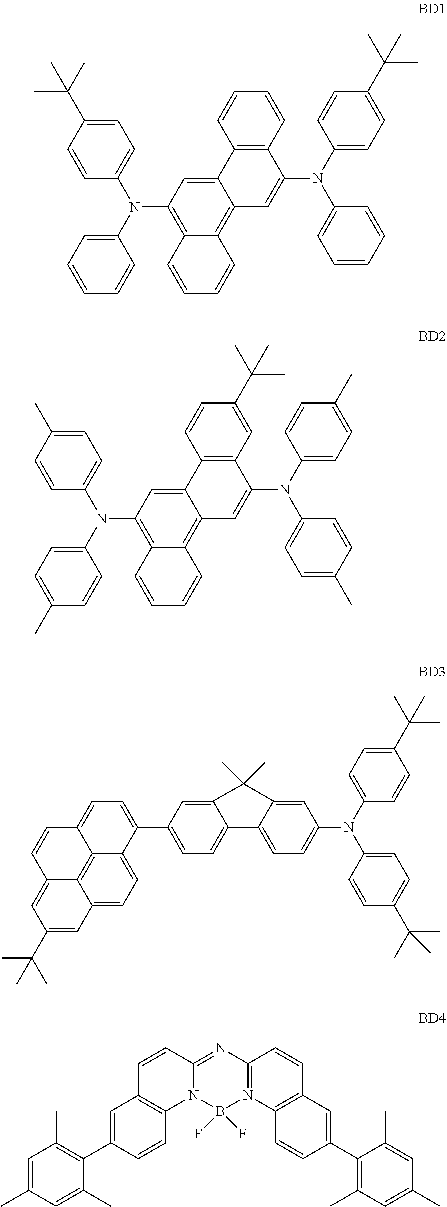

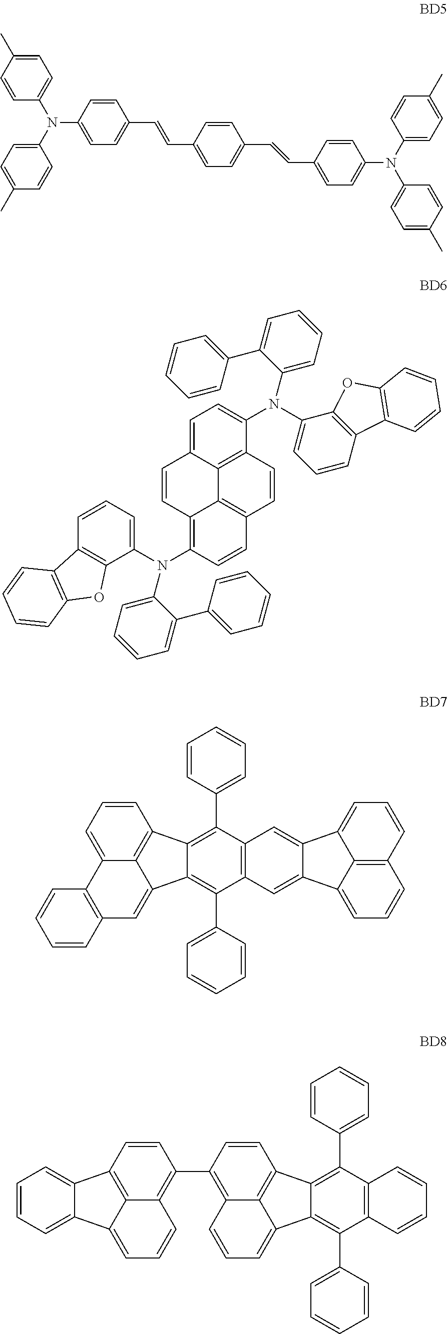

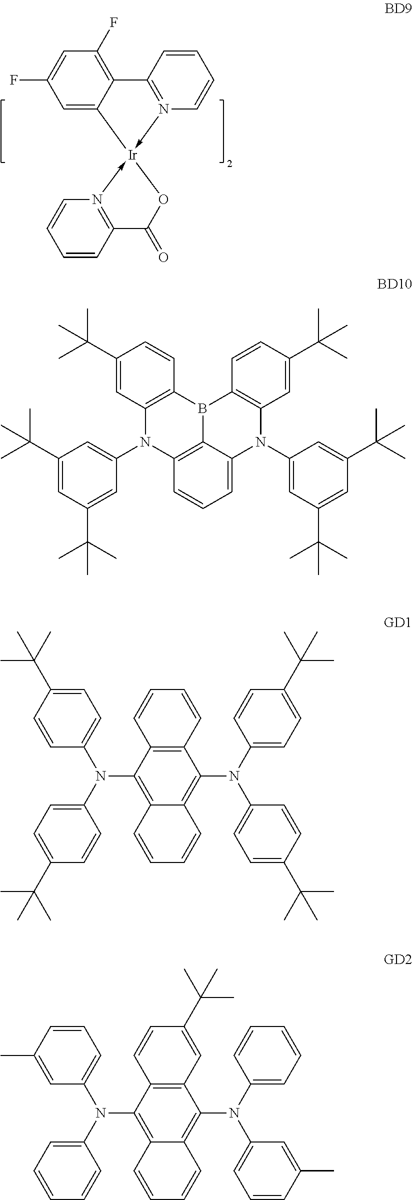

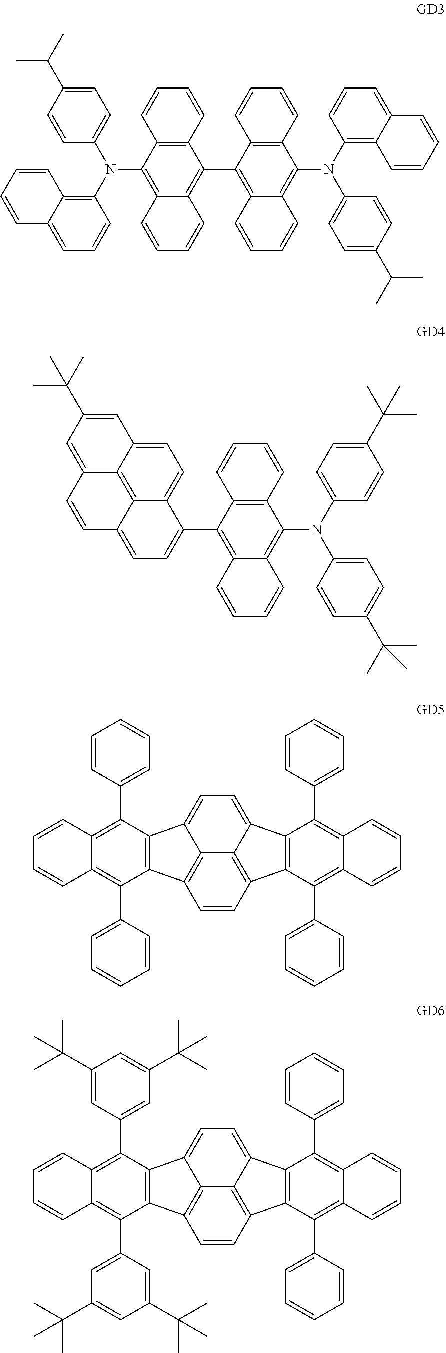

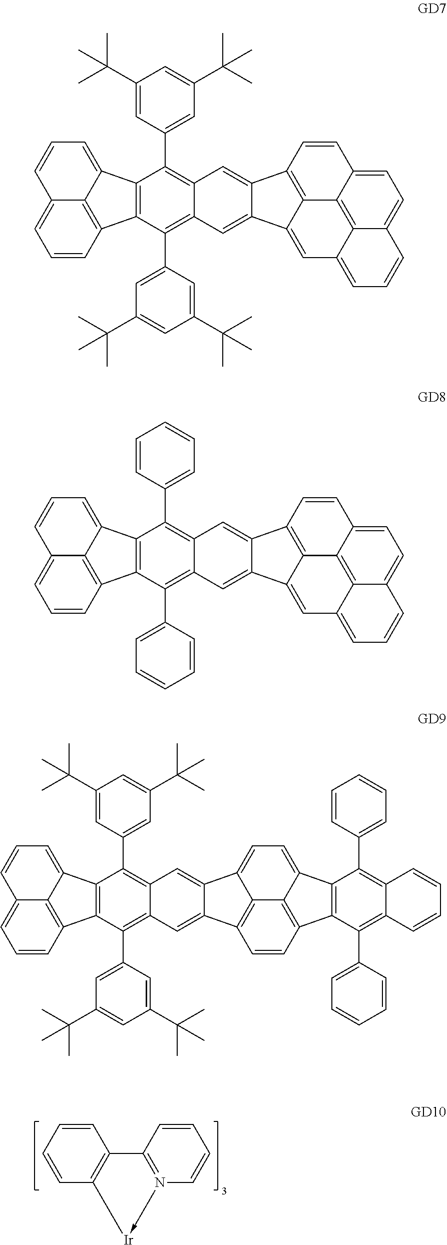

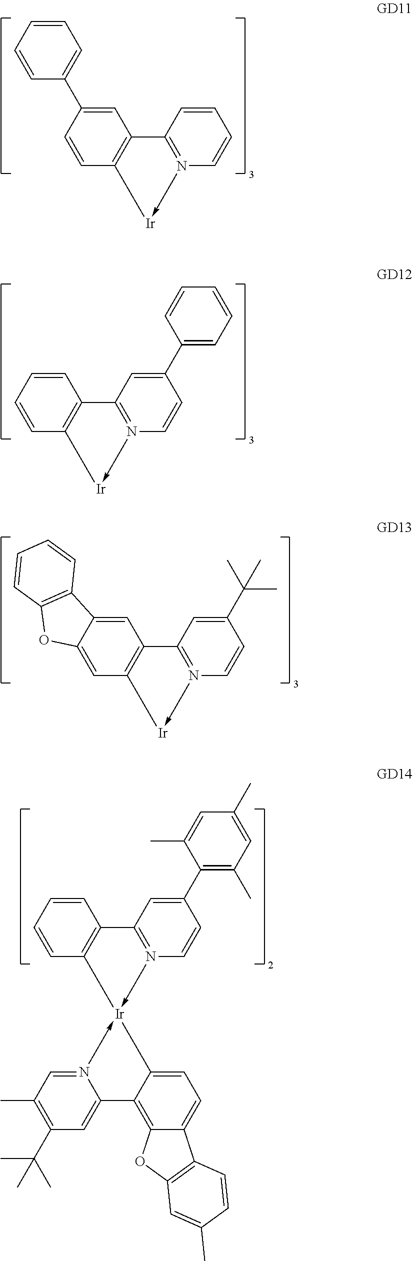

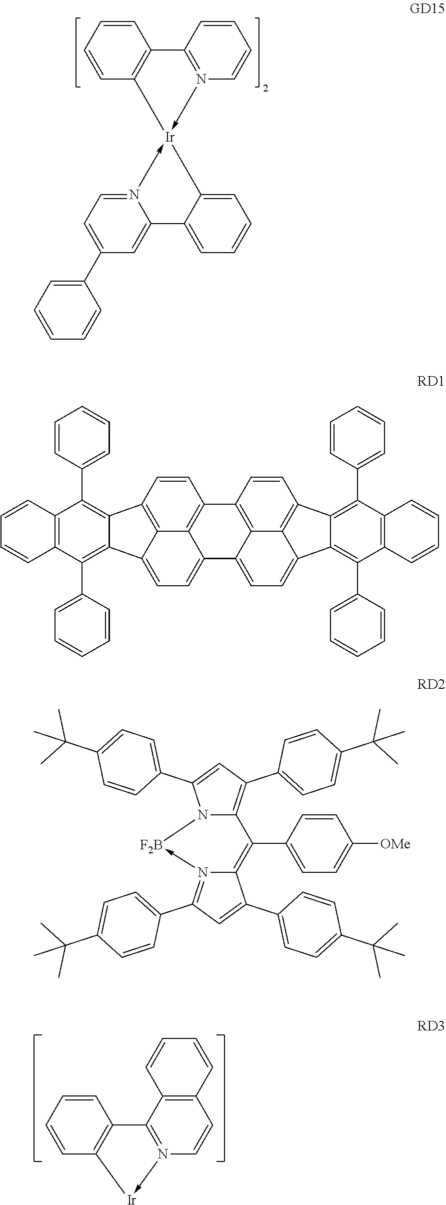

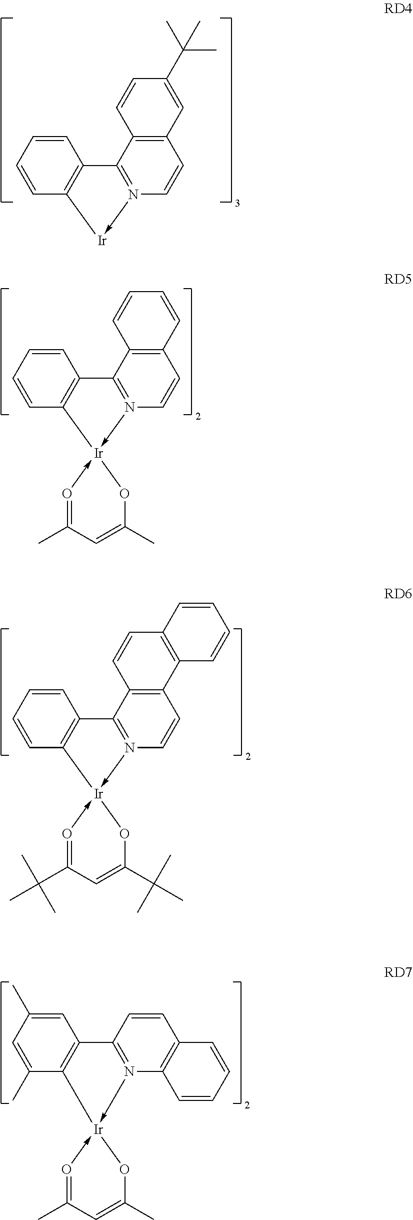

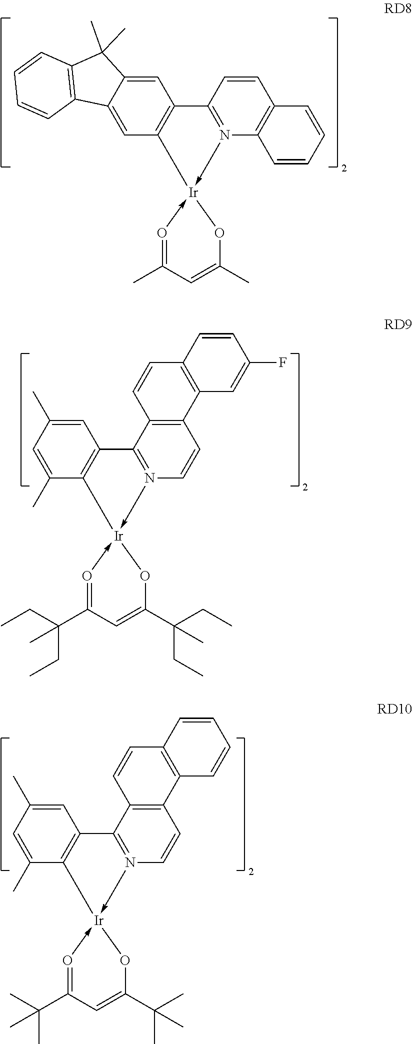

Examples of a host material or an assist material in the light-emitting layer include, in addition to the above-described exemplified compounds belonging to groups A to C, aromatic hydrocarbon compounds and derivatives thereof, carbazole derivatives, dibenzofuran derivatives, dibenzothiophene derivatives, organoaluminum complexes, such as tris(8-quinolinolato)aluminum, and organoberyllium complexes.

In particular, the assist material can contain an oxygen atom, a sulfur atom, or a nitrogen atom, or can contain a pyridine skeleton, an azine ring, a carbazole skeleton, a xanthone skeleton, or a thioxanthone skeleton. This is because these materials have high electron-donating or electron-withdrawing properties, so that the HOMO or LUMO level can be easily adjusted. T1 of the assist material can be higher than T1 of the organic compound according to an embodiment of the present disclosure.

The organic compound according to an embodiment of the present disclosure has a structure in which three or more fused rings are attached to benzene and thus has a wide band gap to some extent. Therefore, a material having the above skeleton capable of adjusting the HOMO or LUMO level can be particularly used as the assist material. A good carrier balance can be achieved when these assist materials are combined with the organic compound according to an embodiment of the present disclosure.

Non-limiting specific examples of a compound used as a host or assist material in the light-emitting layer will be illustrated below.

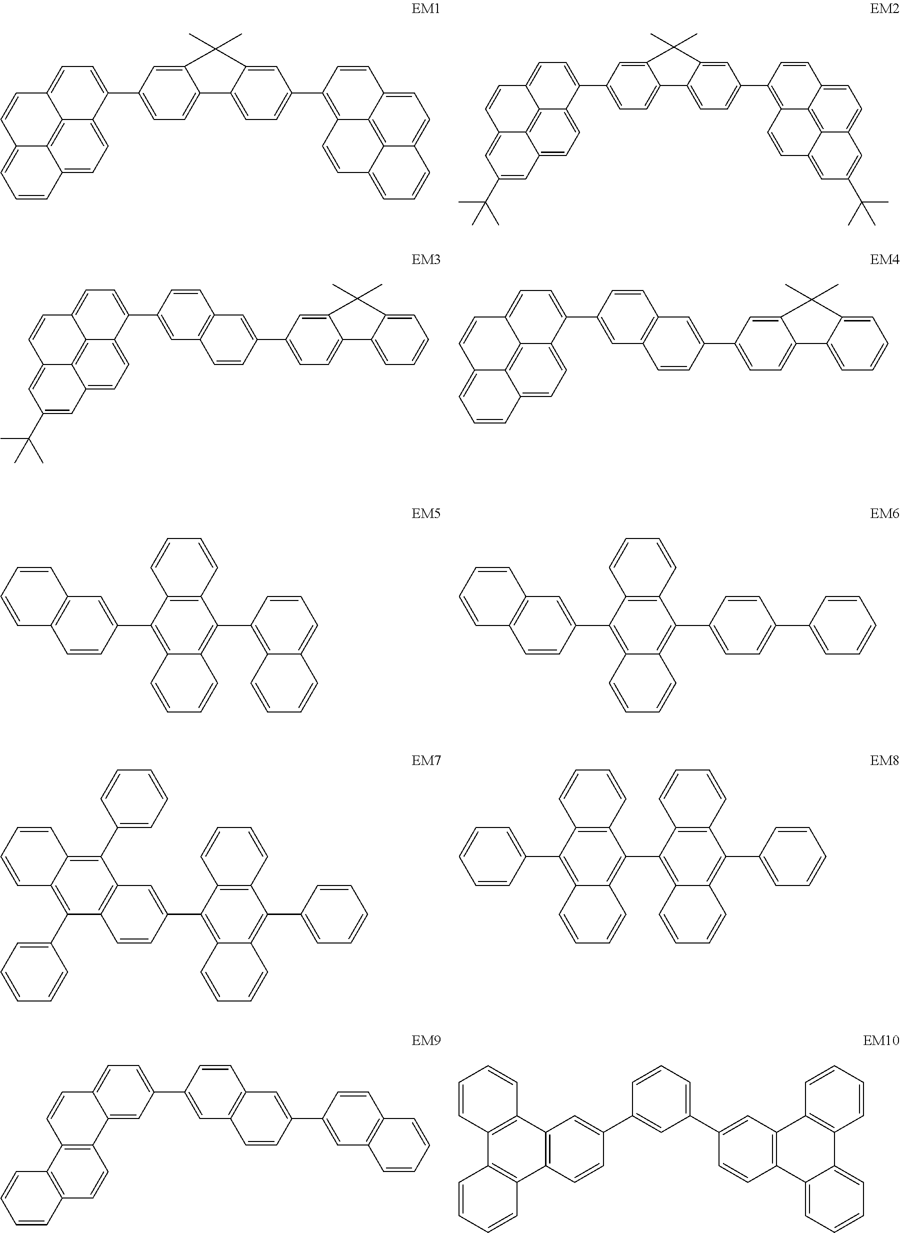

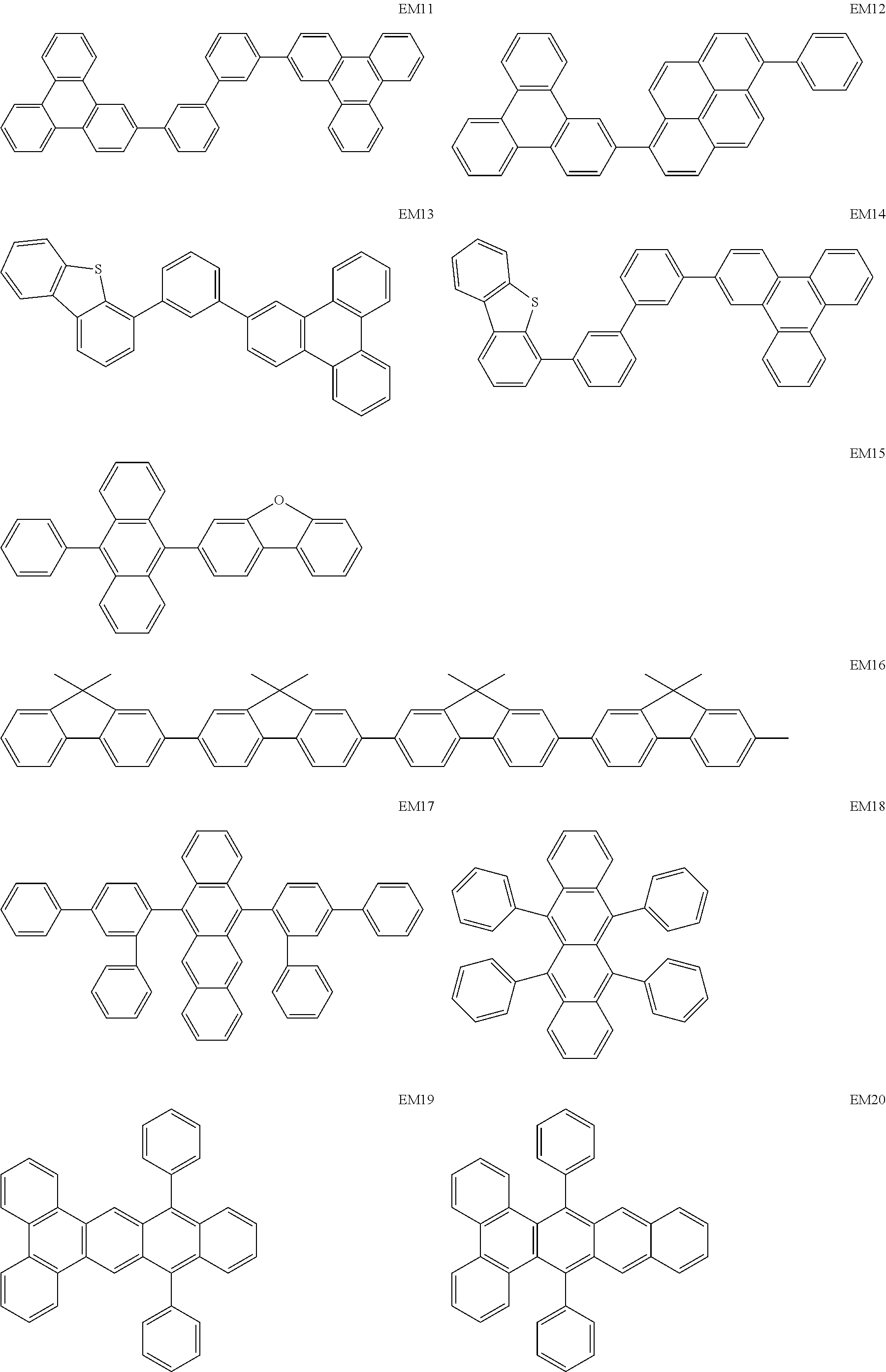

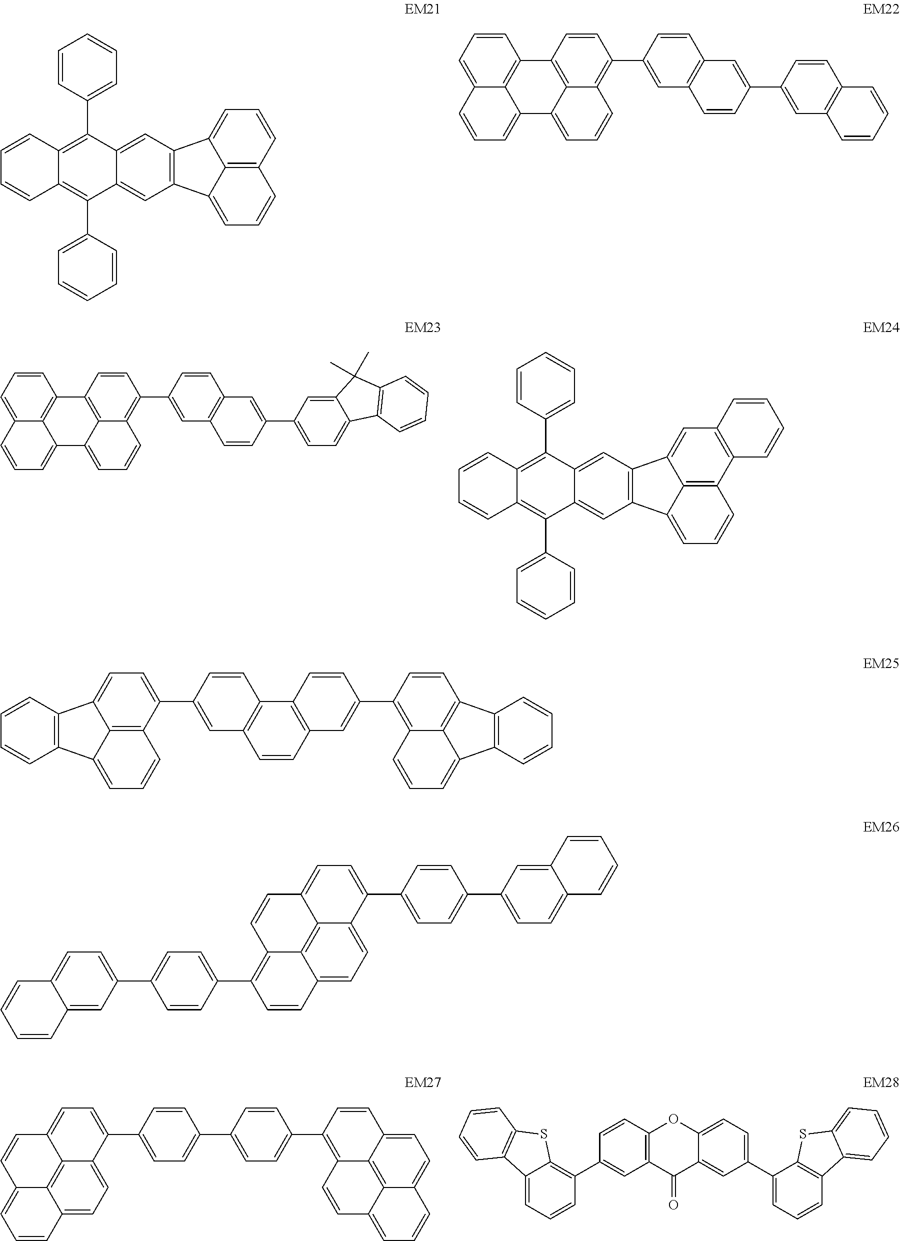

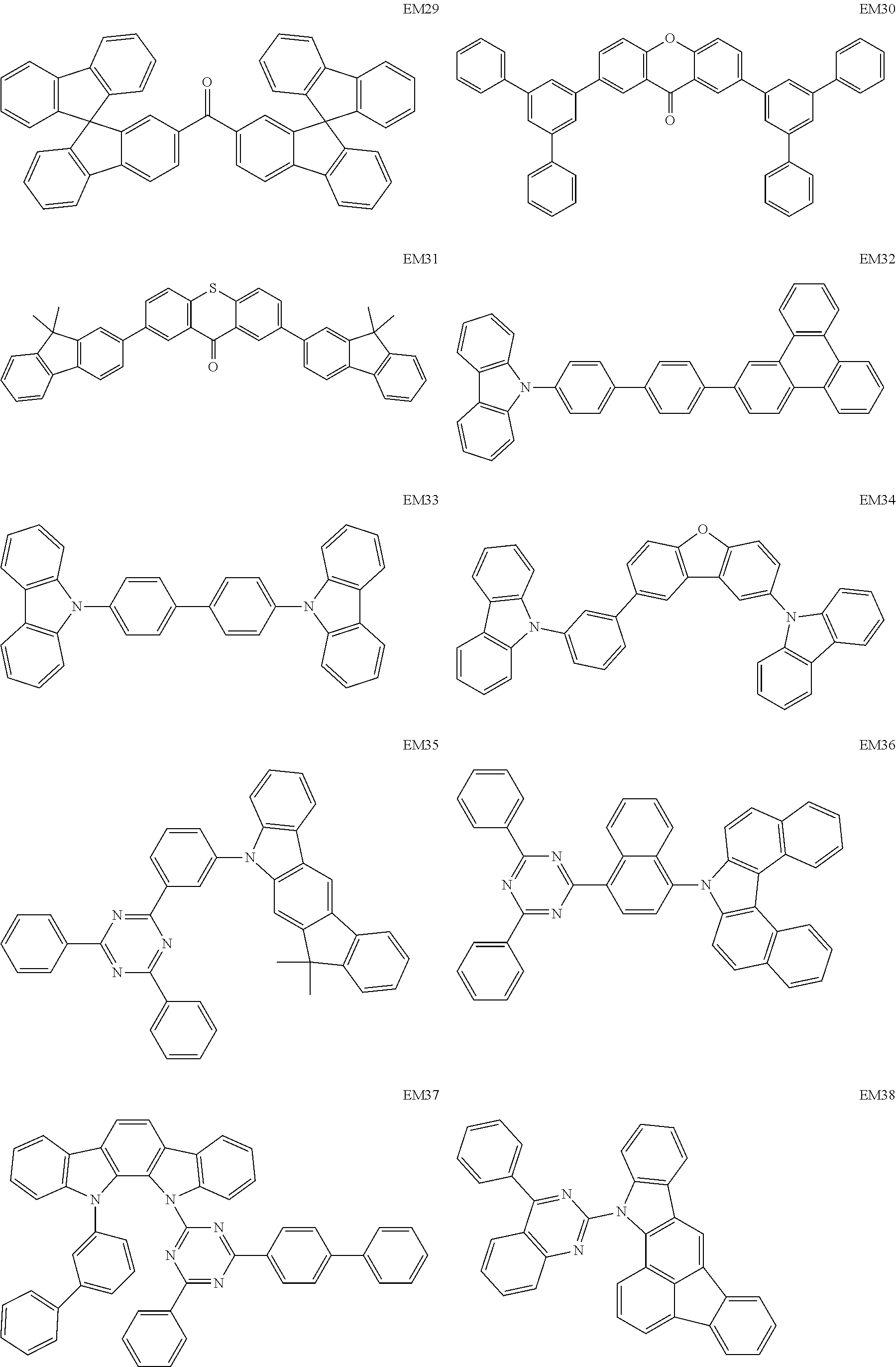

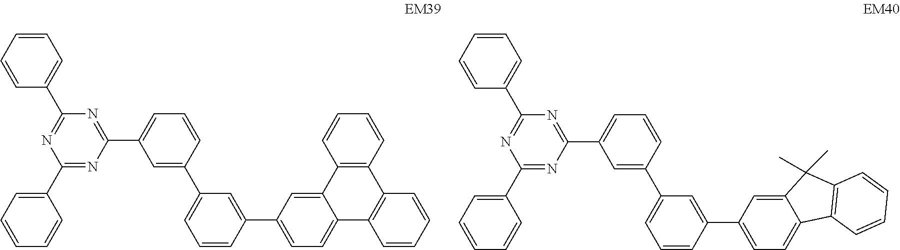

A compound containing a carbazole skeleton can be used as the assist material. Among the above specific examples, EM32 to EM38 can be used. A compound containing an azine ring can be used as the assist material. Among the above specific examples, EM35 to EM40 can be used. A compound containing a xanthone skeleton can be used as the assist material. Among the above specific examples, EM28 and EM30 can be used. A compound containing a thioxanthone skeleton can be used as the assist material. Among the above specific examples, EM31 can be used.

The electron transport material can be freely-selected from materials capable of transporting electrons injected from the cathode to the light-emitting layer and is selected in consideration of, for example, the balance with the hole mobility of the hole transport material. Examples of a material having the ability to transport electrons include oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused-ring compounds, such as fluorene derivatives, naphthalene derivatives, chrysene derivatives, and anthracene derivatives. The electron transport materials can be used for the hole-blocking layer.

Non-limiting specific examples of a compound used as the electron transport material will be described below.

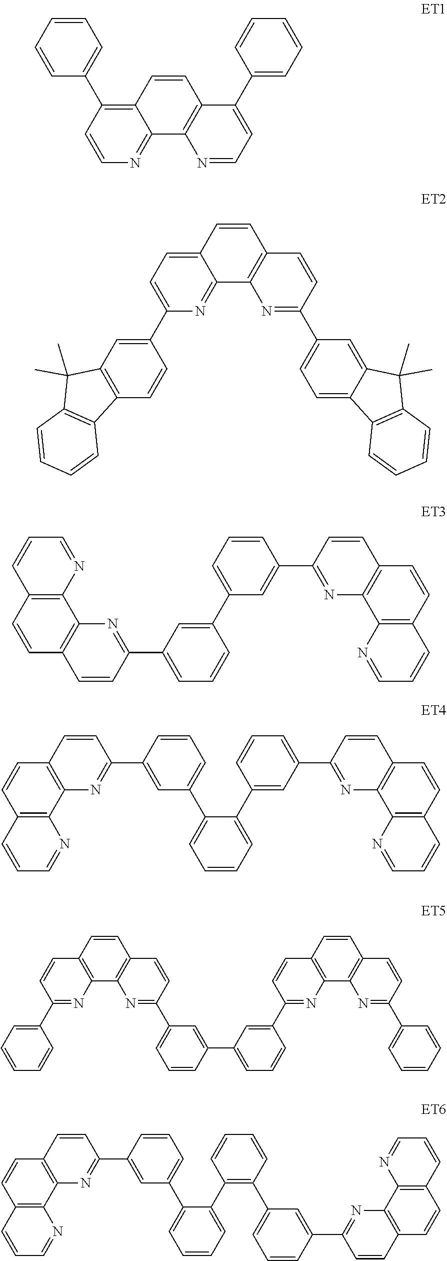

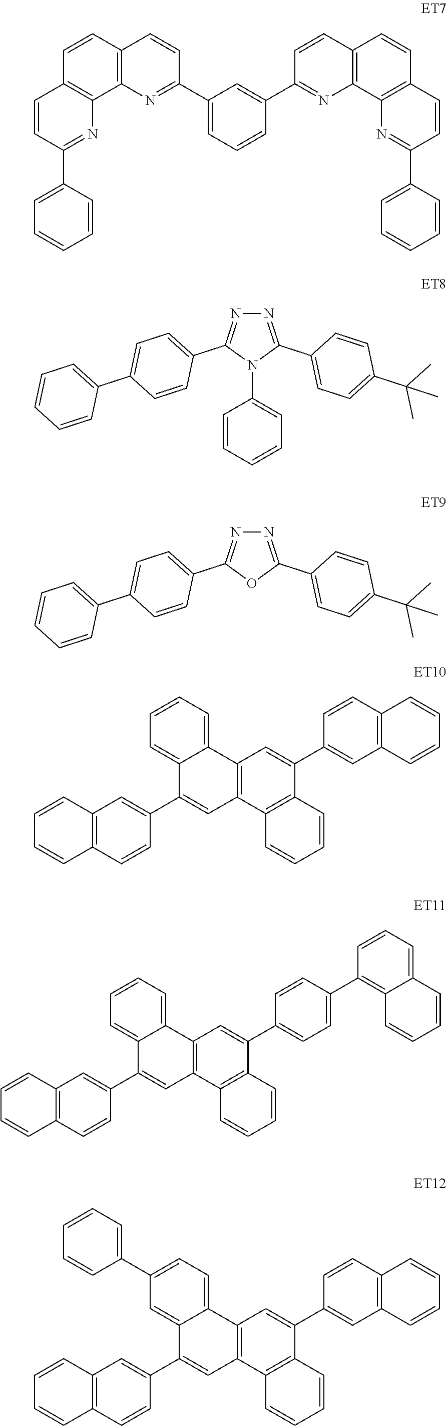

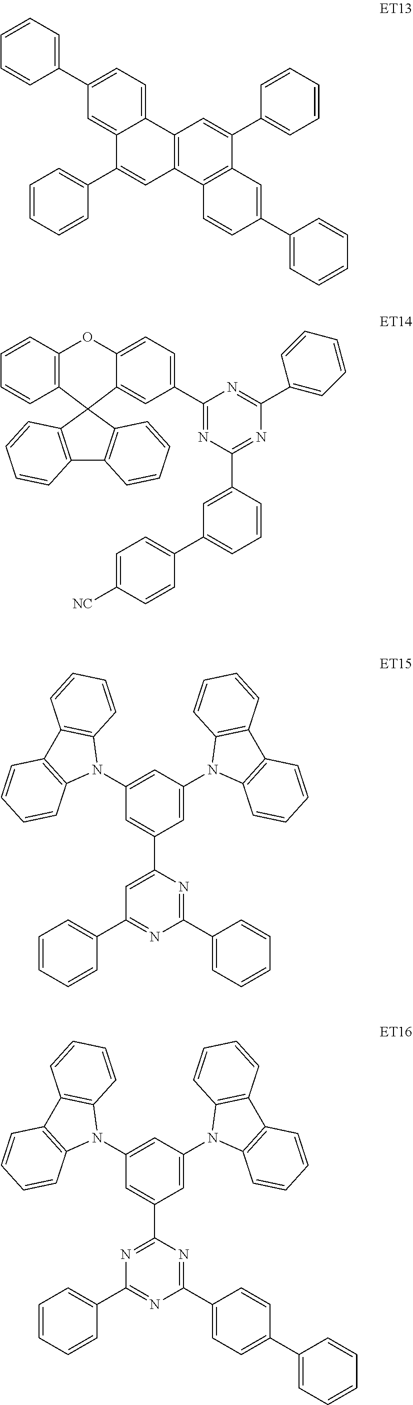

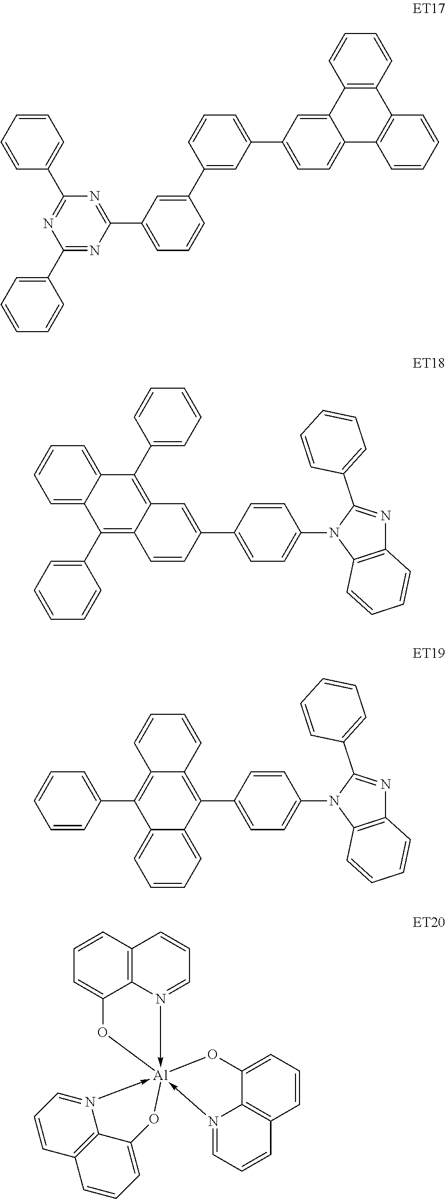

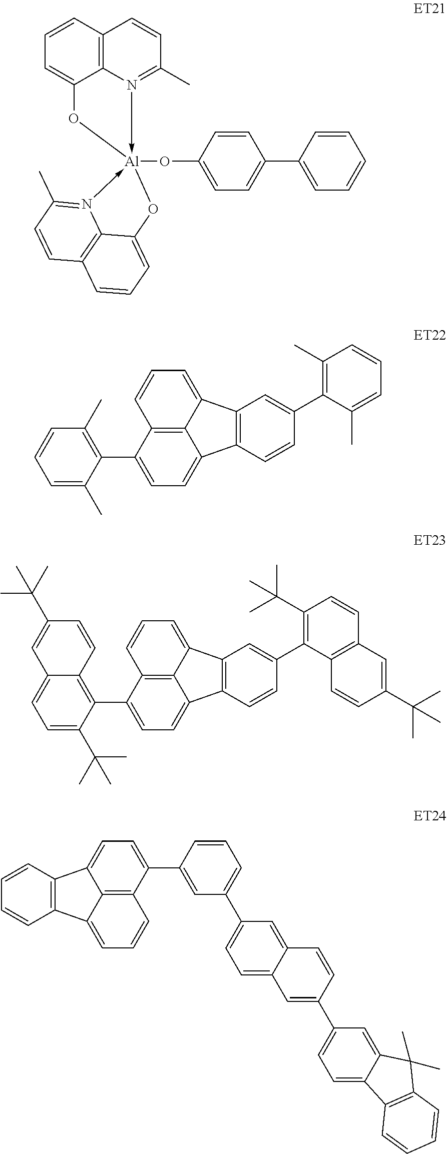

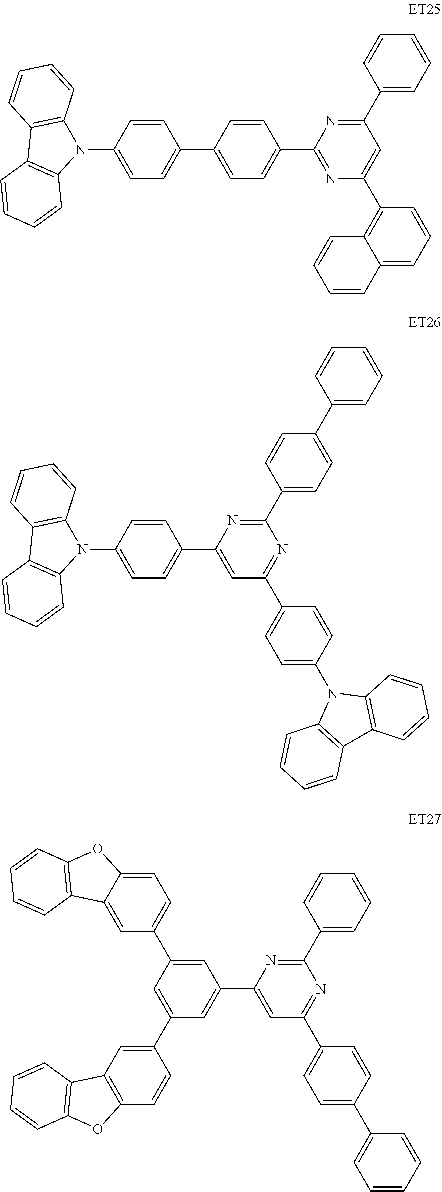 [SMILES: c1ccc(-c2cc(-c3cccc4ccccc34)nc(-c3ccc(-c4ccc(-n5c6ccccc6c6ccccc65)cc4)cc3)n2)cc1 ; c1ccc(-c2ccc(-c3nc(-c4ccc(-n5c6ccccc6c6ccccc65)cc4)cc(-c4ccc(-n5c6ccccc6c6ccccc65)cc4)n3)cc2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

An electron injection material can be freely-selected from materials capable of easily injecting electrons from the cathode and is selected in consideration of, for example, the balance with the hole injection properties. As the organic compound, n-type dopants and reducing dopants are also included. Examples thereof include alkali metal-containing compounds such as lithium fluoride, lithium complexes such as lithium quinolinolate, benzimidazolidene derivatives, imidazolidene derivatives, fulvalene derivatives, and acridine derivatives.

It can also be used in combination with the above-mentioned electron transport material.

### Configuration of Organic Light-Emitting Device

The organic light-emitting device includes a first electrode, an organic compound layer, and a second electrode over a substrate. An insulating layer may be provided over the substrate. A protective layer, a color filter, a microlens may be disposed over the second electrode. In the case of disposing the color filter, a planarization layer may be disposed between the protective layer and the color filter. The planarization layer can be composed of, for example, an acrylic resin. The same applies when a planarization layer is provided between the color filter and the microlens.

### Substrate

Examples of the substrate include silicon wafers, quartz substrates, glass substrates, resin substrates, and metal substrates. The substrate may include a switching element, such as a transistor, a line, and an insulating layer thereon. Any material can be used for the insulating layer as long as a contact hole can be formed in such a manner that a line can be coupled to the first electrode and as long as insulation with a non-connected line can be ensured. For example, a resin, such as polyimide, silicon oxide, or silicon nitride, can be used.

### Electrode

A pair of electrodes can be used. The pair of electrodes may be an anode and a cathode.

In the case where an electric field is applied in the direction in which the organic light-emitting device emits light, an electrode having a higher potential is the anode, and the other is the cathode. It can also be said that the electrode that supplies holes to the light-emitting layer is the anode and that the electrode that supplies electrons is the cathode.

As the component material of the anode, a material having a work function as high as possible can be used. Examples of the material that can be used include elemental metals, such as gold, platinum, silver, copper, nickel, palladium, cobalt, selenium, vanadium, and tungsten, mixtures thereof, alloys of combinations thereof, and metal oxides, such as tin oxide, zinc oxide, indium oxide, indium-tin oxide (ITO), and indium-zinc oxide. Additionally, conductive polymers, such as polyaniline, polypyrrole, and polythiophene, may be used.

These electrode materials may be used alone or in combination of two or more. The anode may be formed of a single layer or multiple layers.

When the anode is used as a reflective electrode, for example, chromium, aluminum, silver, titanium, tungsten, molybdenum, an alloy thereof, or a stack thereof may be used. These materials can also be used to act as a reflective film that does not have the role of an electrode. When the anode is used as a transparent electrode, a transparent conductive oxide layer composed of, for example, indium-tin oxide (ITO) or indium-zinc oxide may be used; however, the anode is not limited thereto.

The electrode can be formed by photolithography.

As the component material of the cathode, a material having a lower work function can be used. Examples thereof include elemental metals such as alkali metals, e.g., lithium, alkaline-earth metals, e.g., calcium, aluminum, titanium, manganese, silver, lead, and chromium, and mixtures thereof. Alloys of combinations of these elemental metals can also be used. For example, magnesium-silver, aluminum-lithium, aluminum-magnesium, silver-copper, and zinc-silver can be used. Metal oxides, such as indium-tin oxide (ITO), can also be used. These electrode materials may be used alone or in combination of two or more. The cathode may have a single-layer structure or a multilayer structure. In particular, silver can be used. To reduce the aggregation of silver, a silver alloy can be used. Any alloy ratio may be used as long as the aggregation of silver can be reduced. The ratio of silver to another metal may be, for example, 1:1 or 3:1.

A top emission device may be provided using the cathode formed of a conductive oxide layer composed of, for example, ITO. A bottom emission device may be provided using the cathode formed of a reflective electrode composed of, for example, aluminum (Al). Any type of cathode may be used. Any method for forming the cathode may be employed. For example, a direct-current or alternating-current sputtering technique can be employed because good film coverage is obtained and thus the resistance is easily reduced.

### Organic Compound Layer

The organic compound layer may be formed of a single layer or multiple layers. When multiple layers are present, they may be referred to as a hole injection layer, a hole transport layer, an electron-blocking layer, a light-emitting layer, a hole-blocking layer, an electron transport layer, or an electron injection layer in accordance with their functions. The organic compound layer is mainly composed of an organic compound, and may contain inorganic atoms and an inorganic compound. For example, the organic compound layer may contain, for example, copper, lithium, magnesium, aluminum, iridium, platinum, molybdenum, or zinc. The organic compound layer may be disposed between the first electrode and the second electrode, and may be disposed in contact with the first electrode and the second electrode.

The organic compound layer, such as the hole injection layer, the hole transport layer, the electron-blocking layer, the light-emitting layer, the hole-blocking layer, the electron transport layer, or the electron injection layer, included in the organic light-emitting device according to an embodiment of the present disclosure is formed by a method described below.

For the organic compound layer included in the organic light-emitting device according to an embodiment of the present disclosure, a dry process, such as a vacuum evaporation method, an ionized evaporation method, sputtering, or plasma, may be employed. Alternatively, instead of the dry process, it is also possible to employ a wet process in which a material is dissolved in an appropriate solvent and then a film is formed by a known coating method, such as spin coating, dipping, a casting method, a Langmuir-Blodgett (LB) technique, or an ink jet method.

When the layer is formed by, for example, the vacuum evaporation method or the solution coating method, crystallization and so forth are less likely to occur, and good stability with time is obtained. In the case of forming a film by the coating method, the film may be formed in combination with an appropriate binder resin.

Non-limiting examples of the binder resin include poly(vinyl carbazole) resins, polycarbonate resins, polyester resins, acrylonitrile butadiene styrene (ABS) resins, acrylic resins, polyimide resins, phenolic resins, epoxy resins, silicone resins, and urea resins.

These binder resins may be used alone as a homopolymer or copolymer or in combination as a mixture of two or more. Furthermore, additives, such as a known plasticizer, antioxidant, and ultraviolet absorber, may be used, as needed.

### Protective Layer

A protective layer may be disposed on the cathode. For example, a glass member provided with a moisture absorbent can be bonded to the cathode to reduce the entry of, for example, water into the organic compound layer, thereby reducing the occurrence of display defects. In another embodiment, a passivation film composed of, for example, silicon nitride may be disposed on the cathode to reduce the entry of, for example, water into the organic compound layer. For example, after the formation of the cathode, the substrate may be transported to another chamber without breaking the vacuum, and a silicon nitride film having a thickness of 2 μm may be formed by a chemical vapor deposition (CVD) method to provide a protective layer. After the film deposition by the CVD method, a protective layer may be formed by an atomic layer deposition (ALD) method. Non-limiting examples of the material of the layer formed by the ALD method may include silicon nitride, silicon oxide, and aluminum oxide. Silicon nitride may be deposited by the CVD method on the layer formed by the ALD method. The film formed by the ALD method may have a smaller thickness than the film formed by the CVD method. Specifically, the film thickness may be 50% or less, even 10% or less.

### Color Filter

A color filter may be disposed on the protective layer. For example, a color filter may be disposed on another substrate in consideration of the size of the organic light-emitting device and bonded to the substrate provided with the organic light-emitting device. A color filter may be formed by patterning on the protective layer using photolithography. The color filter may be composed of a polymer.

### Planarization Layer

A planarization layer may be disposed between the color filter and the protective layer. The planarization layer is provided for the purpose of reducing the unevenness of the layer underneath. The planarization layer may be referred to as a “material resin layer” without limiting its purpose. The planarization layer may be composed of an organic compound. A low- or high-molecular-weight organic compound may be used. A high-molecular-weight organic compound can be used.

The planarization layers may be disposed above and below (or on) the color filter and may be composed of the same or different component materials. Specific examples thereof include poly(vinyl carbazole) resins, polycarbonate resins, polyester resins, acrylonitrile butadiene styrene (ABS) resins, acrylic resins, polyimide resins, phenolic resins, epoxy resins, silicone resins, and urea resins.

### Microlens

An organic light-emitting apparatus may include an optical member, such as a microlens, on the outgoing light side. The microlens can be composed of, for example, an acrylic resin or an epoxy resin. The microlens may be used to increase the amount of light emitted from the organic light-emitting apparatus and to control the direction of the light emitted. The microlens may have a hemispherical shape. In the case of a hemispherical shape, among tangents to the hemisphere, there is a tangent parallel to the insulating layer. The point of contact of the tangent with the hemisphere is the vertex of the microlens. The vertex of the microlens can be determined in the same way for any cross-sectional view. That is, among the tangents to the semicircle of the microlens in the cross-sectional view, there is a tangent parallel to the insulating layer, and the point of contact of the tangent with the semicircle is the vertex of the microlens.

The midpoint of the microlens can be defined. In the cross section of the microlens, when a segment is hypothetically drawn from the point where an arc shape ends to the point where another arc shape ends, the midpoint of the segment can be referred to as the midpoint of the microlens. The cross section to determine the vertex and midpoint may be a cross section perpendicular to the insulating layer.

### Opposite Substrate

An opposite substrate may be disposed on the planarization layer. The opposite substrate is disposed at a position corresponding to the substrate described above and thus is called an opposite substrate. The opposite substrate may be composed of the same material as the substrate described above. When the above-described substrate is referred to as a first substrate, the opposite substrate may be referred to as a second substrate.

### Pixel Circuit

An light-emitting apparatus including organic light-emitting devices may include pixel circuits coupled to the organic light-emitting devices. Each of the pixel circuits may be of an active matrix type, which independently controls the emission of first and second light-emitting devices. The active matrix type circuit may be voltage programming or current programming. A driving circuit includes the pixel circuit for each pixel. The pixel circuit may include a light-emitting device, a transistor to control the luminance of the light-emitting device, a transistor to control the timing of the light emission, a capacitor to retain the gate voltage of the transistor to control the luminance, and a transistor to connect to GND without using the light-emitting device.

The light-emitting apparatus includes a display area and a peripheral area disposed around the display area. The display area includes a pixel circuit, and the peripheral area includes a display control circuit. The mobility of a transistor contained in the pixel circuit may be lower than the mobility of a transistor contained in the display control circuit.

The gradient of the current-voltage characteristics of the transistor contained in the pixel circuit may be smaller than the gradient of the current-voltage characteristic of the transistor contained in the display control circuit. The gradient of the current-voltage characteristics can be measured by what is called Vg-Ig characteristics.

The transistor contained in the pixel circuit is a transistor coupled to a light-emitting device, such as a first light-emitting device.

Pixel

The organic light-emitting apparatus includes multiple pixels. Each pixel includes subpixels configured to emit colors different from each other. The subpixels may have respective red, green, and blue (RGB) emission colors.

Light emerges from a region of the pixel, also called a pixel aperture. This region is the same as a first region.

The pixel aperture may be 15 μm or less, and may be 5 μm or more. More specifically, the pixel aperture may be, for example, 11 μm, 9.5 μm, 7.4 μm, or 6.4 μm.

The distance between subpixels may be 10 μm. Specifically, the distance may be 8 μm, 7.4 μm, or 6.4 μm.

The pixels may be arranged in a known pattern in plan view. For example, a stripe pattern, a delta pattern, a Pen Tile matrix pattern, or the Bayer pattern may be used. The shape of each subpixel in plan view may be any known shape. Examples of the shape of the subpixel include quadrilaterals, such as rectangles and rhombi, and hexagons. Of course, if the shape is close to a rectangle, rather than an exact shape, it is included in the rectangle. The shape of the subpixel and the pixel arrangement can be used in combination.

### Application of Organic Light-Emitting Device According to an Embodiment of the Present Disclosure

The organic light-emitting device according to an embodiment of the present disclosure can be used as a component member of a display apparatus or lighting apparatus. Other applications include exposure light sources for electrophotographic image-forming apparatuses, backlights for liquid crystal display apparatuses, and light-emitting apparatuses including white-light sources and color filters.

The display apparatus may be an image information-processing unit having an image input unit that receives image information from an area or linear CCD sensor, a memory card, or any other source, an information-processing unit that processes the input information, and a display unit that displays the input image. The display apparatus includes multiple pixels, and at least one of the multiple pixels may include the organic light-emitting device according to the present embodiment and a transistor coupled to the organic light-emitting device. In this case, the substrate may be a semiconductor substrate composed of, for example silicon, and the transistor may be a MOSFET formed on or at the substrate.

The display unit of an image pickup apparatus or an inkjet printer may have a touch panel function. The driving mode of the touch panel function may be, but is not particularly limited to, an infrared mode, an electrostatic capacitance mode, a resistive film mode, or an electromagnetic inductive mode. The display apparatus may also be used for a display unit of a multifunction printer.

The following describes a display apparatus according to the present embodiment with reference to the attached drawings.

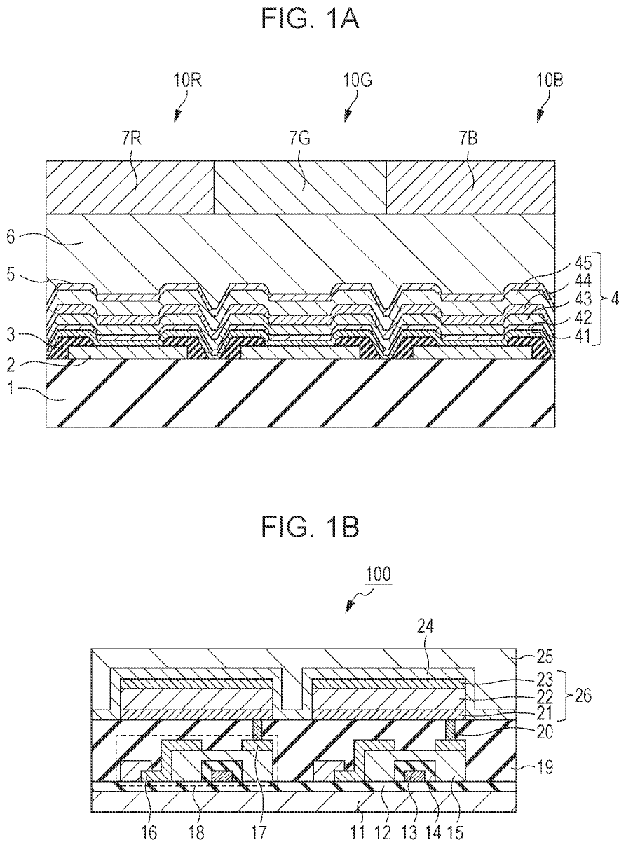

FIGS. 1A and 1B are each a schematic cross-sectional view of an example of a display apparatus including organic light-emitting devices and transistors coupled to the respective organic light-emitting devices. Each of the transistors is an example of an active element. The transistors may be thin-film transistors (TFTs).

FIG. 1A is an example of pixels that are components of the display apparatus according to the present embodiment. Each of the pixels includes subpixels 10. The subpixels are divided into 10R, 10G, and 10B according to their light emission. The emission colors may be distinguished by the wavelength of light emitted from the light-emitting layer. Light emitted from the subpixels may be selectively transmitted or color-converted with, for example, a color filter. Each subpixels includes a reflective electrode 2 serving as a first electrode, an insulating layer 3 covering the edge of the reflective electrode 2, an organic compound layer 4 covering the first electrode and the insulating layer, a transparent electrode 5, a protective layer 6, and a color filter 7, over an interlayer insulating layer 1.

The transistors and capacitive elements may be disposed under or in the interlayer insulating layer 1.

Each transistor may be electrically coupled to a corresponding one of the first electrodes through a contact hole (not illustrated).

The insulating layer 3 is also called a bank or pixel separation film. The insulating layer covers the edge of each first electrode and surrounds the first electrode. Portions that are not covered with the insulating layer are in contact with the organic compound layer 4 and serve as light-emitting regions.

The organic compound layer 4 includes a hole injection layer 41, a hole transport layer 42, a first light-emitting layer 43, a second light-emitting layer 44, and an electron transport layer 45.

The second electrode 5 may be a transparent electrode, a reflective electrode, or a semi-transparent electrode.

The protective layer 6 reduces the penetration of moisture into the organic compound layer. Although the protective layer is illustrated as a single layer, the protective layer may include multiple layers, and each layer may be an inorganic compound layer or an organic compound layer.

The color filter 7 is separated into 7R, 7G, and 7B according to its color. The color filter may be disposed on a planarizing film (not illustrated). A resin protective layer (not illustrated) may be disposed on the color filter. The color filter may be disposed on the protective layer 6. Alternatively, the color filter may be disposed on an opposite substrate, such as a glass substrate, and then bonded.

A display apparatus 100 illustrated in FIG. 1B includes organic light-emitting devices 26 and TFTs 18 as an example of transistors. A substrate 11 composed of a material, such as glass or silicon, is provided, and an insulating layer 12 is disposed thereon. Active elements, such as the TFTs 18, are disposed on the insulating layer 12. The gate electrode 13, the gate insulating film 14, and the semiconductor layer 15 of each of the active elements are disposed thereon. Each TFT 18 further includes a semiconductor layer 15, a drain electrode 16, and a source electrode 17. The TFTs 18 are overlaid with an insulating film 19. Anode 21 included in the organic light-emitting devices 26 is coupled to the source electrodes 17 through contact holes 20 provided in the insulating film.

The mode of electrical connection between the electrodes (anode 21 and cathode 23) included in each organic light-emitting device 26 and the electrodes (source electrode 17 and drain electrode 16) included in a corresponding one of the TFTs is not limited to the mode illustrated in FIG. 1B. That is, it is sufficient that any one of the anode 21 and the cathode 23 is electrically coupled to any one of the source electrode 17 and the drain electrode 16 of the TFT 18.

In the display apparatus 100 illustrated in FIG. 1B, although each organic compound layer 22 is illustrated as a single layer, the organic compound layer 22 may include multiple layers. To reduce the deterioration of the organic light-emitting devices, a first protective layer 24 and a second protective layer 25 are disposed on the cathodes 23.

In the display apparatus 100 illustrated in FIG. 1B, although the transistors are used as switching elements, other switching elements, such as MIM elements, may be used instead.

The transistors used in the display apparatus 100 illustrated in FIG. 1B are not limited to transistors using a single-crystal silicon wafer, but may also be thin-film transistors including active layers on the insulating surface of a substrate. Examples of the material of the active layers include single-crystal silicon, non-single-crystal silicon, such as amorphous silicon and microcrystalline silicon; and non-single-crystal oxide semiconductors, such as indium zinc oxide and indium gallium zinc oxide. Thin-film transistors are also called TFT elements.

The transistors in the display apparatus 100 illustrated in FIG. 1B may be formed in the substrate, such as a S1 substrate. The expression “formed in the substrate” indicates that the transistors are produced by processing the substrate, such as a S1 substrate. In the case where the transistors are formed in the substrate, the substrate and the transistors can be deemed to be integrally formed.

In the organic light-emitting device according to the present embodiment, the luminance is controlled by the TFT devices, which are an example of switching elements; thus, an image can be displayed at respective luminance levels by arranging multiple organic light-emitting devices in the plane. The switching devices according to the present embodiment are not limited to the TFT devices and may be low-temperature polysilicon transistors or active-matrix drivers formed on a substrate such as a S1 substrate. The expression “on a substrate” can also be said to be “in the substrate”. Whether transistors are formed in the substrate or TFT devices are used is selected in accordance with the size of a display unit. For example, in the case where the display unit has a size of about 0.5 inches, organic light-emitting devices can be disposed on a S1 substrate.

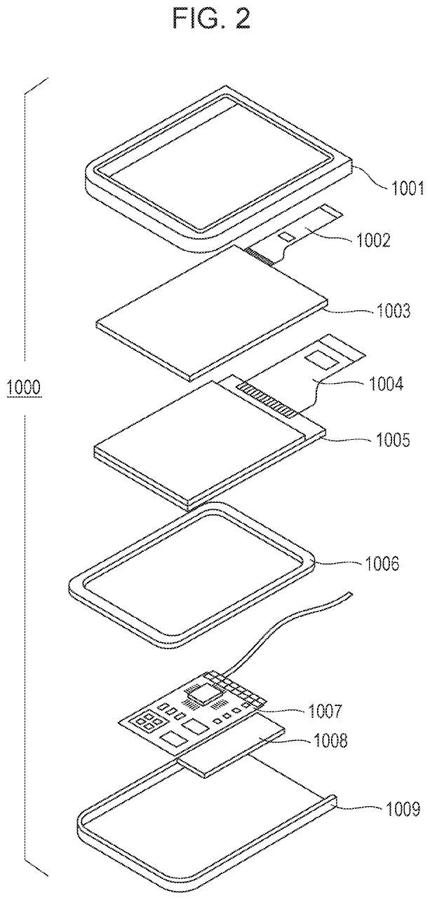

FIG. 2 is a schematic view illustrating an example of a display apparatus according to the present embodiment. A display apparatus 1000 may include a touch panel 1003, a display panel 1005, a frame 1006, a circuit substrate 1007, and a battery 1008 disposed between an upper cover 1001 and a lower cover 1009. The touch panel 1003 and the display panel 1005 are coupled to flexible printed circuits FPCs 1002 and 1004, respectively. The circuit substrate 1007 includes printed transistors. The battery 1008 need not be provided unless the display apparatus is a portable apparatus. The battery 1008 may be disposed at a different position even if the display apparatus is a portable apparatus.

The display apparatus according to the present embodiment may include a color filter having red, green, and blue portions. In the color filter, the red, green, and blue portions may be arranged in a delta arrangement.

The display apparatus according to the present embodiment may be used for the display unit of a portable terminal. In that case, the display apparatus may have both a display function and an operation function. Examples of the portable terminal include mobile phones such as smartphones, tablets, and head-mounted displays.

The display apparatus according to the present embodiment may be used for a display unit of an image pickup apparatus including an optical unit including multiple lenses and an image pickup device that receives light passing through the optical unit. The image pickup apparatus may include a display unit that displays information acquired by the image pickup device. The display unit may be a display unit exposed to the outside of the image pickup apparatus or a display unit disposed in a finder. The image pickup apparatus may be a digital camera or a digital camcorder.

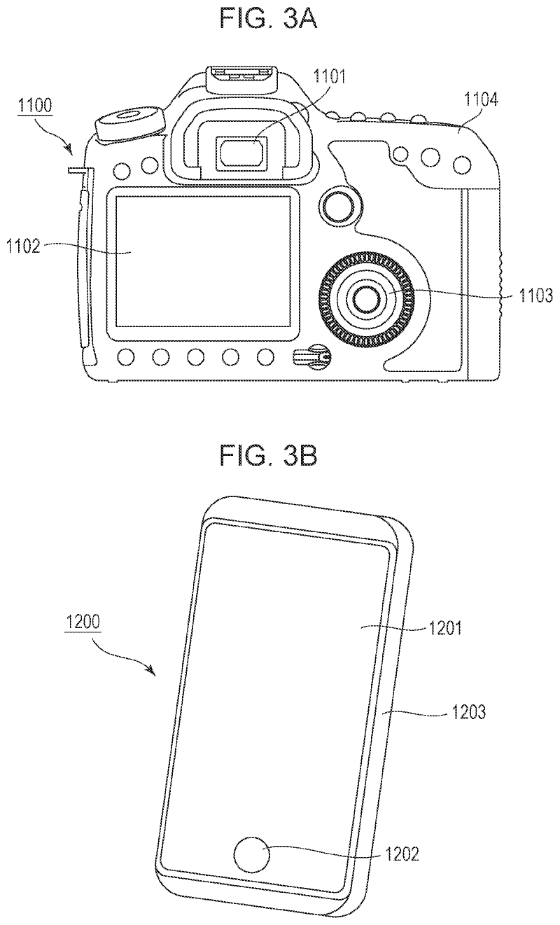

FIG. 3A is a schematic view illustrating an example of an image pickup apparatus according to the present embodiment. An image pickup apparatus 1100 may include a viewfinder 1101, a rear display 1102, an operation unit 1103, and a housing 1104. The viewfinder 1101 may include the display apparatus according to the present embodiment. In this case, the display apparatus may display environmental information, imaging instructions, and so forth in addition to an image to be captured. The environmental information may include, for example, the intensity of external light, the direction of external light, the moving speed of a subject, and the possibility that a subject is shielded by a shielding material.

The timing suitable for imaging is only for a short time. It is thus better to display the information as soon as possible. Accordingly, the display apparatus including the organic light-emitting device according to the present embodiment can be used.

This is because the organic light-emitting device has a high response speed. Display apparatuses including organic light-emitting devices are required to have a high display speed, and these apparatuses can be used more suitably than liquid crystal display apparatuses.

The image pickup apparatus 1100 includes an optical unit (not illustrated). The optical unit includes multiple lenses and is configured to form an image on an image pickup device in the housing 1104. The relative positions of the multiple lenses can be adjusted to adjust the focal point. This operation can also be performed automatically. The image pickup apparatus may translate to a photoelectric conversion apparatus. Examples of an image capturing method employed in the photoelectric conversion apparatus may include a method for detecting a difference from the previous image and a method of cutting out an image from images always recorded, instead of sequentially capturing images.

FIG. 3B is a schematic view illustrating an example of an electronic apparatus according to the present embodiment. An electronic apparatus 1200 includes a display unit 1201, an operation unit 1202, and a housing 1203. The housing 1203 may accommodate a circuit, a printed circuit board including the circuit, a battery, and a communication unit. The operation unit 1202 may be a button or a touch-screen-type reactive unit. The operation unit may be a biometric recognition unit that recognizes a fingerprint to release the lock or the like. An electronic apparatus having a communication unit can also be referred to as a communication apparatus. The electronic apparatus may further have a camera function by being equipped with a lens and an image pickup device. An image captured by the camera function is displayed on the display unit. Examples of the electronic apparatus include smartphones and notebook computers.

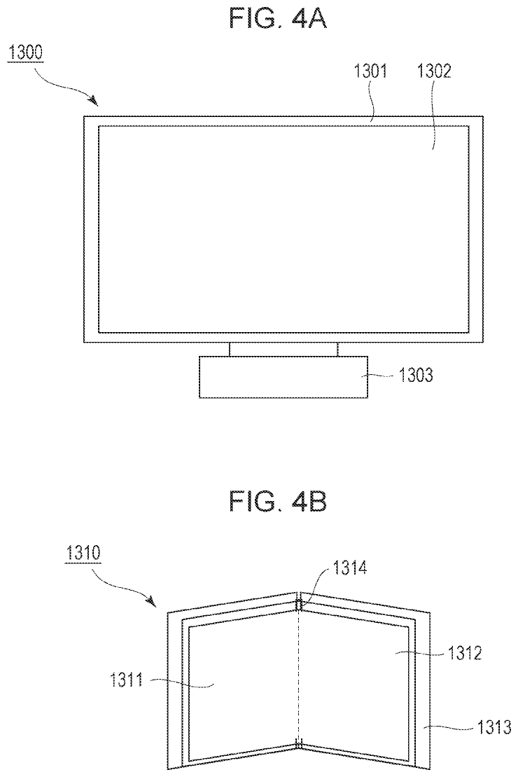

FIG. 4A is a schematic view illustrating an example of the display apparatus according to the present embodiment. FIG. 4A illustrates a display apparatus, such as a television monitor or a PC monitor. A display apparatus 1300 includes a frame 1301 and a display unit 1302. The light-emitting apparatus according to the present embodiment may be used for the display unit 1302.

The display apparatus 1300 includes a base 1303 that supports the frame 1301 and the display unit 1302. The base 1303 is not limited to the structure illustrated in FIG. 4A. The lower side of the frame 1301 may also serve as a base.

The frame 1301 and the display unit 1302 may be curved. These may have a radius of curvature of 5,000 mm or more and 6,000 mm or less.

FIG. 4B is a schematic view illustrating another example of a display apparatus according to the present embodiment. A display apparatus 1310 illustrated in FIG. 4B can be folded and is what is called a foldable display apparatus. The display apparatus 1310 includes a first display portion 1311, a second display portion 1312, a housing 1313, and an inflection point 1314. The first display portion 1311 and the second display portion 1312 may include the light-emitting apparatus according to the present embodiment. The first display portion 1311 and the second display portion 1312 may be a single, seamless display apparatus. The first display portion 1311 and the second display portion 1312 can be divided from each other at the inflection point. The first display portion 1311 and the second display portion 1312 may display different images. Alternatively, a single image may be displayed in the first and second display portions.

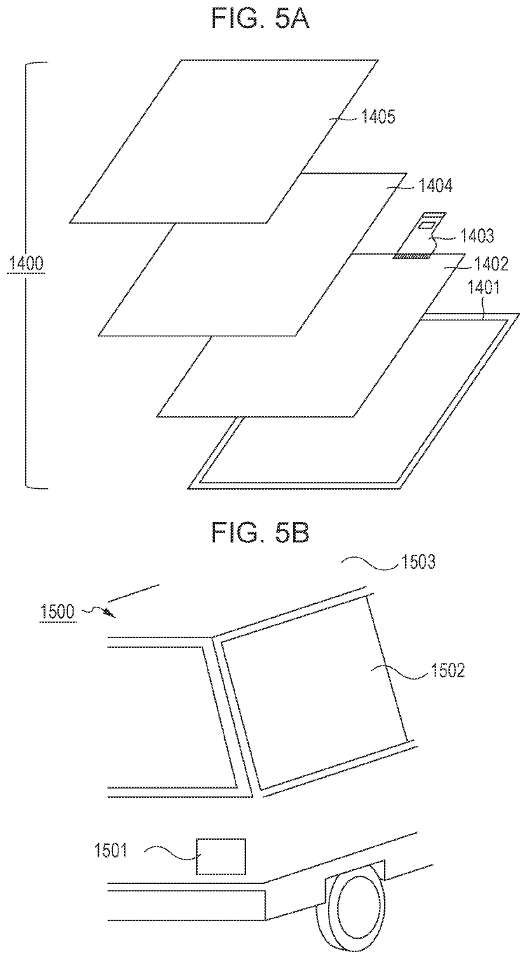

FIG. 5A is a schematic view illustrating an example of a lighting apparatus according to the present embodiment. A lighting apparatus 1400 may include a housing 1401, a light source 1402, a circuit board 1403, an optical film 1404, and a light diffusion unit 1405. The light source 1402 may include an organic light-emitting device according to the present embodiment. The optical film may be a film that improves the color rendering properties of the light source. The light diffusion unit can effectively diffuse light from the light source to deliver the light to a wide range when used for illumination and so forth. The optical film and the light diffusion unit may be disposed at the light emission side of the lighting apparatus. A cover may be disposed at the outermost portion, as needed.

The lighting apparatus is, for example, an apparatus that lights a room. The lighting apparatus may emit light of white, neutral white, or any color from blue to red. A light control circuit configured to control the light and a color control circuit configured to control the emission color may be provided. The lighting apparatus may include the organic light-emitting device according to an embodiment of the present disclosure and a power supply circuit coupled thereto. The power supply circuit is a circuit that converts an AC voltage into a DC voltage. The color temperature of white is 4,200 K, and the color temperature of neutral white is 5,000 K. The lighting apparatus may include a color filter.

The lighting apparatus according to the present embodiment may include a heat dissipation unit. The heat dissipation unit is configured to release heat in the apparatus to the outside of the apparatus and is composed of, for example, a metal having a high specific heat and liquid silicone.

FIG. 5B is a schematic view illustrating an automobile as an example of a moving object according to the present embodiment. The automobile includes a tail lamp, which is an example of lighting units. An automobile 1500 includes a tail lamp 1501 and may be configured to light the tail lamp when a brake operation or the like is performed.

The tail lamp 1501 may include an organic light-emitting device according to the present embodiment. The tail lamp 1501 may include a protective member that protects the organic light-emitting device. The protective member may be composed of any transparent material having high strength to some extent and can be composed of, for example, polycarbonate. The polycarbonate may be mixed with, for example, a furandicarboxylic acid derivative or an acrylonitrile derivative.

The automobile 1500 may include an automobile body 1503 and windows 1502 attached thereto. The windows 1502 may be transparent displays if the windows are not used to check the front and back of the automobile. The transparent displays may include an organic light-emitting device according to the present embodiment.

In this case, the components, such as the electrodes, of the organic light-emitting device are formed of transparent members.

The moving object according to the present embodiment may be, for example, a ship, an aircraft, or a drone. The moving object may include a body and a lighting unit attached to the body. The lighting unit may emit light to indicate the position of the body. The lighting unit includes the organic light-emitting device according to the embodiment.

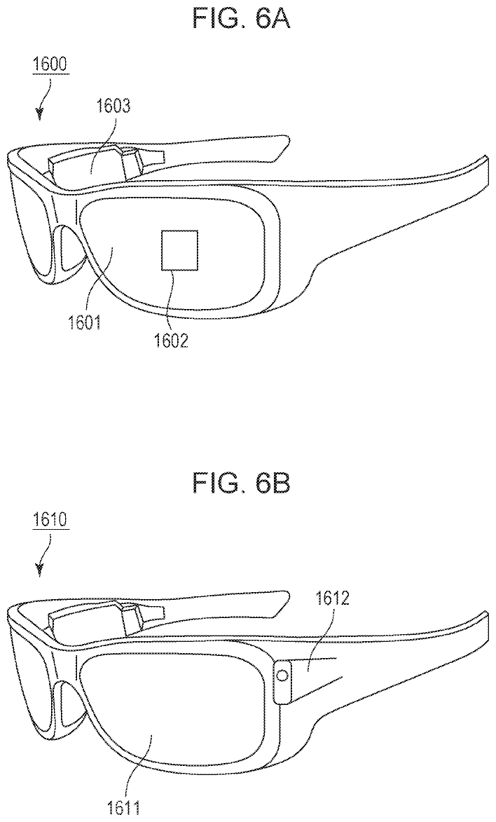

Examples of applications of the display apparatuses of the above embodiments will be described with reference to FIGS. 6A and 6B. The display apparatuses can be used for systems that can be worn as wearable devices, such as smart glasses, head-mounted displays (HMDs), and smart contacts. An image pickup and display apparatus used in such an example of the applications has an image pickup apparatus that can photoelectrically convert visible light and a display apparatus that can emit visible light.

FIG. 6A illustrates glasses 1600 (smart glasses) according to an example of applications. An image pickup apparatus 1602, such as a complementary metal-oxide semiconductor (CMOS) sensor or a single-photon avalanche diode (SPAD), is provided on a front side of a lens 1601 of the glasses 1600. The display apparatus according to any of the above-mentioned embodiments is provided on the back side of the lens 1601.

The glasses 1600 further include a control unit 1603. The control unit 1603 functions as a power source that supplies electric power to the image pickup apparatus 1602 and the display apparatus according to any of the embodiments. The control unit 1603 controls the operation of the image pickup apparatus 1602 and the display apparatus. The lens 1601 has an optical system for focusing light on the image pickup apparatus 1602.

FIG. 6B illustrates glasses 1610 (smart glasses) according to an example of applications. The glasses 1610 include a control unit 1612. The control unit 1612 includes an image pickup apparatus corresponding to the image pickup apparatus 1602 and a display apparatus. A lens 1611 is provided with the image pickup apparatus in the control unit 1612 and an optical system that projects light emitted from the display apparatus. An image is projected onto the lens 1611. The control unit 1612 functions as a power source that supplies electric power to the image pickup apparatus and the display apparatus and controls the operation of the image pickup apparatus and the display apparatus. The control unit may include a gaze detection unit that detects the gaze of a wearer. Infrared light may be used for gaze detection. An infrared light-emitting unit emits infrared light to an eyeball of a user who is gazing at a displayed image. An image of the eyeball is captured by detecting the reflected infrared light from the eyeball with an image pickup unit having light-receiving elements. The deterioration of image quality is reduced by providing a reduction unit configured to reduce light from the infrared light-emitting unit to the display unit when viewed in plan.

The user's gaze at the displayed image is detected from the image of the eyeball captured with the infrared light. Any known method can be used to the gaze detection using the captured image of the eyeball. As an example, a gaze detection method based on a Purkinje image of the reflection of irradiation light on a cornea can be used.

More specifically, the gaze detection process is based on a pupil-corneal reflection method. Using the pupil-corneal reflection method, the user's gaze is detected by calculating a gaze vector representing the direction (rotation angle) of the eyeball based on the image of the pupil and the Purkinje image contained in the captured image of the eyeball.

A display apparatus according to an embodiment of the present disclosure may include an image pickup apparatus including light-receiving elements, and may control an image displayed on the display apparatus based on the gaze information of the user from the image pickup apparatus.

Specifically, in the display apparatus, a first field-of-view area at which the user gazes and a second field-of-view area other than the first field-of-view area are determined on the basis of the gaze information. The first field-of-view area and the second field-of-view area may be determined by the control unit of the display apparatus or may be determined by receiving those determined by an external control unit. In the display area of the display apparatus, the display resolution of the first field-of-view area may be controlled to be higher than the display resolution of the second field-of-view area. That is, the resolution of the second field-of-view area may be lower than that of the first field-of-view area.

The display area includes a first display area and a second display area different from the first display area. Based on the gaze information, an area of higher priority is determined from the first display area and the second display area. The first display area and the second display area may be determined by the control unit of the display apparatus or may be determined by receiving those determined by an external control unit. The resolution of an area of higher priority may be controlled to be higher than the resolution of an area other than the area of higher priority. In other words, the resolution of an area of a relatively low priority may be low.

Artificial intelligence (AI) may be used to determine the first field-of-view area or the high-priority area. The AI may be a model configured to estimate the angle of gaze from the image of the eyeball and the distance to a target object located in the gaze direction, using the image of the eyeball and the actual direction of gaze of the eyeball in the image as teaching data. The AI program may be stored in the display apparatus, the image pickup apparatus, or an external apparatus. When the AI program is stored in the external apparatus, the AI program is transmitted to the display apparatus via communications.

In the case of controlling the display based on visual detection, it can be applied to smart glasses that further include an image pickup apparatus configured to capture an image of the outside. The smart glasses can display captured external information in real time.

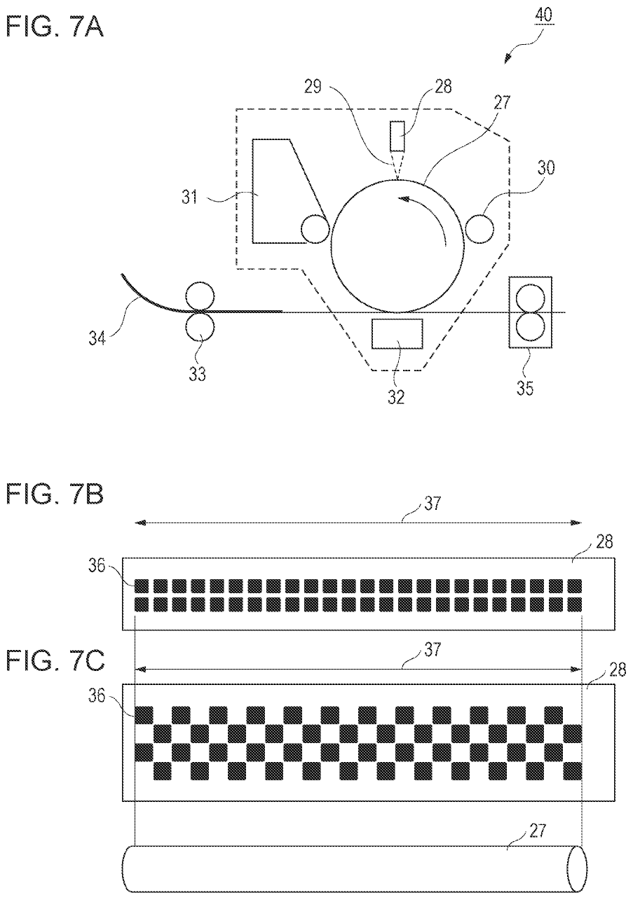

FIG. 7A is a schematic view of an example of an image-forming apparatus according to an embodiment of the present disclosure. An image-forming apparatus 40 is an electrophotographic image-forming apparatus and includes a photoconductor 27, an exposure light source 28, a charging unit 30, a developing unit 31, a transfer unit 32, a transport roller 33, and a fusing unit 35. The irradiation of light 29 is performed from the exposure light source 28 to form an electrostatic latent image on the surface of the photoconductor 27. The exposure light source 28 includes the organic light-emitting device according to the present embodiment. The developing unit 31 contains, for example, a toner. The charging unit 30 charges the photoconductor 27. The transfer unit 32 transfers the developed image to a recording medium 34. The transport roller 33 transports the recording medium 34. The recording medium 34 is paper, for example. The fusing unit 35 fixes the image formed on the recording medium 34.

FIGS. 7B and 7C each illustrate the exposure light source 28 and are each a schematic view illustrating multiple light-emitting portions 36 arranged on a long substrate. Arrows 37 each represent the row direction in which the organic light-emitting devices are arranged. The row direction is the same as the direction of the axis on which the photoconductor 27 rotates. This direction can also be referred to as the long-axis direction of the photoconductor 27. FIG. 7B illustrates a configuration in which the light-emitting portions 36 are arranged in the long-axis direction of the photoconductor 27. FIG. 7C is different from FIG. 7B in that the light-emitting portions 36 are arranged alternately in the row direction in a first row and a second row. The first row and the second row are located at different positions in the column direction. In the first row, the multiple light-emitting portions 36 are spaced apart. The second row has the light-emitting portions 36 at positions corresponding to the positions between the light-emitting portions 36 in the first row. In other words, the multiple light-emitting portions 36 are also spaced apart in the column direction. The arrangement in FIG. 7C can be rephrased as, for example, a lattice arrangement, a staggered arrangement, or a checkered pattern.

As described above, the use of an apparatus including the organic light-emitting device according to the present embodiment enables a stable display with good image quality even for a long time.

### EXAMPLES

The present disclosure will be described below by examples. However, the invention is not limited thereto.

### Example 1: Synthesis of Exemplified Compound A1

Exemplified compound A1 was synthesized according to the following scheme.

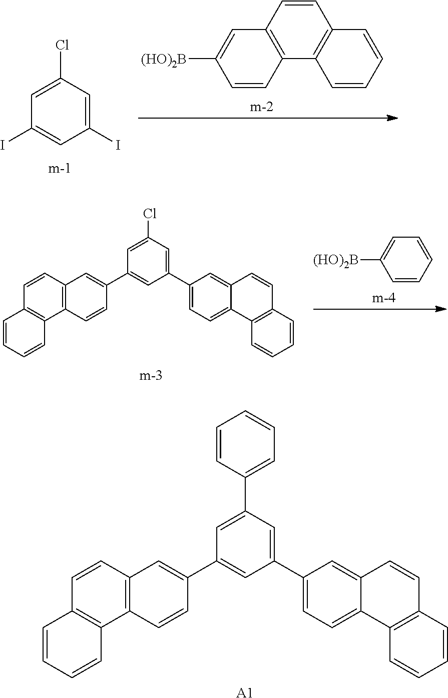

### (1) Synthesis of Compound m-3

The following reagents and solvents were placed in a 200-mL recovery flask.

Compound m-1: 2.00 g (5.49 mmol)
Compound m-2: 2.68 g (12.1 mmol)
Pd(PPh₃)₄: 0.32 g

### Toluene: 100 ml

### Ethanol: 20 ml

2 M aqueous sodium carbonate solution: 20 mL

The reaction solution was heated to reflux with stirring for 6 hours under a stream of nitrogen. After completion of the reaction, water was added thereto, and then liquid-liquid extraction was performed. Dissolution was performed with chloroform. Purification was performed by column chromatography (chloroform/heptane), and then recrystallization was performed in toluene/heptane to give 1.91 g (yield: 75%) of compound m-3 as a white solid.

### (2) Synthesis of Compound A1

The following reagents and solvents were placed in a 200-mL recovery flask.

Compound m-3: 1.5 g (3.23 mmol)
Compound m-4: 0.59 g (4.85 mmol)

### Pd(OAc)₂: 0.04 g

### Sphos: 0.13 g

Potassium phosphate: 1.03 g

### Toluene: 75 mL

### H₂O: 5 mL

The reaction solution was heated to reflux with stirring for 6 hours under a stream of nitrogen. After completion of the reaction, water was added thereto, and then liquid-liquid extraction was performed. Dissolution was performed with toluene. Purification was performed by column chromatography (toluene/heptane), and then recrystallization was performed in toluene/heptane to give 5.2 g (yield: 82%) of exemplified compound A1 as a white solid.

Exemplified compound A1 was subjected to mass spectrometry with MALDI-TOF-MS (Bruker Autoflex LRF).

### MALDI-TOF-MS

Measured value: m/z=506
Calculated value: C₄₀H₂₆=506

### Examples 2 to 20 (Synthesis of Exemplified Compounds)

As presented in Tables 2-1 to 2-4, exemplified compounds of Examples 2 to 20 were each synthesized as in Example 1, except that raw materials m-2 and m-4 in Example 1 were changed. The resulting exemplified compounds were subjected to mass spectrometry as in Example 1, and the measured values of m/z are also presented below.

TABLE 2

| Example | Exemplified compound | Raw material m-2 | Raw material m-4 | m/z |
|---|---|---|---|---|
| 2 | 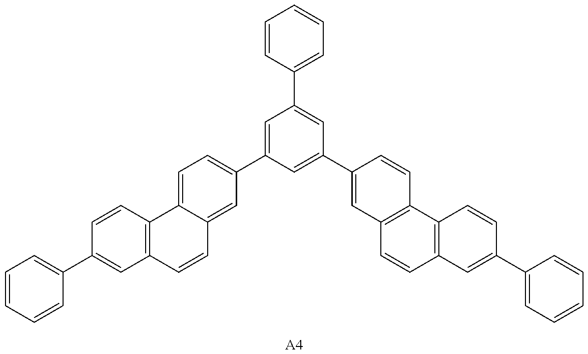 [SMILES: c1ccc(-c2cc(-c3ccc4c(ccc5cc(-c6ccccc6)ccc54)c3)cc(-c3ccc4c(ccc5cc(-c6ccccc6)ccc54)c3)c2)cc1 ; "A4"] | 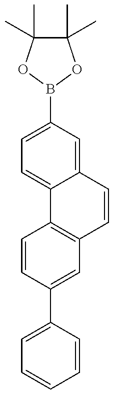 [SMILES: CC1(C)OB(c2ccc3c(ccc4cc(-c5ccccc5)ccc43)c2)OC1(C)C] | 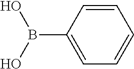 | 658 |
| 3 | 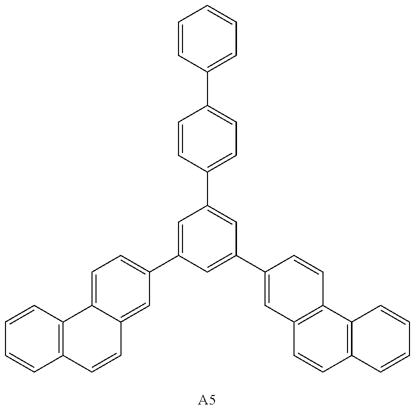 [SMILES: c1ccc(-c2ccc(-c3cc(-c4ccc5c(ccc6ccccc65)c4)cc(-c4ccc5c(ccc6ccccc65)c4)c3)cc2)cc1 ; "A5"] | 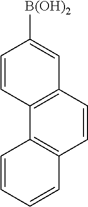 [SMILES: OB(O)c1ccc2c(ccc3ccccc32)c1] | 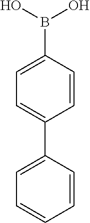 [SMILES: OB(O)c1ccc(-c2ccccc2)cc1] | 582 |
| 4 | 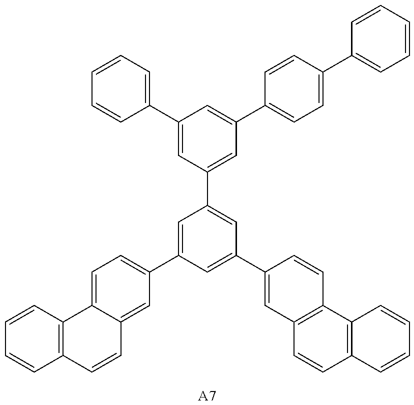 [SMILES: c1ccc(-c2ccc(-c3cc(-c4ccccc4)cc(-c4cc(-c5ccc6c(ccc7ccccc76)c5)cc(-c5ccc6c(ccc7ccccc76)c5)c4)c3)cc2)cc1 ; "A7"] | 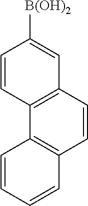 [SMILES: OB(O)c1ccc2c(ccc3ccccc32)c1] | 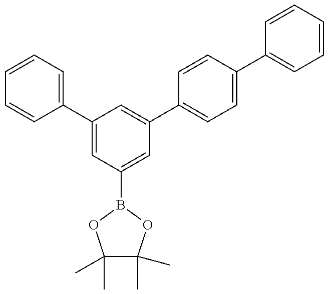 [SMILES: CC1(C)OB(c2cc(-c3ccccc3)cc(-c3ccc(-c4ccccc4)cc3)c2)OC1(C)C] | 734 |
| 5 | 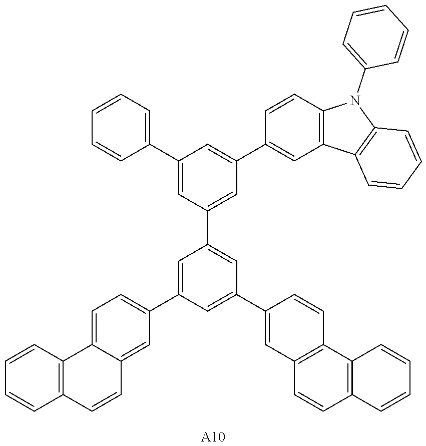 [SMILES: c1ccc(-c2cc(-c3cc(-c4ccc5c(ccc6ccccc65)c4)cc(-c4ccc5c(ccc6ccccc65)c4)c3)cc(-c3ccc4c(c3)c3ccccc3n4-c3ccccc3)c2)cc1 ; "A10"] | 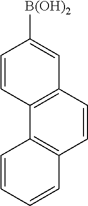 [SMILES: OB(O)c1ccc2c(ccc3ccccc32)c1] | 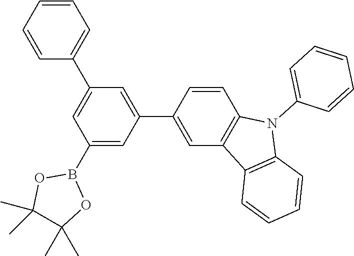 [SMILES: CC1(C)OB(c2cc(-c3ccccc3)cc(-c3ccc4c(c3)c3ccccc3n4-c3ccccc3)c2)OC1(C)C] | 823 |
| 6 | 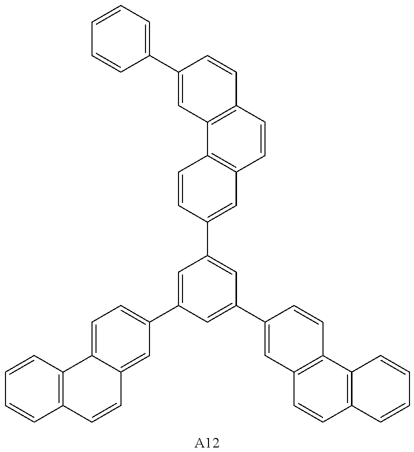 [SMILES: c1ccc(-c2ccc3ccc4cc(-c5cc(-c6ccc7c(ccc8ccccc87)c6)cc(-c6ccc7c(ccc8ccccc87)c6)c5)ccc4c3c2)cc1 ; "A12"] | 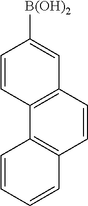 [SMILES: OB(O)c1ccc2c(ccc3ccccc32)c1] | 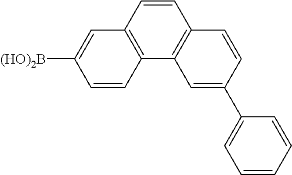 [SMILES: OB(O)c1ccc2c(ccc3ccc(-c4ccccc4)cc32)c1] | 682 |
| 7 | 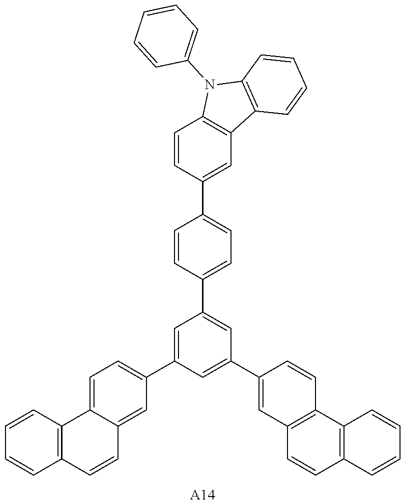 [SMILES: c1ccc(-n2c3ccccc3c3cc(-c4ccc(-c5cc(-c6ccc7c(ccc8ccccc87)c6)cc(-c6ccc7c(ccc8ccccc87)c6)c5)cc4)ccc32)cc1 ; "A14"] | 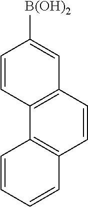 [SMILES: OB(O)c1ccc2c(ccc3ccccc32)c1] | 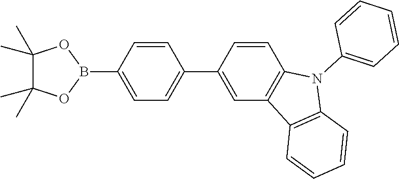 [SMILES: CC1(C)OB(c2ccc(-c3ccc4c(c3)c3ccccc3n4-c3ccccc3)cc2)OC1(C)C] | 747 |
| 8 | 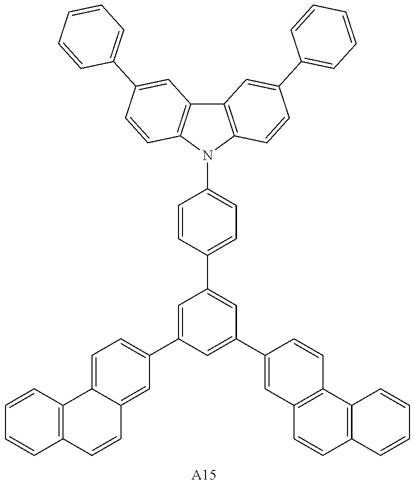 [SMILES: c1ccc(-c2ccc3c(c2)c2cc(-c4ccccc4)ccc2n3-c2ccc(-c3cc(-c4ccc5c(ccc6ccccc65)c4)cc(-c4ccc5c(ccc6ccccc65)c4)c3)cc2)cc1 ; "A15"] | 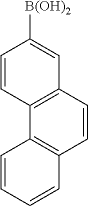 [SMILES: OB(O)c1ccc2c(ccc3ccccc32)c1] | 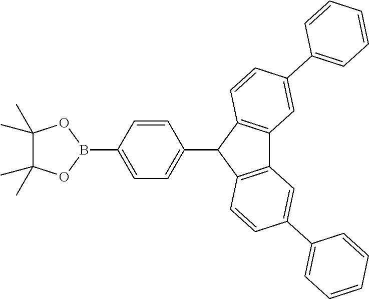 [SMILES: CC1(C)OB(c2ccc(C3c4ccc(-c5ccccc5)cc4-c4cc(-c5ccccc5)ccc43)cc2)OC1(C)C] | 823 |
| 9 | 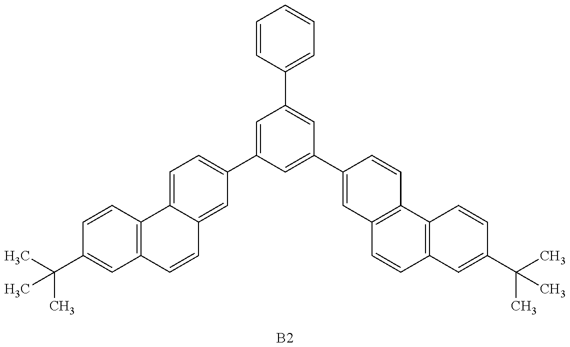 [SMILES: CC(C)(C)c1ccc2c(ccc3cc(-c4cc(-c5ccccc5)cc(-c5ccc6c(ccc7cc(C(C)(C)C)ccc76)c5)c4)ccc32)c1 ; "B2"] | 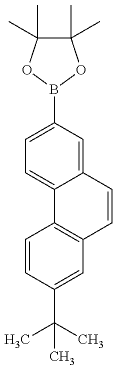 [SMILES: CC(C)(C)c1ccc2c(ccc3cc(B4OC(C)(C)C(C)(C)O4)ccc32)c1] | 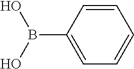 | 618 |
| 10 | 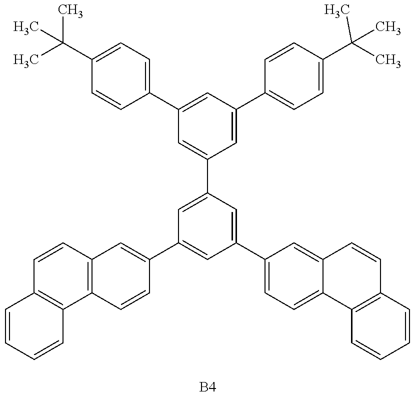 [SMILES: CC(C)(C)c1ccc(-c2cc(-c3ccc(C(C)(C)C)cc3)cc(-c3cc(-c4ccc5c(ccc6ccccc65)c4)cc(-c4ccc5c(ccc6ccccc65)c4)c3)c2)cc1 ; "B4"] | 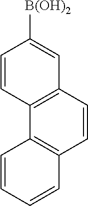 [SMILES: OB(O)c1ccc2c(ccc3ccccc32)c1] | 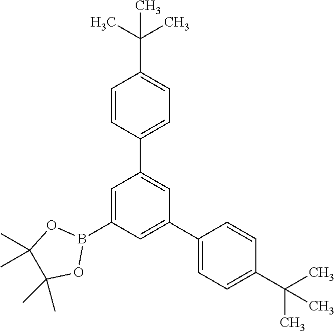 [SMILES: CC(C)(C)c1ccc(-c2cc(B3OC(C)(C)C(C)(C)O3)cc(-c3ccc(C(C)(C)C)cc3)c2)cc1] | 770 |
| 11 | 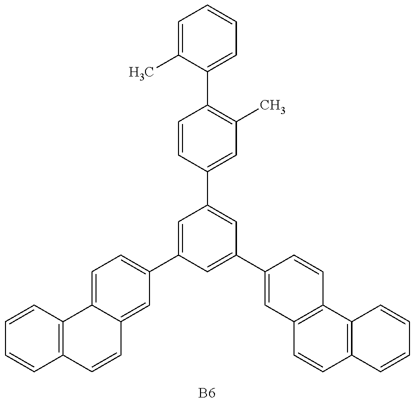 | 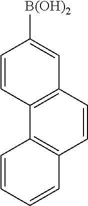 [SMILES: OB(O)c1ccc2c(ccc3ccccc32)c1] | 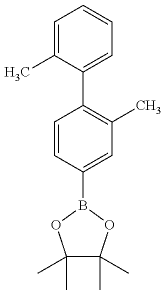 | 610 |
| 12 | 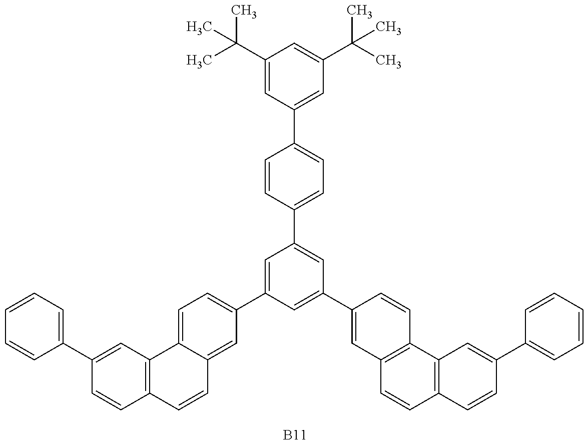 | 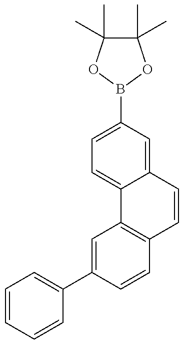 [SMILES: CC1(C)OB(c2ccc3c(ccc4ccc(-c5ccccc5)cc43)c2)OC1(C)C] | 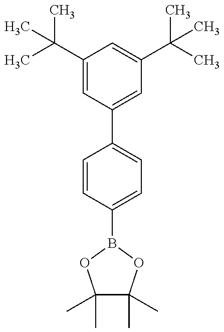 [SMILES: CC(C)(C)c1cc(-c2ccc(B3OC(C)(C)C(C)(C)O3)cc2)cc(C(C)(C)C)c1] | 846 |
| 13 | 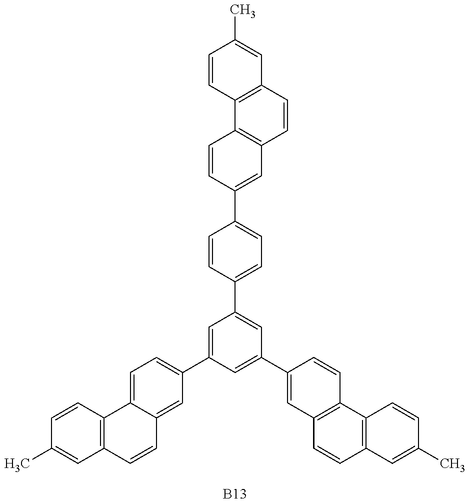 | 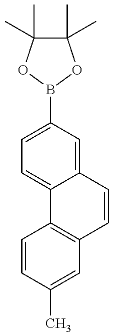 [SMILES: Cc1ccc2c(ccc3cc(B4OC(C)(C)C(C)(C)O4)ccc32)c1] | 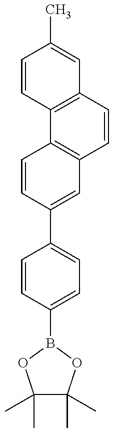 [SMILES: Cc1ccc2c(ccc3cc(-c4ccc(B5OC(C)(C)C(C)(C)O5)cc4)ccc32)c1] | 724 |
| 14 | 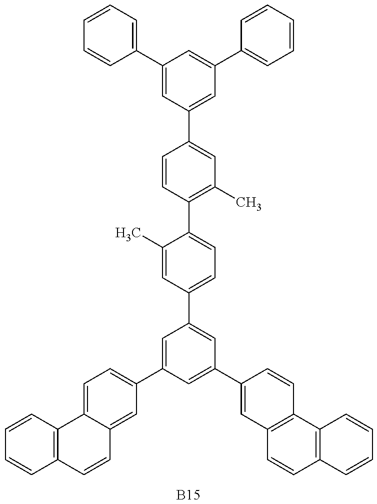 | 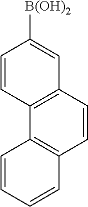 [SMILES: OB(O)c1ccc2c(ccc3ccccc32)c1] | 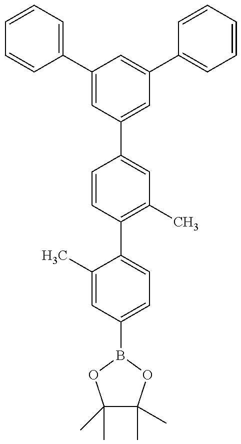 [SMILES: Cc1cc(B2OC(C)(C)C(C)(C)O2)ccc1-c1ccc(-c2cc(-c3ccccc3)cc(-c3ccccc3)c2)cc1C] | 838 |
| 15 | 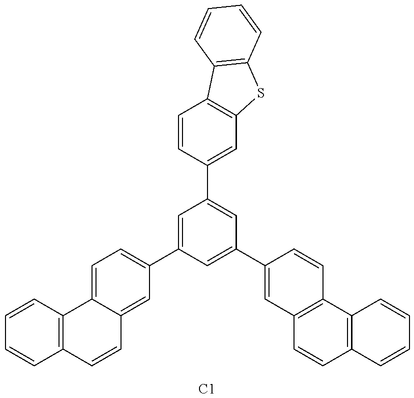 [SMILES: c1ccc2c(c1)ccc1cc(-c3cc(-c4ccc5c(ccc6ccccc65)c4)cc(-c4ccc5c(c4)sc4ccccc45)c3)ccc12 ; "C1"] | 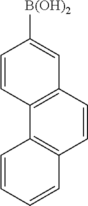 [SMILES: OB(O)c1ccc2c(ccc3ccccc32)c1] | 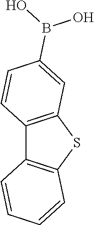 [SMILES: OB(O)c1ccc2c(c1)sc1ccccc12] | 612 |
| 16 | 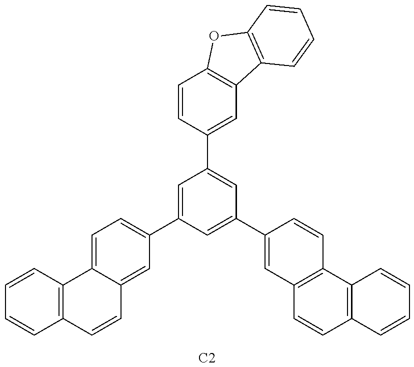 [SMILES: c1ccc2c(c1)ccc1cc(-c3cc(-c4ccc5c(ccc6ccccc65)c4)cc(-c4ccc5oc6ccccc6c5c4)c3)ccc12 ; "C2"] | 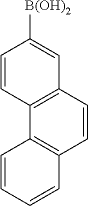 [SMILES: OB(O)c1ccc2c(ccc3ccccc32)c1] | 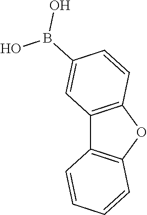 [SMILES: OB(O)c1ccc2oc3ccccc3c2c1] | 596 |
| 17 | 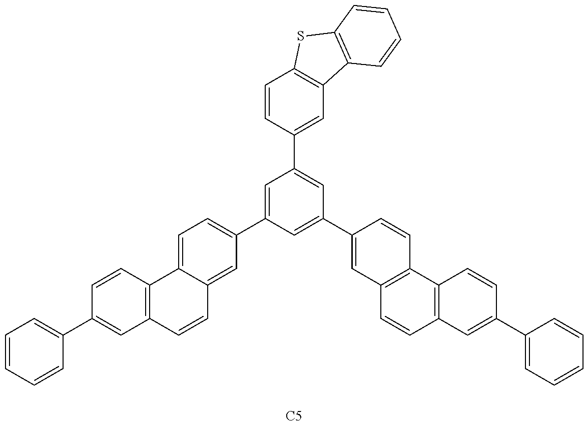 [SMILES: c1ccc(-c2ccc3c(ccc4cc(-c5cc(-c6ccc7c(ccc8cc(-c9ccccc9)ccc87)c6)cc(-c6ccc7sc8ccccc8c7c6)c5)ccc43)c2)cc1 ; "C5"] | 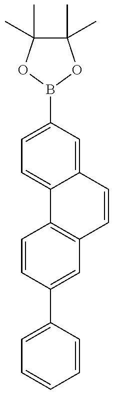 [SMILES: CC1(C)OB(c2ccc3c(ccc4cc(-c5ccccc5)ccc43)c2)OC1(C)C] | 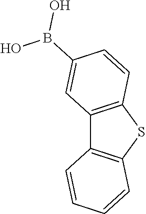 [SMILES: OB(O)c1ccc2sc3ccccc3c2c1] | 764 |
| 18 | 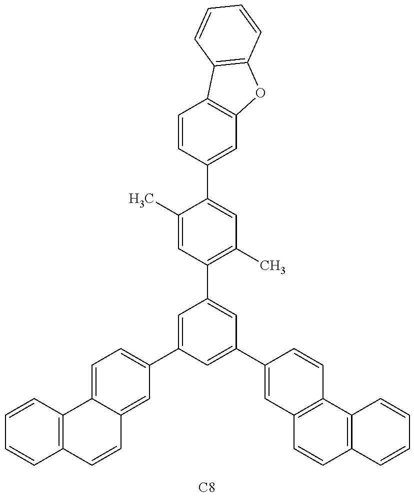 | 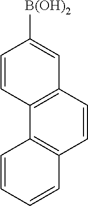 [SMILES: OB(O)c1ccc2c(ccc3ccccc32)c1] | 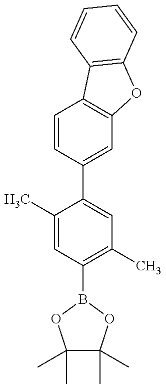 | 700 |
| 19 | 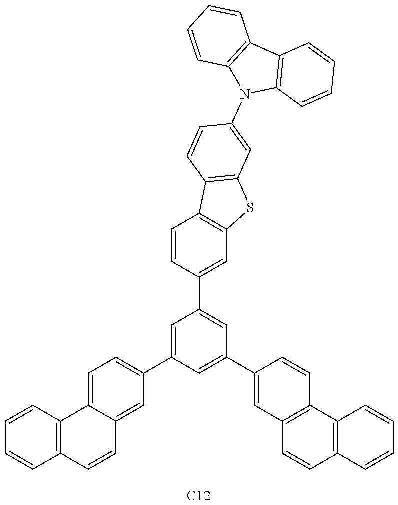 | 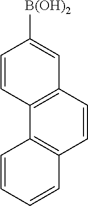 [SMILES: OB(O)c1ccc2c(ccc3ccccc32)c1] | 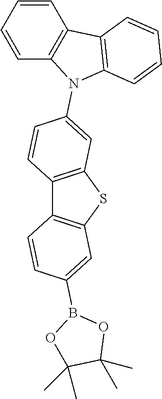 [SMILES: CC1(C)OB(c2ccc3c(c2)sc2cc(-n4c5ccccc5c5ccccc54)ccc23)OC1(C)C] | 777 |
| 20 | 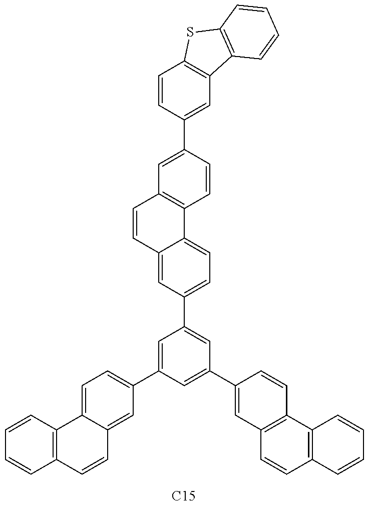 [SMILES: c1ccc2c(c1)ccc1cc(-c3cc(-c4ccc5c(ccc6ccccc65)c4)cc(-c4ccc5c(ccc6cc(-c7ccc8sc9ccccc9c8c7)ccc65)c4)c3)ccc12 ; "C15"] | 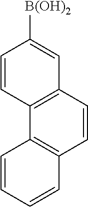 [SMILES: OB(O)c1ccc2c(ccc3ccccc32)c1] | 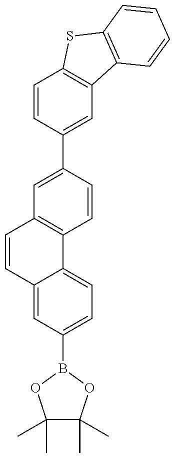 [SMILES: CC1(C)OB(c2ccc3c(ccc4cc(-c5ccc6sc7ccccc7c6c5)ccc43)c2)OC1(C)C] | 788 |

### Example 21

An organic light-emitting device having a bottom-emission structure was produced in which an anode, a hole injection layer, a hole transport layer, an electron-blocking layer, a light-emitting layer, a hole-blocking layer, an electron transport layer, an electron injection layer, and a cathode were sequentially formed on a substrate.

An ITO film was formed on a glass substrate and subjected to desired patterning to form an ITO electrode (anode). The ITO electrode had a thickness of 100 nm. The substrate on which the ITO electrode had been formed in this way was used as an ITO substrate in the following steps. Next, vapor deposition was performed by resistance heating in a vacuum chamber at 1.33×10⁻⁴ Pa to continuously form organic compound layers and an electrode layer presented in Table 3 on the ITO substrate. Here, the opposing electrode (metal electrode layer, cathode) had an electrode area of 3 mm².

TABLE 3

| | | | | Thickness |
|---|---|---|---|---|
| | Material | | | (nm) |
| Cathode | Al | | | 100 |
| Electron injection | LiF | | | 1 |
| layer (EIL) | | | | |
| Electron transport | ET2 | | | 20 |
| layer (ETL) | | | | |
| Hole-blocking | ET11 | | | 20 |
| layer (HBL) | | | | |
| Light-emitting | Host | A4 | Ratio by weight | 20 |
| layer (EML) | Guest | AA13 | A4:AA13 = | |
| | | | 90:10 | |

| | | |
|---|---|---|
| Electron-blocking | HT19 | 15 |
| layer (HBL) | | |
| Hole transport | HT3 | 30 |
| layer (HTL) | | |
| Hole injection | HT16 | 5 |
| layer (HIL) | | |

The characteristics of the resulting device were measured and evaluated. The light-emitting device had a maximum emission wavelength of 524 nm and a maximum external quantum efficiency (E. Q. E.) of 12%.

The device was subjected to a continuous operation test at a current density of 100 mA/cm². The time when the percentage of luminance degradation reached 5% was measured. In the case where the time when the percentage of luminance degradation reached 5% in Comparative example 1 was defined as 1.0, the time (also referred to as a “ratio of luminance degradation”) in this Example was 1.3.

With regard to measurement instruments, in this Example, the current-voltage characteristics were measured with a Hewlett-Packard 4140B microammeter, and the luminance was measured with a Topcon BM7.

### Examples 22 to 39 and Comparative Examples 1 to 3

Organic light-emitting devices in Example 22 to 39 were produced in the same manner as in Example 21, except that compounds listed in Table 4 were used as appropriate. The characteristics of the resulting devices were measured and evaluated as in Example 21. Table 4 presents the measurement results.

TABLE 4

| | | | | | | | | | Ratio of |
|---|---|---|---|---|---|---|---|---|---|
| | | | | EML | | | | E. Q. E. | luminance |
| | HIL | HTL | EBL | Host | Guest | HBL | ETL | [%] | degradation |
| Example 22 | HT16 | HT3 | HT19 | A1 | AA2 | ET12 | ET15 | 11 | 1.2 |
| Example 23 | HT16 | HT2 | HT19 | A4 | AA26 | ET12 | ET2 | 13 | 1.3 |
| Example 24 | HT16 | HT2 | HT19 | A5 | AA27 | ET11 | ET2 | 12 | 1.3 |
| Example 25 | HT16 | HT3 | HT19 | A7 | CC16 | ET11 | ET2 | 11 | 1.2 |
| Example 26 | HT16 | HT3 | HT19 | A10 | GD10 | ET11 | ET2 | 12 | 1.3 |
| Example 27 | HT16 | HT3 | HT19 | A14 | HH3 | ET11 | ET15 | 13 | 1.4 |
| Example 28 | HT16 | HT3 | HT19 | A15 | BB25 | ET12 | ET2 | 14 | 1.3 |
| Example 29 | HT16 | HT2 | HT15 | B2 | BB25 | ET12 | ET15 | 12 | 1.1 |
| Example 30 | HT16 | HT3 | HT19 | B4 | GD10 | ET12 | ET15 | 12 | 1.1 |
| Example 31 | HT16 | HT2 | HT15 | B6 | DD6 | ET11 | ET2 | 11 | 1.2 |
| Example 32 | HT16 | HT3 | HT19 | B12 | DD32 | ET12 | ET15 | 12 | 1.3 |
| Example 33 | HT16 | HT2 | HT15 | B13 | DD28 | ET12 | ET2 | 13 | 1.2 |
| Example 34 | HT16 | HT2 | HT15 | B15 | EE1 | ET11 | ET2 | 13 | 1.3 |
| Example 35 | HT16 | HT3 | HT15 | C2 | EE3 | ET12 | ET15 | 11 | 1.3 |
| Example 36 | HT16 | HT3 | HT19 | C5 | FF34 | ET12 | ET15 | 12 | 1.3 |
| Example 37 | HT16 | HT3 | HT19 | C7 | HH19 | ET11 | ET15 | 12 | 1.2 |
| Example 38 | HT16 | HT3 | HT19 | C8 | FF1 | ET12 | ET15 | 12 | 1.1 |
| Example 39 | HT16 | HT3 | HT19 | C12 | GG14 | ET11 | ET15 | 12 | 1.2 |
| Comparative | HT16 | HT3 | HT19 | Comparative | GD10 | ET11 | ET2 | 8 | 1.0 |
| example 1 | | | | compound 1-A | | | | | |
| Comparative | HT16 | HT3 | HT19 | Comparative | GD10 | ET11 | ET2 | 9 | 1.0 |
| example 2 | | | | compound 1-B | | | | | |
| Comparative | HT16 | HT3 | HT19 | Comparative | GD10 | ET11 | ET2 | 8 | 0.8 |
| example 3 | | | | compound 1-C | | | | | |

As can be seen from Table 4, the maximum external quantum efficiencies (E. Q. E.) of Comparative examples 1 to 3 were 8%, 9%, and 8%, respectively, and the light-emitting devices according to embodiments of the present disclosure were superior in luminous efficiency. This is because the exemplified compounds according to embodiments of the present disclosure had small ΔST values.

In addition, the light-emitting devices according to embodiments of the present disclosure were superior in device lifetime.

In particular, by using the organic compounds according to embodiments of the present disclosure and, as the guest materials, the materials having ligands each containing a phenanthrenyl group, a benzofluorenyl group, a dibenzofuranyl group, a triphenylene group, a naphthoisoquinoline group, or a benzoisoquinoline group and being suitable for combinations with the organic compounds according to the embodiments of the present disclosure, the light-emitting devices had improved device lifetimes and luminous efficiencies.

As described above, it is possible to provide a light-emitting device excellent in luminous efficiency and device lifetime by using the organic compound according to an embodiment of the present disclosure.

### Example 40

An organic light-emitting device was produced in the same manner as in Example 21, except that organic compound layers and an electrode layer given in Table 5 were continuously formed.

TABLE 5

| | | | | Thickness |
|---|---|---|---|---|
| | Material | | | (nm) |
| Cathode | Al | | | 100 |
| Electron injection | LiF | | | 1 |
| layer (EIL) | | | | |
| Electron transport | ET2 | | | 20 |
| layer (ETL) | | | | |
| Hole-blocking | ET11 | | | 20 |
| layer (HBL) | | | | |
| Light-emitting | Host | A7 | Ratio by weight | 20 |
| layer (EML) | Guest | AA21 | A7:AA21:EM29 = | |
| | Assist | EM29 | 60:10:30 | |

| | | |
|---|---|---|
| Electron-blocking | HT19 | 15 |
| layer (HBL) | | |
| Hole transport | HT3 | 30 |
| layer (HTL) | | |
| Hole injection | HT16 | 5 |
| layer (HIL) | | |

The characteristics of the resulting device were measured and evaluated. The emission color of the light-emitting device was green, and the maximum external quantum efficiency (E. Q. E.) was 17%.

### Examples 41 to 60

Organic light-emitting devices in Example 41 to 60 were produced in the same manner as in Example 40, except that compounds listed in Table 6 were used as appropriate. The characteristics of the resulting devices were measured and evaluated as in Example 40. Table 6 presents the measurement results.

TABLE 6

| | | | | EML | | | | | E. Q. E. |
|---|---|---|---|---|---|---|---|---|---|
| | HIL | HTL | EBL | Host | Guest | Assist | HBL | ETL | [%] |
| Example 41 | HT16 | HT3 | HT19 | A1 | BB21 | EM30 | ET26 | ET3 | 16 |
| Example 42 | HT16 | HT2 | HT19 | A4 | BB24 | EM31 | ET13 | ET2 | 17 |
| Example 43 | HT16 | HT2 | HT19 | A5 | CC16 | EM29 | ET13 | ET2 | 16 |
| Example 44 | HT16 | HT3 | HT15 | A7 | DD31 | EM28 | ET16 | ET15 | 17 |
| Example 45 | HT16 | HT3 | HT19 | A10 | DD26 | EM30 | ET17 | ET15 | 18 |
| Example 46 | HT16 | HT3 | HT19 | A14 | EE3 | EM39 | ET13 | ET2 | 17 |
| Example 47 | HT16 | HT2 | HT15 | A15 | EE2 | GD10 | ET15 | ET3 | 18 |
| Example 48 | HT16 | HT3 | HT19 | B2 | FF2 | ET15 | ET15 | ET15 | 16 |
| Example 49 | HT16 | HT2 | HT15 | B4 | FF22 | ET16 | ET2 | ET2 | 16 |
| Example 50 | HT16 | HT3 | HT19 | B6 | FF2 | EM16 | ET26 | ET3 | 16 |
| Example 51 | HT16 | HT2 | HT15 | B12 | GG2 | EM16 | ET13 | ET2 | 15 |
| Example 52 | HT16 | HT2 | HT15 | B13 | HH26 | EM34 | ET13 | ET2 | 15 |
| Example 53 | HT16 | HT3 | HT19 | C1 | DD32 | EM31 | ET15 | ET15 | 16 |
| Example 54 | HT16 | HT3 | HT19 | C2 | GG22 | EM34 | ET16 | ET15 | 16 |
| Example 55 | HT16 | HT3 | HT19 | C7 | DD47 | EM35 | ET16 | ET15 | 17 |
| Example 56 | HT16 | HT2 | HT15 | C8 | DD36 | EM30 | ET15 | ET3 | 15 |
| Example 57 | HT16 | HT2 | HT15 | C12 | DD8 | EM28 | ET2 | ET2 | 16 |
| Example 58 | HT16 | HT2 | HT15 | C13 | HH26 | EM30 | ET13 | ET2 | 18 |
| Example 59 | HT16 | HT3 | HT19 | B13 | II2 | GD12 | ET13 | ET3 | 16 |
| Example 60 | HT16 | HT3 | HT19 | B15 | II3 | GD10 | ET26 | ET2 | 16 |

As can be seen from Table 6, by using the organic compounds according to embodiments of the present disclosure and, as the assist materials, the materials each containing a pyridyl group, a carbazolyl group, a triazinyl group, a xanthone group, or a thioxanthone group and being suitable for combinations with the organic compounds according to the embodiments of the present disclosure, the light-emitting devices had improved luminous efficiencies.

As described above, the organic compound according to an embodiment of the present disclosure has small ΔST. Thus, when the organic compound according to an embodiment of the present disclosure is used for an organic light-emitting device, it is possible to provide the organic light-emitting device having a low device driving voltage and an excellent device lifetime.

According to an embodiment of the present disclosure, when the organic compound according to an embodiment of the present disclosure is used in an organic light-emitting device, it is possible to provide the organic light-emitting device excellent in device lifetime.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-083821, filed May 23, 2022, which is hereby incorporated by reference herein in its entirety.

What is claimed is:

1. An organic compound represented by the following formula (1): 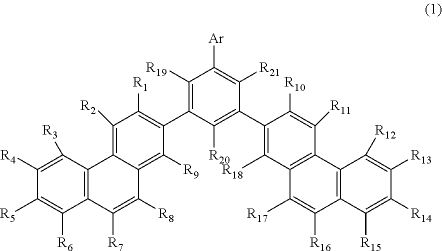
where in formula (1), Ar is a substituted or unsubstituted carbazolyl group, a substituted or unsubstituted aryl group, or a substituted or unsubstituted heterocyclic group containing a chalcogen element, provided that when Ar is a phenanthrenyl group, the phenanthrenyl group has a substituent, R₁ to R₂₁ are each independently selected from the group consisting of a hydrogen atom, a deuterium atom, a halogen atom, a substituted or unsubstituted alkyl group, a substituted or unsubstituted alkoxy group, a substituted or unsubstituted silyl group, a substituted or unsubstituted amino group, a substituted or unsubstituted aryl group, a substituted or unsubstituted heterocyclic group, and a substituted or unsubstituted carbazolyl group.

2. The organic compound according to claim 1, wherein in formula (1), Ar is represented by the following formula (2): 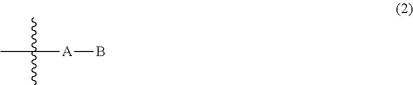
where in formula (2), A is a substituted or unsubstituted phenylene group, a substituted or unsubstituted biphenylene group, or a direct bond.

3. The organic compound according to claim 2, wherein formula (2) is represented by any of the following formulae (3) to (9): 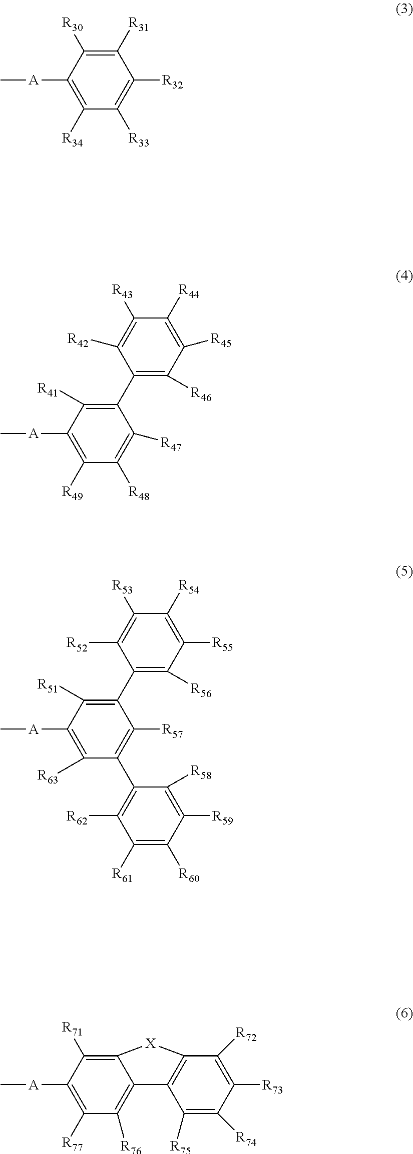 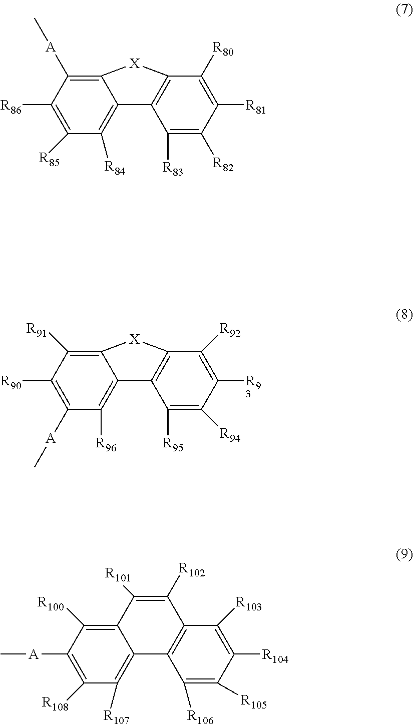
where in formulae (3) to (9), each X is a chalcogen element, R₃₀ to R₁₀₈ are each independently selected from the group consisting of a hydrogen atom, a deuterium atom, a halogen atom, a substituted or unsubstituted alkyl group, a substituted or unsubstituted alkoxy group, a substituted or unsubstituted silyl group, a substituted or unsubstituted amino group, a substituted or unsubstituted aryl group, and a substituted or unsubstituted heterocyclic group, and in formula (9), when A is a direct bond, at least one of R₁₀₀ to R₁₀₈ contains a substituent.

4. The organic compound according to claim 1, wherein in formula (1), Ar further contains a deuterium atom, a carbazole skeleton, an alkyl group having 1 to 4 carbon atoms, an aryl group having 6 to 18 carbon atoms, a chalcogen element-containing heterocyclic group having 8 to 12 carbon atoms, a silyl group having an aryl group, or an amino group containing an alkyl group or an aryl group.

5. The organic compound according to claim 1, wherein in formula (1), at least one selected from the group consisting of R₄, R₅, R₆, R₇, R₁₃, R₁₄, and R₁₆ contains a substituent.

6. The organic compound according to claim 1, wherein in formula (1), at least one of R₁ to R₁₉ is a deuterium atom, an alkyl group having 1 to 4 carbon atoms, or an aryl group having 6 to 12 carbon atoms.

7. The organic compound according to claim 1, wherein the organic compound contains no SP3 carbon.

8. The organic compound according to claim 1, wherein in formula (1), Ar contains no electron-withdrawing substituent.

9. An organic light-emitting device, comprising:
a first electrode;
a second electrode; and
an organic compound layer disposed between the first electrode and the second electrode,
wherein the organic compound layer contains the organic compound according to claim 1.

10. The organic light-emitting device according to claim 9, wherein the organic compound layer contains a light-emitting layer, and
the light-emitting layer contains the organic compound.

11. The organic light-emitting device according to claim 10, wherein the light-emitting layer further contains a first compound, and
the first compound has lower lowest excited triplet energy than the organic compound.

12. The organic light-emitting device according to claim 11, wherein the first compound is a phosphorescent material, and
the organic compound is contained in a proportion of 30% to 99% by weight based on an entire light-emitting layer.

13. The organic light-emitting device according to claim 11, wherein the first compound contains a fused ring containing at least three rings.

14. The organic light-emitting device according to claim 11, wherein the first compound contains an aryl group having 12 to 20 carbon atoms or a heterocyclic group 12 to 18 carbon atoms.

15. The organic light-emitting device according to claim 11, wherein the first compound contains a phenanthrene skeleton, a fluorene skeleton, a benzofluorene skeleton, a dibenzofuran skeleton, a triphenylene skeleton, a dibenzothiophene skeleton, a naphthoisoquinoline skeleton, or a benzoisoquinoline skeleton.

16. The organic light-emitting device according to claim 11, wherein the light-emitting layer further contains a second compound,
lowest excited triplet energy of the second compound is equal to or higher than the lowest excited triplet energy of the first compound.

17. The organic light-emitting device according to claim 16, wherein the second compound contains a pyridine skeleton, an azine ring, a carbazole skeleton, a xanthone skeleton, or a thioxanthone skeleton.

18. A display apparatus, comprising:
multiple pixels,
at least one of the multiple pixels including:
the organic light-emitting device according to claim 9, and
a transistor coupled to the organic light-emitting device.

19. A photoelectric conversion apparatus, comprising:
an optical unit including multiple lenses;
an image pickup device configured to receive light passing through the optical unit; and
a display unit configured to display an image captured by the image pickup device,
wherein the display unit includes the organic light-emitting device according to claim 9.

20. An electronic apparatus, comprising:
a display unit including the organic light-emitting device according to claim 9;
a housing provided with the display unit; and
a communication unit being disposed in the housing and communicating with an outside.

21. A lighting apparatus, comprising:
a light source including the organic light-emitting device according to claim 9; and
a light diffusion unit or an optical film configured to transmit light emitted from the light source.

22. A moving object, comprising:
a lighting unit including the organic light-emitting device according to claim 9; and
a body provided with the lighting unit.

23. An image-forming apparatus, comprising:
a photoconductor; and
an exposure light source configured to expose the photoconductor,
wherein the exposure light source includes the organic light-emitting device according to claim 9.
